(19) United States
(12) Patent Application Publication
(10) Pub. No.: US 2026/0259205 A1
(43) Pub. Date: Sep. 3, 2026

(54) ELECTRONIC BIOSENSORS COMPRISING METAL-NONMETAL COMPOUND SEMICONDUCTOR NANOBRIDGE ARRAY

(71) Applicant: ROSWELL ME, INC., San Diego, CA (US)

(72) Inventors: Sungho Jin, San Deigo, CA (US); Barry Merriman, San Deigo, CA (US); Prem Kumar Sinha, San Deigo, CA (US); Carl Fuller, Berkeley Heights, NJ (US); Chulmin Choi, San Deigo, CA (US); Paul W. Mola, San Deigo, CA (US)

(73) Assignee: SemiconBio,Inc., San Diego, CA (US)

(21) Appl. No.: 19/165,494

(22) PCT Filed: Apr. 15, 2024
(86) PCT No.: PCT/US2024/024562

§ 371 Date: Sep. 15, 2025

Related U.S. Application Data
(60) Provisional application No. 63/459,246, filed on Apr. 13, 2023.

(51) Int. Cl. G01N 33/543; G01N 27/414

(52) U.S. Cl. CPC G01N 33/5438; G01N 27/4145

(57) ABSTRACT

The disclosure relates to an array of biosensor, at least one biosensor in the array having: an electrode pair having a source electrode and a drain electrode separated by a nanogap; a nanobridge including a metal-nonmetal compound semiconductor connecting the two electrodes across the nanogap; one or more bioreceptors attached to the nanobridge; an analyte solution comprising water, salts and one or more target molecules; a reaction chamber for housing the array and for receiving the analyte solution; a circuitry configured to detect and measure a change in electrical characteristics of the analyte when a respective one of the target molecules attaches or detaches from a respective one of the bioreceptors; wherein the biosensor detects a change in the electrical characteristic of the nanobridge during the event.

### PRIORITY

The disclosure claims priority to the PCT Application Serial No. PCT/US24/24562 (Published as WO2024220344) filed Apr. 15, 2024, which itself claims priority to the U.S. Provisional Application No. 63/459,246, filed Apr. 13, 2023; the disclosure of the foregoing applications are incorporated herein in their entirety.

The disclosure relates to the following patents and patent applications U.S. Pat. No. 10,712,334 (issued on Jul. 14, 2020), U.S. Pat. No. 10,737,263 (issued on Aug. 11, 2020), U.S. Pat. No. 11,656,197 (issued May 23, 2023) and Publication No. US 2020/0393440A1 (filed May 6, 2020), and Patent Publication No. US 2019/0376925A1 (filed Nov. 22, 2017). The disclosure of the forgoing patents and publications are incorporated herein in their entirety for background information.

### FIELD

The disclosure relates to the metal-nonmetal compound semiconductors including oxide base semiconductor bridges comprising biological sensor molecules for analyte detection, measurement of molecular interaction kinetics, DNA or genome sequencing applications, detecting disease-inducing microorganisms or pathogens, and digital data storage in molecular structures.

### BACKGROUND

In recent years, significant progress has been made in biosensor technologies. Biosensors are commonly utilized for detection, quantification and characterization of analytes that are biomolecules. Such sensors have applications as diagnostics, including point-of-care diagnostics, for the detection of pathogens, such as disease-inducing or epidemic-causing bacteria, viruses or other microorganisms, including the COVID-19 virus, for monitoring metabolites or electrolytes in biofluids, for the analysis of DNA sequence and genome sequencing, for forensic analysis of proteins or DNAs, sensing of environmental pathogens, allergens, or pollutants (organic or inorganic), monitoring drug-target molecular interactions for drug development (organic or inorganic), and information storage (e.g., using molecules such as DNAs, RNAs, proteins, DNA origami structures, antibodies, aptamers, or enzymes to store or retrieve digital information). Different techniques may be employed for the detection of various analytes in biosensors, for example, producing change in measurable physical properties such as electrical signals, optical responses, magnetic, chemical, thermal or biological changes in response to, and qualitatively proportional to, the quantity of specific analytes attaching to and collected by the biomolecular biosensor.

There are a number of desirable features for biosensor devices. Specificity of the biosensors, i.e., ability to respond only to the intended analytes is an important characteristic of a desirable biosensor. Sensitivity of the biosensor, i.e., ability to respond to small amounts of the intended analytes is another important characteristic of desirable biosensors. Other important basic properties are rapid or real-time response time when exposed to the analyte, label-free detection of analytes, universality, i.e., the ability to be directed or programmed for many different analyte targets, and scalability, i.e., the ability to deploy many independent biosensors on one device.

Many biosensors comprise a “probe” or “receptor” molecule that interacts with the target analyte, as part of the signal transduction process. These bioreceptor or biosensor probe molecules are conveniently attached to an elongated ribbon-like or wire-like structure (a “bridge”), that is long relative to its thickness and width, made of semiconductor material, to form a field effect transistor (“FET”) sensor device or related semiconductor sensor device, which also comprises a positive electrode (source electrode) and a negative electrode (drain electrode), optionally aided by one or more auxiliary electrode structures that are insulated from the primary electrodes and used to apply modulating or control voltages (gate electrodes). Such devices function as sensors by having a measurable electrical property, such as conductivity or device current, which is altered when the probe molecules are engaged with the target analyte. Such sensors may in preferred embodiments comprise a single probe molecule, or multiple probe molecules.

### SUMMARY

In one embodiment, the disclosure relates to an array of biosensor, at least one biosensor in the array having: an electrode pair having a source electrode and a drain electrode separated by a nanogap; a nanobridge including a metal-nonmetal compound semiconductor connecting the two electrodes across the nanogap; one or more bioreceptors attached to the nanobridge; an analyte solution comprising water, salts and one or more target molecules; a reaction chamber for housing the array and for receiving the analyte solution; a circuitry configured to detect and measure a change in electrical characteristics of the analyte when a respective one of the target molecules attaches or detaches from a respective one of the bioreceptors; wherein the biosensor detects a change in the electrical characteristic of the nanobridge during the event. In one application, the plurality of bioreceptors wherein the placement of the bioreceptors on the nanobridge is predefined by a mask layer having a plurality of openings to couple each bioreceptor to the nanobridge through one of the respective openings.

In one embodiment, the signal-to-noise ratio (SNR) in such bioreceptor-based molecular sensors is increased by making the dimension of the sensor-molecule-containing bridge smaller, for example, by (i) using a pre-made, small diameter biopolymer bridges such as DNA, RNA, or peptides, or (ii) utilizing a small dimension (thin and narrow) inorganic semiconductor bridge such as oxide semiconductors, graphene, MoS₂ and other nanowire-shaped or nano-ribbon shaped materials. When the sensor bridge is made even thinner and/or narrower, the baseline electrical resistance (or impedance) becomes higher, and hence the small electrical signal (or the change in electrical conductivity, measured by a change in electrical current, voltage or capacitance) induced by an analyte attachment event onto the bioreceptor will have a relatively larger impact on the overall signal magnitude.

In certain disclosed embodiments, novel biosensors with label-free, improved electrical sensing capabilities via structural and dimensional control are disclosed, in particular, with nanoscale bridges selected from metal oxide semiconductors, such as indium oxide (In₂O₃), or indium tin oxide, indium zinc oxide, indium gallium zinc oxide, or other oxides, metal nitride semiconductors, metal phosphide semiconductors, metal sulfide semiconductors, or metal carbide semiconductors. These semiconductors are desirable as they are stable due to the fact that they were already oxidized or nitrided, or already in the form of stable semiconductor carbide, phosphide or sulfide.

In terms of the biosensor structural configurations, one or more sensor probe biomolecules such as DNA polymerase enzyme molecules or DNA oligos, or DNA or RNA aptamers, or antibodies, or antigens are precisely positioned on the nanobridge so that a controlled and repeatable sense signals may be readily obtained. For some preferred embodiments, a single probe molecule is provided, preferably in a controlled or localized location on the nanobridge, and for other preferred embodiments, two or multiple sensor probe molecules may be provided, preferably repeatably located or organized in a defined spatial pattern on each nanobridge could provide enhanced sensor reliability/availability and more rapid biosensing operations.

In another embodiment, the disclosure relates to a method for preparing an array of biosensor structures for biosensing operations, each biosensor comprising: a source-drain nanoelectrode pair made of a conductive metal, with the electrodes separated by a nanogap, wherein the conductive metal material is selected from the group consisting of Au, Pt, Pd, Ag, Ru, Rh, Ir, Cu, Ni and alloys thereof; a nanobridge disposed in the nanogap and connecting the source and the drain electrodes, the nanobridge comprising a metal-nonmetal compound semiconductor connecting each of the two electrode pairs across the nanogap, wherein the metal-nonmetal compound semiconductor selected from an oxide, carbide, nitride, phosphide or sulfide semiconductor; a bioreceptor biomolecules coupled to the semiconductor nanobridge to attract and bindingly engage with a target molecule, with the number and location of the bioreceptor molecules controlled by a mask layer comprising pre-defined openings to allow bioreceptor tethering attachment; wherein the bioreceptor binds to the nanobridge by one or more inserted conjugating moiety molecules selected from the group consisting of biotin-streptavidin complex, antigen-antibody complex, pyrene-maleimide complex, connecting moiety comprising azide-related -related click chemistry, amine-related click chemistry, tag-binding related links, and silane-related complexes.

In addition, the disclosure provides novel biosensors with label-free, improved electrical sensing capabilities via structural and dimensional control. The disclosed invention devices may utilize various bioreceptor sensor probe molecules such as one or more enzymes molecules (e.g., DNA polymerase, CRISPR Cas), antibodies, aptamers, DNA oligos, antigens, or cells or viral particles.

In some embodiments, the disclosure provides various methods of fabricating such novel biosensors, including in-situ nanofabrication on semiconductor wafers or chip device surfaces, or alternatively, methods of transfer depositing on the device substrate one or more (or an array) of dimension-optimized or semiconductor-characteristics-optimized nanobridges already prepared on another substrate, and methods of constructing biomolecular sensors comprising nanobridges or microbridges, polymerase type sensor molecules, other sensor probe molecules, and other gate electrodes, support structures, electrical contact structures, or passivation or protective structures.

The disclosed devices are useful for various biomedical or non-biomedical applications such as diagnostic testing, point-of-care diagnostics, detection of analytes (including but not limited to) disease-inducing or epidemic-causing bacteria, virus including COVID-19 virus, or other microorganisms, nucleotides in DNA sequence analysis and genome sequencing, protein or DNA analysis in forensic technology, metabolic and electrolytic profiling in sweat or other body fluid analysis, sensing of environmental pathogens, allergens, or pollutants (organic or inorganic), drug development (organic or inorganic), and information storage (e.g., storing information in DNAs, RNAs, proteins, DNA origami structures, aptamers, or enzyme molecules).

### BRIEF DESCRIPTION OF THE DRAWINGS

The features and advantages of the embodiments of the present disclosure will become more apparent from the detailed description set forth below when taken in conjunction with the drawings. The drawings and the associated descriptions are provided to illustrate embodiments of the disclosure. The drawing and illustrations do not limit the scope of what is claimed.

FIG. 1(a) to 1(c) schematically illustrates a metal oxide, metal carbide, or metal nitride based ultrathin semiconductor nanobridge array for biomolecular sensors. FIG. 1(a) illustrates a nanobridge patterned or stamp transferred on top of source-drain metal electrode pair, wherein inherent van der Waals force of nanolayered materials are utilized for anchoring. FIG. 1(b) illustrates a nanobridge secured under metal electrodes, with the metal electrodes deposited after the nanobridge is first placed. FIG. 1(c) illustrates a nanobridge on top of metal electrodes, but with enhanced nanobridge adhesion by an added anchoring structure on the transfer deposited nanobridge surface.

FIG. 2(a) to 2(d) illustrate various methods that may be utilized to prepare an oxide semiconductor thin film such as an In₂O₃ film for fabrication of nanobridge biosensors according to certain disclosed embodiments. FIG. 2(a) illustrates physical vapor deposition (PVD) on a substrate; FIG. 2(b) illustrates chemical vapor deposition on a substrate; FIG. 2(c) shows spin coating a precursor salt solution, which is subsequently thermally processed and converted into the final compound semiconductor form, such as an In₂O₃ film; and FIG. 2(d) shows metal film deposited on a substrate, subsequently oxidized into a metal oxide film, such as an In₂O₃ film.

FIG. 3(a) to 3(d) illustrate thickness reduction and width reduction of ultrathin semiconductor nanobridge using a chemical etch or plasma etch for erosion from the side edges and from the top of the nanoribbon according to certain embodiments. Specifically, FIG. 3(a) illustrates the placement of the nanobridge. FIG. 3(b) shows the addition of etch barrier; FIG. 3(c) illustrates the etching of the nanobridge; and FIG. 3(d) illustrates a cross section of the etched nanobridge.

FIG. 4 illustrates using erodible mask line array for creation of width-reduced and aligned semiconductor nanoribbon array on a flat substrate, either directly on the sensor electrode pair or for subsequent transfer deposition onto device electrode pair array to form biosensor nanobridges. Specifically, FIG. 4(a) illustrates semiconductor film deposition on a substrate; FIG. 4(b) illustrates deposition and patterning of an etch mask layer; FIG. 4(c) illustrates etching away the semiconductor layer; FIG. 4(d) illustrates the process of gradual erosion of the mask line and resultant narrower semiconductor line using the width-reduced mask line.

FIG. 5 is an example of transfer deposition of pre-made metal oxide type nanoribbons on a separate substrate, for electrode nanobridge formation according to one embodiment of the disclosure. Specifically, FIG. 5(a) illustrates an exemplary process of stamp transfer onto biosensor device (source-drain electrode array not shown); FIG. 5(b) illustrates a nanobridge array placed on top of the device source-drain metallic electrode pair; FIG. 5(c) and 5(d) illustrate nanobridge array positioned underneath or above the metal electrodes.

FIG. 6 illustrates an embodiment for adhesion stability and control of transfer-deposited metal oxide, metal nitride or metal carbide nanoribbon nanobridges on source and drain electrode surface. Specifically, FIG. 6(a) shows stamp transfer using the van der Waals boding force according to one embodiment of the disclosure; FIG. 6(b) shows an anchoring structure to ensure physical stability of the nanobridge according to one embodiment of the disclosure.

FIG. 7(a) and FIG. 7(b) illustrates two alternative sequences for the placement of the semiconducting oxide film relative to the metal electrode array placement according to one embodiment of the disclosure.

FIG. 8 illustrates a top view of an array of metal electrode pairs with stamp-transferred inorganic nanoribbon bridges such as oxide, nitride or carbide type, with redundant ribbons to ensure at least one bridge formation occurs. Specifically, FIG. 8(a) shows an array of electrode conducting wires for signal detection; FIG. 8(b) shows the optional deposition of anchoring metal(s); and FIG. 8(c) shows a unit of FET sensor with an enzyme polymerase according to one embodiment of the disclosure.

FIG. 9 illustrates amorphous and crystalline oxide semiconductors for biomolecular sensors according to the disclosed embodiments.

FIG. 10 illustrates an insulating gate arrangement according to some embodiment for the disclosure. Specifically, FIG. 10(a) shows two electrode, no-added-gate FET molecular biosensor; FIG. 10(b) illustrates a semiconductor nanobridge such as In₂O₃ connecting the source-drain electrode with a planarizing filler between the source and the drain, FIG. 10(c) shows insulator-gated FET molecular biosensor with a thin insulating layer (SiO₂, Si₃N₄, Al₂O₃, refractory metal oxide, rare-earth oxide, etc) serving a dual purpose of anti-corrosion and insulator-gating above which the single enzyme molecule with DNA template (not shown here) serves as a modulating gate on nucleotide attachment.

FIG. 11 illustrates an insulator-gated FET molecular biosensor, with a very thin insulating layer above which the single enzyme molecule with a DNA template is present which serves as a modulating gate on nucleotide attachment.

FIG. 12 is a top view of various conductor gated structures of oxide-semiconductor-nanobridge based molecular biosensors. The top gate structure comprises a separate gate positioned above the polymerase molecular sensor molecule attached on top of the In₂O₃ type semiconductor nanobridge. The gate (e.g., Si or other metallic back gate) may also be positioned on the bottom region of the electrode pair, side in a parallel orientation or perpendicular orientation, or on the side of the electrode pair in a parallel orientation or perpendicular orientation.

FIG. 13 shows various methods of semiconductor thin film releasing/detaching for biosensor applications (e.g., In₂O₃ film of e.g., 0.5-20 nm thick) from a growth substrate is useful and is sometimes essential for unique sensor assembly.

FIG. 14 illustrates Nano or micro bridge width control of In₂O₃ type semiconducting film. FIG. 14(a) shows a wider bridge array, FIG. 14(b) shows an intermediate width bridge array, FIG. 14(c) shows a nanoscale narrower bridge array. An array of narrower bridge such as nano-dimension-width bridges of less than 50 nm provides various advantageous properties for the biosensor.

FIG. 15 illustrates controlling the number of bioreceptors (sensor molecules such as aptamers or polymerase enzyme molecules) attached onto the In₂O₃ semiconductor microbridge or nanobridge surface. Such biosensors are useful for DNA sequencing, disease-causing antigen, antibody levels, etc.

FIG. 16 illustrates a semiconductor bridge array physically secured by metal electrode array on top. (a) Many bioreceptors attached onto the nanobridge surface. FIG. 16(b) illustrates circular open holes patterned on the dielectric ceramic or polymer mask. Only these exposed areas allow the bioreceptor (sensor) molecules to attach to the semiconductor. (c) Only three sensor molecules are allowed to attach per each bridge onto the unmasked areas of the nanobridge.

FIG. 17 illustrates exemplary shapes of the biomolecule-blocking dielectric mask. FIG. 17 illustrates (a) circular openings in the mask, FIG. 17(b) illustrates elongated slot shape opening in the mask more conveniently allowing specific sites in all bridges in a nanobridge array to be available for biosensor molecule attachments.

FIG. 18 illustrates a DNA or genome sequencing application of ultrathin oxide FET (field effect transistor) molecular biosensor in a microfluidic chamber (not shown), detecting the nucleotide attachment by electronic signals. (The location-dictating-mask for selected-site attachment of polymerase type sensor molecule is not shown.)

FIG. 19 illustrates an exemplary FET molecular biosensor in a microfluidic chamber environment (not shown), based on oxide semiconductor nanobridges for detection of various analytes including COVID-19 virus, other coronavirus types, disease-inducing or epidemic-causing virus, or other microorganisms, proteins or DNAs in forensic analysis, sensing of environmental pollutants (organic or inorganic) or DNA analysis. FIG. 19(a) shows peptide based sensor molecules with primers, FIG. (b) shows an aptamer based, primer-added sensor molecules. (The sensor molecule location-dictating-mask is not shown.)

FIG. 20 illustrates a wearable or instant-analysis FET molecular biosensor for biofluid analysis (e.g., from sweat or blood droplet) in a non-microfluidic chamber environment, based on oxide semiconductor nanobridges for detection of various analytes including metabolic status, hormone levels, etc.

FIG. 21 illustrates an exemplary DNA memory device utilizing oxide-semiconductor-nanobridge based sequencing analysis biosensor bridge structure. FIG. 21(a) shows tethered array of encoded (memory written) DNA fragments periodically positioned on a substrate, FIG. 21(b) shows transferred and anchored, width-reduced nanobridge array (or other semiconductor nanobridge array in general,) approaching information stored DNA array being released, FIG. 21(c) shows DNA templates are taken up by polymerase array and the completed sensor array is moved away for DNA sequencing to read the recorded memory information. (Microfluidics chamber not shown). Massively parallel DNA written information array in combination with massively parallel nanoribbon bridge reader array allows ultrafast, “random-access-enabled” DNA memory retrieval. Several different configurations of tethered DNA memory array, with various methods of tethering and releasing, or nano sensor access schemes may be made to maximize the memory processing capability.

FIG. 22 illustrates electrical resistivity control of In₂O₃ thin films during RF sputtering by increased oxygen partial pressure in the sputtering chamber. The RF power used was 200 Watt, the total sputtering time was 10 minutes, the substrate was SiO₂ coated Si, and the substrate temperature was the room temperature (about 20° C.). The resultant In₂O₃ film was about 100 nm ±10 nm thick.

FIG. 23 shows an exemplary CMOS platform nano-bio sensor array based on nanobridge structure array onto which biosensor molecules are conjugate attached. FIG. 23(a) is an example of about 16,000 biosensor CMOS chip array according to one embodiment of the disclosure, FIG. 23(b) is an example nano-bio FET sensor structure, FIG. 23(c) illustrates an exemplary mechanism of electrical signal generation on binding vs unbinding of target analyte molecules onto the biosensor.

FIG. 24 illustrates Fabrication of Indium Oxide (In₂O₃) nanoribbons of different dimensions on CMOS sensor chips according to one embodiment of the disclosure.

FIG. 25 illustrates a zoomed-out view (electron microscopy) of In₂O₃ nanoribbons ribbon FET sensors on a CMOS chip with 4-pixel quad electrode array.

FIG. 26 illustrates typical traces from the positive and negative targets from three separate chips whereby an aptamer specific to His-tag on a protein is attached to the Indium Oxide nanoribbons that was functionalized with silanes. The response curve from various chips are also shown by plotting the cumulative sum of the change in mean current from phase to phase against the target concentration. The electrical measurements of In₂O₃ nanobridge biosensor signals for individual sensor devices are shown: for buffer solution only without any target molecules (FIG. 26(a), binding assay signal with intentionally added wrong target biomolecules (FIG. 26(b)), and binding assay signal with correct target biomolecules FIG. 26(c). The data in the FIG. 26(d) represents a plot of aggregate biosensors (multiple biosensors) binding assay signals vs. concentration of the analyte target molecules, with electrical signal error bars included.

FIG. 27 illustrates traces from the positive and negative targets on the same chip are shown, whereby an aptamer specific to Hepatitis B surface Antigen (HbsAg) is attached to the Indium Oxide nanoribbon nanobridge that was functionalized with silanes. Also, the response curve from various targets is shown by plotting the cumulative sum of the change in mean current from phase to phase against the target concentration. The electrical measurements of In₂O₃ nanobridge biosensor signals for individual sensor devices are shown; FIG. 27(a) for buffer solution only without any target molecules, FIG. 27(b) binding assay signal with intentionally added wrong target biomolecules, and FIG. 27(c) binding assay signal with correct target biomolecules. The data in FIG. 27(d) represents a plot of aggregate biosensors (multiple biosensors) binding assay signals vs concentration of the analyte target molecules. The target concentration numbers (in picomolar) for the HbsAg target molecules are also shown toward the right side of plot (d).

FIG. 28 illustrates resistivity increase in nanobridge of In₂O₃ and related metal-nonmetal semiconductors by porous structure. FIG. 28(a) shows In₂O₃ rectangular nanobridge with near full density, positioned under the source-drain metallic electrodes, FIG. 28(b) shows similarly positioned but porous nanobridge with higher electrical resistivity.

FIG. 29 illustrates resistivity increase in nanobridge of metal-nonmetal compound (e.g., In₂O₃ nanobridge) by intentional inclusion of insulating ceramic for volume dilution; FIG. 29(a) shows full density In₂O₃ nanobridge positioned under the source-drain metallic electrodes, FIG. 29(b) shows similarly positioned In₂O₃ nanobridge but comprising embedded insulating-islands.

FIG. 30(a) illustrates large size anchoring islands (e.g., >30 nm equivalent diameter). FIG. 30(b) shows small, size-limited nanoscale islands (e.g., 5 -15 nm). Large diameter anchoring islands, FIG. 30(a), may cause an undesirable, multiple polymerase molecule binding, as shown in FIG. 30(c). Size-limited, nano-island array, shown in FIG. 30(b), with the equivalent diameter of each anchoring island comparable to the average diameter of polymerase (about 3-10 nm), aptamer, DNA or peptide of similar regime of sizes, essentially ensures the attachment of a single molecule bioreceptor sensor molecule as shows in FIG. 30(d).

FIG. 31 illustrates the use of noble metal anchoring islands for precise control of number and location of bioreceptor molecule attachment (such as aptamers, polymerase enzyme molecules, DNAs, peptides). FIG. 31(a) shows uncontrolled, multiple bioreceptor sensor molecules attached onto each In₂O₃ bridge surface. FIG. 31(b) shows two noble metal anchoring islands intentionally placed on In₂O₃ bridge surface, FIG. 31(c) shows only two sensor molecules (e.g., polymerase enzyme) are allowed to attach onto the two anchoring islands, with strong covalent binding, FIG. 31(d) shows only a single sensor molecule (e.g., polymerase enzyme) is allowed to attach onto the single anchoring island (most ideal for DNA or genome sequencing).

FIG. 32 illustrates nanofabricated nano-island array. FIG. 32(a) shows 10 nm diameter Si nano-island array by electron-beam lithography, FIG. 32(b) shows 15 nm diameter Ni metal island array by e-beam lithography of 33 nm diameter, a few nm thick Ni film disk array, followed by ball-up annealing at about 400° C. to intentionally reduce the island diameter to 15 nm.

FIG. 33(a) shows sharp Si nanocone array (e.g., about 2 nm tip radius) by etching and oxidation repeat process, FIG. 33(b) shows 5-8 nm diameter tapered pillar array for nanoimprint mold fabrication using Si nanofab process.

FIG. 34 illustrates nanoimprinting process to form a sub-10 nm metal anchoring island array using a about 5-8 nm diameter tip arrayed imprinting mold according to one embodiment of the disclosure.

FIG. 35 illustrates multiplexing biosensing using multiple regions of analyte droplet solutions (e.g., for separate assay or sequencing with different patient's samples, or the same patient's samples for different types of analysis).

FIG. 36 illustrates the use of patterned hydrophobic mask layer on hydrophilic substrate to create spaced-apart analyte solution droplets array.

FIG. 37 illustrates an exemplary subdivided platform to allow multiplexing biosensor analysis. Different compartments may contain different patient samples, or the same patient's samples but for different types of analysis).

The figures presented herein are exemplary and illustrative in nature and are not intended to limit the scope of the disclosed principles.

### DETAILED DESCRIPTION

Manipulations and analyses of biomolecules such as for DNAs, peptides, proteins, and genomes, as well as detection of antigens such as viruses (like SARS-COV-2, the COVID-19 virus), bacteria and other disease-causing micro/nano organisms, and nutrients, metabolic or hormone-related parameters, sensing of toxic compounds or molecules, have received much attention for healthcare and other beneficial purposes. Such detections and analyses are therefore important with respect to potential applications in precision medicine, drug discovery, environmental monitoring, basic research or nanotechnology.

DNA is a particularly important biomolecule both as an analyte and a probe, due to its fundamental role in biology. The seminal work of Maclyn McCarty and Oswald T. Avery in 1946, (see, “Studies On The Chemical Nature Of The Substance Inducing Transformation Of Pneumococcal Types II. Effect Of Desoxyribonuclease On The Biological Activity Of The Transforming Substance,” The Journal of Experimental Medicine 83(2), 89-96 (1946)), demonstrated that DNA was the material that determined traits of an organism. The molecular structure of DNA was then first described by James D. Watson and Francis H C Crick in 1953, (see a published article, “Molecular structure of nucleic acids.”, Nature 171,737-738 (1953)), for which they received the 1962 Nobel Prize in Medicine. This work made it clear that the sequence of chemical letters (bases) of the DNA molecules encode the fundamental biological information. Since this discovery, there has been a concerted effort to develop means to experimentally measure this sequence. The first method for systematically sequencing DNA was introduced by Sanger, et al in 1978, for which he received the 1980 Nobel Prize in Chemistry. See an article, Sanger, Frederick, et al., “The nucleotide sequence of bacteriophage φX174.” Journal of molecular biology 125, 225-246 (1978).

DNA sequencing techniques for genome analysis evolved into utilizing automated commercial instrument platform in the late 1980's, which ultimately enabled the sequencing of the first human genome in 2001. This was the result of a massive public and private effort taking over a decade, at a cost of billions of dollars, and relying on the output of thousands of dedicated DNA sequencing instruments. The success of this effort motivated the development of a number of “massively parallel” sequencing platforms with the goal of dramatically reducing the cost and time required to sequence a human genome. Such massively parallel sequencing platforms generally rely on processing millions to billions of sequencing reactions at the same time in highly miniaturized microfluidic formats. The first of these was invented and commercialized by Jonathan M. Rothberg's group in 2005 as the 454 platform, which achieved thousand fold reductions in cost and instrument time. See, an article by Marcel Margulies, et al., “Genome Sequencing in Open Microfabricated High Density Picoliter Reactors,” Nature 437, 376-380 (2005). However, the 454 platform still required approximately a million dollars and took over a month to sequence a genome.

The 454 platform was followed by a variety of other related techniques and commercial platforms. See, articles by M. L. Metzker, “Sequencing Technologies—the Next Generation,” Nature reviews genetics 11(1), 31-46 (2010), and by C. W. Fuller et. al, “The Challenges of Sequencing by Synthesis,” Nature biotechnology 27(11), 1013-1023 (2009). This progress led to the realization of the long-sought “$1,000 genome” in 2014, in which the cost of sequencing a human genome at a service lab was reduced to approximately $1,000 and could be performed in several days. However, the highly sophisticated instrument for this sequencing cost nearly one million dollars, and the data was in the form of billions of short reads of approximately 100 bases in length. The billions of short reads often further contained errors so the data required interpretation relative to a standard reference genome with each base being sequenced multiple times to assess a new individual genome.

Thus, further improvements in quality and accuracy of sequencing, as well as reductions in cost and time are still needed. This is especially true to make genome sequencing practical for widespread use in precision medicine (see the aforementioned article by Fuller et al.), where it is desirable to sequence the genomes of millions of individuals with a clinical grade of quality. While many DNA sequencing techniques utilize optical means with fluorescence reporters, such methods may be cumbersome, slow in detection speed, and difficult to mass produce to further reduce costs. Label-free DNA or genome sequencing approaches provide advantages of not having to use fluorescent type labeling processes and associated optical systems, especially when combined with electronic signal detection that may be achieved rapidly and in an inexpensive way.

Certain types of molecular electronic devices may detect the biomolecular analytes such as DNAs, RNAs, proteins, antigens, and nucleotides by measuring electronic signal changes when the analyte molecule is attached to the circuit comprising a pair of conductive electrodes. Such methods are label-free and thus avoids using complicated, bulky and expensive fluorescent type labeling apparatus. These methods may be useful for lower cost sequencing analysis of DNA, RNA and genomes. While current molecular electronic devices may electronically measure molecules for various applications, they lack the reproducibility as well as scalability and manufacturability needed for rapidly sensing many analytes at a scale of up to millions in a practical manner. Such highly scalable methods are particularly important for genome sequencing applications, which often need to analyze millions to billions of independent DNA molecules. In addition, the manufacture of current molecular electronic devices is generally costly due to the high level of precision needed.

The disclosed embodiments provide new and improved sequencing apparatus and associated sensor configurations and methods using advanced elongated bridge structures or related semiconductor nanoribbons or nanowires, which provide reliable DNA genome analysis performance and detection of various analytes (e.g., label free) including proteins, DNA, RNA, small molecules, viruses, bacteria, pathogens or chemical pollutants. The disclosed apparatus and devices are more easily amenable to scalable manufacturing. The disclosed structures are also useful for DNA-based large-capacity information storage devices including archival or randomly accessible memory (RAM) and logic devices.

DNA data storage is a process of digital encoding and decoding binary data, to and from synthesized or duplicated DNA strands. For example, the binary code information storage of (00), (01), (10) and (11) may be replaced by various arrangements of oligonucleotides (A, C, G, T). As is well known, DNA molecules are genetic blueprints for living organisms, and the information stored in DNA is known to last more than 10,000 years in favorable environments. With its huge capacity (many orders of magnitude larger information density than what is possible with current technology) to store enormous amount of information in very small space, DNA storage could be the answer to a modern era problem of too much information that needs to be stored, e.g., on the order of hundreds of zettabytes every year in the near future. Currently available information storage capability including magnetic disk, tape, optical or other related technologies may cover only a fraction of such a needed capacity.

While substantial progress has been made in DNA information storage in recent years, cost effective data storage techniques for practical applications are yet to be achieved. For efficient retrieval of stored information, the encoded DNA nucleotide arrangements need to be decoded by, for example, reading (or sequence analysis). A fast, economic method of reading the encoded DNA information is essential for the success of DNA based data storage. This invention also provides new methods and device structure to enable such progress.

In the case of biosensors using electrical signals as the basis of analyte detection, the SNR of detection is often dependent on the baseline electrical resistance (or impedance). For enhanced SNR of electrical or electronic based biosensors, one way of increasing sensitivity according to the disclosed embodiments is to reduce the sensor dimensions, such as the bridge connecting two electrodes, on which the bioreceptor sensing molecules such as enzymes (e.g., DNA polymerase, CRISPR Cas, antibodies, aptamers, peptides, or DNA oligos, or cells) are placed, with the sensing events comprising the singular or multiple attachment of analytes onto the bioreceptor molecule(s). As the analyte attachment event induces a quite small change in electrical conductivity of the bridge, which is to be detected, it is important to maintain a reasonably low electrical conductivity in the bridge onto which the sensing bioreceptor biomolecule(s) are attached. The change in electrical conductivity in such sensors are often measured by, e.g., detecting changes in the amount of electrical current, voltage, electrical resistance (or impedance), or capacitance, for example, in the form of field effect transistors.

For the purpose of increasing SNR in such bioreceptor-based molecular sensors according to one embodiment of the disclosure, the bridge dimension is made small. This may be done, for example, by (i) using a pre-made (pre-fabricated), small diameter bridge such as DNA, RNA, peptide, or (ii) utilizing a small dimension (thin and narrow) inorganic semiconductor bridge such as metal oxide, metal nitride, metal carbide, metal phosphide semiconductors, and other materials. If the sensor bridge is made even thinner and/or narrower, the baseline electrical resistance (or impedance) will become higher, and hence the small electrical signal (or the change in electrical conductivity, measured by a change in electrical current, voltage or capacitance) induced by analyte attachment event onto the bioreceptor will not be lost in the high current background that otherwise results from a low resistance electrical circuit.

According to the principles provided herein, novel biosensors with label-free, improved electrical sensing capabilities via structural and dimensional control are disclosed, in particular, with the dimension of the bridge platform that supports one or multiple bioreceptor sensor molecules altered for enhanced SNR, with their locations on the nanobridge surface precisely controlled and pre-determined using mask structures that expose only the desired sites for probe attachment. The devices in this invention utilize various bioreceptor sensor molecules such as one or more enzymes (e.g., DNA polymerase), cells, antibodies, aptamers, peptides or DNAs. The disclosed devices are useful for various biomedical or non-biomedical applications such as monitoring of diseases for point-of-care diagnostics, detection of analytes (including but not limited to—disease-inducing or epidemic-causing bacteria, virus or other micro-organisms, nucleotides in DNA analysis and genome sequencing, proteins or DNAs in forensic analysis), sensing of environmental pollutants (organic or inorganic), drug development (organic or inorganic), and information storage (e.g., using an array of DNAs, RNAs, proteins, DNA origami structures, antibodies, aptamers, enzyme molecules).

Sensor structures having many probe molecules with random attachment of sensor onto the source-drain bridge(s) or channel(s) are known in the art and have proven useful for detection of various analyte molecules and disease-causing pathogens. However, the art does not have a precise control of the sensor molecule positions, numbers or qualities.

It is therefore desirable to have an advanced, more reliable biosensor structures with precise sensor molecule control in terms of the molecule position, number, quality, and periodic or patterned arrangement, as is disclosed in this invention. The disclosed principles provide well designed array of thin and width-reduced, bridge-configured elongated nano-dimension semiconductor wire or ribbon as a base platform to carry a bioreceptor sensing molecule, optionally and preferably with a sensor molecule position-, number-and distribution-restricting bridge structure.

The biomolecule probe sensor array comprising an array of microbridges or nanobridges, the biosensor device may advantageously be a single molecule biosensor per semiconductor bridge. However, for redundancy and other quality control purposes, as well as for consideration of probe molecule attachment efficiency, a two-molecule biosensor per bridge, a three-molecule biosensor per bridge, or up to ten-molecule biosensor per bridge may be utilized. For some applications, having two or more different biosensor molecules attached onto the same nanobridge is useful, if those biosensor molecules are not identical, in which case each biosensing bridge may perform sensing of more than one type of analytes.

In the case of multi-biomolecule probe sensor, the biomolecule attachment sites may be a periodic arrangement or specified spatial pattern. The spacing between neighboring sites may be controlled and kept small to achieve a high density of probe molecules and that probe molecules are uniformly distributed over the functionalized portion of the bridge. This may reduce variability in the resulting modulation of the bridge current or conductivity. This may also ensure that the probe molecules are spaced out to reduce or eliminate unwanted probe-probe molecular interactions, and such spacing out of probes can ensure probe molecules more accessible to interact with the target analytes and thereby increase sensitivity.

Such an electronic biosensor with high SNR is therefore desirable, for genome sequencing or for detection of disease-inducing virus (including SARS virus, coronavirus including COVID-19 virus, and other organic analytes or pollutants) without introducing complicated fluorescence imaging methodologies. Such electrical sensors without cumbersome optical sensing arrangements can be produced using, e.g., field effect transistor (FET) configuration using either single gated, multi-gated or non-gated structures.

The analyte attachment event may be viewed as a gate event that influences the electrical behavior of the underlying semiconductor bridge. This invention discloses structures, methods and applications of dimension-controlled high SNR electrical biosensors. As the invention devices can be obtained by varying the details of the sensor structures and methods of preparations, various embodiments of structures and techniques are described in detail as below. Each of the disclosed embodiments is provided to illustrate an application of the disclosed principles. As a result, the disclosed embodiments are non-limiting and exemplary.

### Embodiment 1—Ultrathin Oxide Semiconductor Nanobridge Positioning

In the FET type biosensors, the semiconductor channel (or bridge) connects a source electrode and a drain electrode, and the change in the electrical characteristics of the channel semiconductor upon biomolecular reactions of attached biosensor molecules is measured to detect such events. The source and drain electrodes are typically made of highly conductive metallic materials such as Au, Pt, Pd, Ag, Ru, Rh, Ir, Cu, Ni or their alloys.

In certain embodiments, biosensors comprising one, or multiple, or an array of ultrathin, generally sub-20 nm thick, preferably sub-10 nm thick, preferably in the range of 5 nm down to 0.1 nm (or atomic monolayer), semiconductor ribbon bridges, with a width that is micrometer scale or preferably sub-micrometer, preferably sub-50 nm, more preferably sub-20 nm in width, and most preferably in the range of 10 nm down to 1 nm in width, are utilized to connect two metal electrodes of source and drain. One, or multiple, or a patterned array of biosensor/bioreceptor molecules (such as a polymerase enzyme molecule sensor, an aptamer molecule sensor, a DNA-oligo sensor, and so forth) are placed on the semiconductor micro-bridge or nano-bridge to perform electronic sensing reactions when analyte molecules or components are attached onto the biosensor molecules and thereby produce electronic signal pulses. It should be noted that the dimensions provided herein may be an approximation.

In one embodiment, the nanoribbon bridge may not be of a uniform width and may instead have a width constriction at the location or locations where the probe molecule is attached, so that the local current gating effect of probe molecule binding is enhanced, due to the greater impact on current flow through the local constriction in the ribbon. In preferred embodiments, the nominal local ribbon width may be, for example, in the range of 20 nm to 5 um, more preferably 20 nm to 200 nm, while the constriction narrows to the range of 5 nm to 50 nm, more preferably 1 nm to 20 nm in the vicinity of the probe attachment site.

The semiconductor nanoribbon bridges, e.g., made of metal-oxide semiconductors, metal-nitride semiconductors, metal-carbide semiconductors, or metal-phosphide semiconductors, according to the invention, can be positioned above the metal electrodes, below metal electrodes, or above metal electrodes with the bridge outer ends anchored to enhance physical and electrical contacts of the semiconductor bridges to the electrodes.

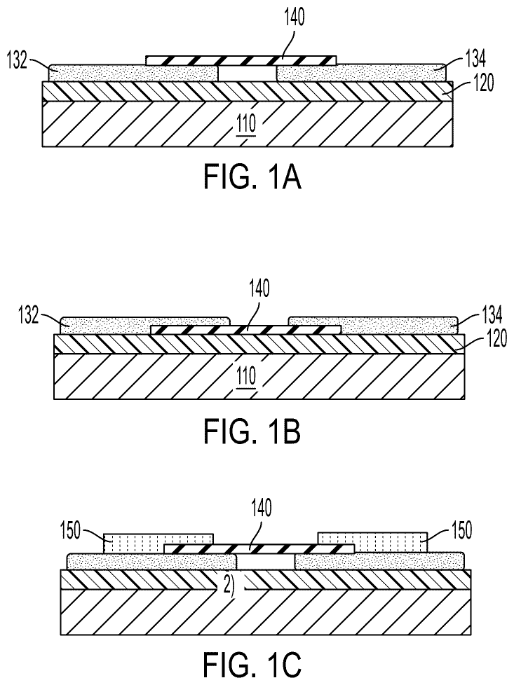

Possible variations of such semiconductor devices 100 bridge position are illustrated in FIG. 1, which describes the formation of ultrathin semiconductor nanobridge array for biomolecular sensors based on metal oxide, metal carbide, metal nitride or other metal-nonmetal compound material. FIG. 1 illustrates substrate 110 supporting dielectric layer 120 upon which rest metal electrodes 132 and 134. Substrate 110 may comprise SiO₂/Si, sapphire (Al₂O₃), LaAlO₃, MgO or various other substrates with optional semiconductor base for gated FET applications. Ultrathin, narrow oxide semiconductor nanobridge 140 may comprise one or more of carbide, nitride, phosphide or semiconductor material. Metal electrode pairs 132, 134 define the source and the drain electrodes and may comprise Pt, Pd, Au, Ru, Rh or alloys. The metal electrodes may be optionally polarized. In FIG. 1(a), nanobridge 140 is on top of metal electrodes 132, 134. In FIG. 1(a), nanobridge 140 may be patterned on top of source-drain metal electrode pair. The nanobridge or its array can also be prepared on a separate substrate, then stamp transferred on top of source-drain metal electrode pair, wherein inherent van der Waals force of nanolayered materials are utilized for anchoring.

In FIG. 1(b), nanobridge 140 is secured under metal electrodes is illustrated, as the metal electrodes are deposited after the nanobridge is placed first. The ultrathin, narrow oxide semiconductor nanoribbon bridge may be formed when the semiconductor thin film is placed first (e.g., direct deposition) and patterned or pre-patterned.

In FIG. 1(c), a nanobridge on top of metal electrodes is shown, but with an enhanced nanobridge adhesion by an added anchoring structure 150 on the transfer deposited nanobridge surface. Here, an ultrathin, narrow oxide semiconductor nanoribbon bridge is first placed. Then anchoring structure 150 is added to ensure the physical stability of the nanobridge. A conventional stamp transfer or nano-imprinted polymer ribbon array or polymer picture-frame, stamp transferred thin dielectric layer or picture-frame can be used. Each anchoring structure 150 may optionally cover many nanobridge arrays.

The oxide-based FET biosensor devices utilizing biomolecular sensors (such as utilizing one or more of DNA polymerase enzyme molecules) are particularly applicable to genome sequencing, detection of disease-causing virus including COVID virus or bacteria, drug discovery, DNA memory devices and other biotech applications.

Oxide base semiconductors, can be either n-type semiconductors (such as ZnO, In₂O₃, SnO₂, (In,Sn)₂O₃ (ITO type), NbO₂, In—Ga—O (IGO), In—Zn—O (IZO), In—Ga—Zn—O (IGZO), In—Al—Zn—O (IAZO), TiO2, WO₃, and various doped versions, or they can be p-type semiconductors (such as CuO, Cu₂O, NiO, spinel oxides, CuMO₂ (M=Al, Ga, In, such as CuInO₂, CuAlO₂, CuGaO₂, CuSrO₂, SrCu₂O₂), LaCuOS, LaCuOSe, with various types and concentrations of dopants added as needed.

In certain embodiments, the conductance and resistance increase for higher SNR, the desired width of the oxide semiconductor nanoribbon-shaped nanobridge is, e.g., less than 200 nm in width, preferably less than 50 nm, more preferably less than 20 nm, even more preferably less than 10 nm. The desired thickness of the oxide semiconductor nanoribbon-shaped nanobridge is, e.g., less than 50 nm, preferably less than 20 nm, more preferably less than 10 nm, even more preferably less than 2 nm.

As it is desired to have the cross-sectional area of the nanobridge minimized, the desired range of cross-sectional area may be less than about 10,000 (nm)², less than 1,000 (nm)² , less than 200 (nm)² or less than 50 (nm)².

In order to minimize the electrical conductance of the nanobridge, the desired length of the oxide semiconductor nanoribbon may be, for example, less than 50 um, less than 1 um, less than 200 nm, or less than 100 nm.

In order to achieve such nano-width ribbons, a desired patterning processing is either e-beam lithography, EUV lithography, laser lithography, nanoimprint lithography type high resolution processing steps. Photolithography alone, a use of shadow mask or a stencil mask is to be avoided as such a process produces too coarse nanobridge structure and associated low SNR on biosensing.

Carbide semiconductors (e.g., SiC, GeC) or nitride semiconductors (e.g., GaN, AlGaN, InGaN, GaInAsN) can also be utilized. Phosphide-based semiconductors such as InP, or sulfide-based semiconductors such as CdS, In₂S₃, SnS₂, CuS₂, ZnS, MoS₂ or WS₂ can also be used.

The properties of semiconductor bridge materials can optionally be improved by doping, e.g., n-type doping or p-type doping, e.g., by diffusional addition of doping element, by ion implantation with desired implantation ion acceleration energy and desired level of doses, with optional post-annealing, or other means. Such doping can be useful for semiconductor bandgap adjustment or for enhancement of charge carrier concentration or mobility. Ion implantation doping of micro/nano bridge structure can be performed either directly on the finished micro/nano bridge connecting the source-drain metal electrodes or performed first on the semiconductor film on a separate substrate followed by patterning and transfer deposit (e.g., using elastomeric stamp or adhesive stamp) onto the device (having source-drain metal electrode pair array) to form a bridge array with doped semiconductor micro/nanobridges. The transfer deposit process can be done with either an unpatterned film (followed by patterning during or after the electrode pair array construction) or a pre-patterned film having a desired micro/nano ribbon configuration. Such ion implantation improvement of micro/nano bridge can also be carried out on other non-oxide, 2D type semiconductor films, e.g., graphene (to open the bandgap), transition metal dichalcogenide (TMD) film such as MoS₂, WSe₂, and so forth.

The device substrate can be SiO₂/Si, sapphire (Al₂O₃), LaAlO₃, MgO, or various other dielectric substrates with optional semiconductor base for gated FET biosensor applications. Such oxide nanobridge can be either in-situ deposited on device (substrate) itself, or premade on a separate substrate and nanopatterned into a nanoribbon array with optional follow-up processing of annealing heat treatment, ion implantation or other doping process, nanopatterning into narrow ribbons by nanofab processing of lift-off, e-beam lithography, nanoimprint lithography, etc. followed by elastomeric stamp transfer, adhesive tape transfer or thermally releasable adhesive transfer.

The deposition of oxide semiconductor can be achieved, e.g., by physical vapor deposition (such as DC or RF sputtering, evaporation, ion beam deposition), chemical vapor deposition (CVD), electrochemical deposition, atomic layer deposition (ALD) like In₂O₃ semiconductor using trimethyl indium source, precursor deposition and post-treatments. Alternatively, a metallic component such as indium or zinc can be deposited first and nanopatterned, then the nanoribbons so obtained can be oxidized by reactive process or thermal oxidation process, or the metal film can be oxidized first, then nanopatterned. Nanoribbons of such oxide semiconductors can be obtained on the biosensor device by nanopatterning such as e-beam lithography, nanoimprint lithography, extreme UV (EUV) photolithography, and other nanopatterning methods. An alternative technique may be to prepare the nanoribbons of oxide semiconductor film on a separate substrate followed by stamp transfer or adhesive transfer onto the surface of the device electrode array.

Adhesion stability of stamped or other transfer-deposited nanoribbon nanobridges on source and drain electrode surface can be further improved by placing an anchoring structure (e.g., 0.5-50 nm thick) on the outer edges of transferred nanobridge, e.g., using a dielectric layer like Al₂O₃, a polymeric layer like PMMA (polymethylmethacrylate) or HSQ (hydrogen silsesquioxane), or a metallic layer. The anchoring structure can be processed on the service itself, or more conveniently stamp transferring or nano-imprinting of a premade polymer ribbon array or polymer picture-frame anchor layer (such as premade by atomic layer deposition (ALD), sputtering, evaporating, exfoliation of e.g., Al₂O₃, SiO₂, Cr, Ni, Au, Pt, Pd, etc). Each anchoring structure may conveniently cover many nanobridges or arrays simultaneously.

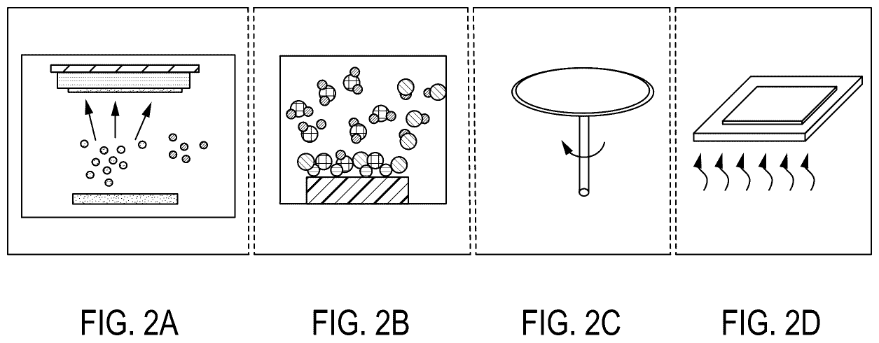

FIG. 2 schematically illustrates various methods that can be utilized to prepare an oxide semiconductor thin film such as an In₂O₃ film for fabrication of nanobridge biosensors according to certain disclosed embodiments. Specifically, FIG. 2(a)-2(d) show various methods that can be utilized to prepare semiconductor thin films, such as In₂O₃ films for the disclosed biosensors. The possible methods are illustrated, which include physical vapor deposition as shown in FIG. 2(a), chemical vapor deposition (CVD), atomic layer deposition (ALD) using trimethyl indium or other indium compounds, electrochemical deposition, and so forth as shown in as shown in FIG. 2(b). In FIG. 2(c), liquid phase precursor solution is spin coated followed by baking to convert into a desired oxide phase. In FIG. 2(d) a metallic component deposition is carried out first, followed by oxidation to convert the metal film into an oxide film as shown in FIG. 2(d).

The sputtering process (FIG. 2(a)) to form an oxide film such as In₂O₃ can be performed by e.g., RF sputtering, reactive sputtering, etc. In some occasions, amorphous In₂O₃ is preferred to crystalline material if grain boundary and other defects are to be avoided. One of the useful techniques for forming amorphous oxide is to use a low substrate temperature by utilizing a cooling stage (e.g., 0° C. to as low as −196° C., liquid nitrogen temperature) to minimize crystallization. The In₂O₃ may be an n-type semiconductor. If further doping is needed, the sputtering target composition can be modified, or a reactive sputtering is utilized to add dopants. Post-deposition ion implantation can also be utilized for doping.

In₂O₃ type thin films can also be obtained by spin coating a precursor solution (FIG. 2(c), aqueous or organic, (e.g., indium nitrate, chloride, sulfate based, etc.), optionally adding organic binder type or surfactant material to control the viscosity and enhance uniformity of spin coating, e.g., adding a small quantity of polyvinyl alcohol. The spin coated film is then baked at 100-600° C., preferably 200-300° C. to form a thin oxide semiconductor layer e.g., 1-20 nm thick In₂O₃).

If a metallic component film layer (e.g., indium film layer, physical, chemical or electrochemically deposited) is deposited first (and optionally nanobridge patterned), it can then be oxidation treated to form e.g., 1-20 nm thick In₂O₃. The oxidation treatment can be carried out at room temperature or at a high temperature, using for example, oven heating, microwave heating, resistive heating, IR heating, oxygen plasma treatment, chemical or electrochemical oxidation, and so forth, optionally using oxygen or ozone partial or full gas atmosphere. For nitride, carbide, phosphide semiconductor formation from the pre-deposited metal, these can similarly be achieved by nitriding, carburizing, phosphiding type post-treatments to the metallic film (either a wide-area film or pre-nanopatterned ribbon array film), respectively.

### Embodiment 2—Thickness Reduction and Width Reduction of Ultrathin Oxide Semiconductor Nanobridges

Thinner and narrower oxide semiconductor nanobridges are generally more desirable to improve the semiconductor or FET properties, and to increase the SNR as the electrical characteristics of the reduced volume nanobridge tends to accentuate the electronic pulse signal from the biosensing event, instead of burying the small signal.

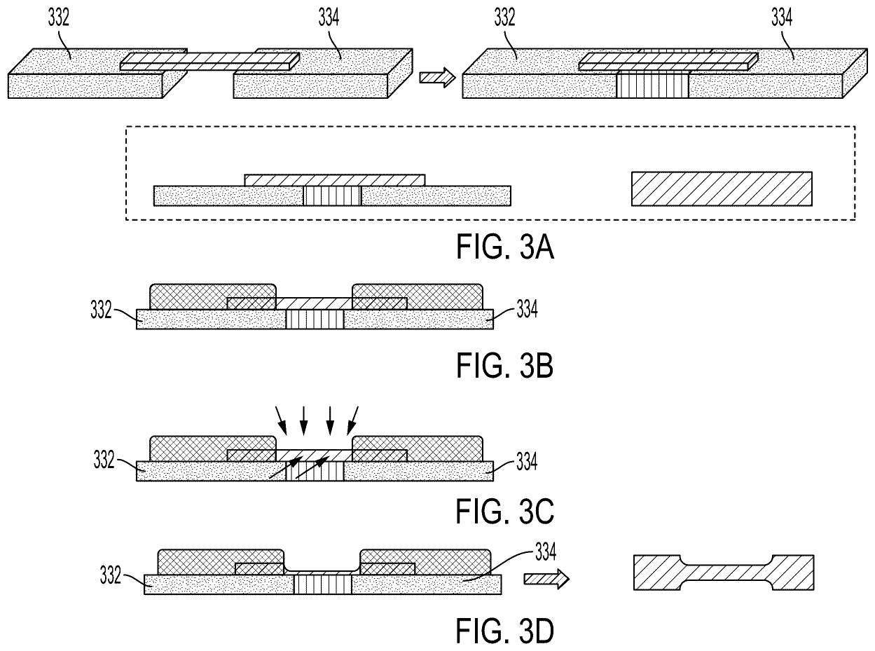

FIG. 3 schematically illustrates an exemplary process according to one embodiment of the disclosure. Specifically, FIG. 3 shows steps (a) through (d) for thickness and width reduction of ultrathin semiconductor nanobridge using a chemical etch or plasma etch for erosion from the side edges and from the top of the nanoribbon. As illustrated in FIG. 3, thickness reduction and width reduction of ultrathin semiconductor nanobridge such as an oxide ribbon nanobridge (only one nanobridge shown in an array structure) using a chemical etch or plasma etch for erosion from side edges and from the top of the nanoribbon. In FIG. 3(a), a nanobridge placement is carried out (e.g., by stamp transfer or by in-situ deposition/nanopatterning on device itself). In FIG. 3(b), an etch barrier is added to enable a selective etching of nanobridge top and side edges. In FIG. 3(c), thinning and narrowing process is performed for the nanobridge such as an oxide semiconductor using a plasma etch, ion etch, chemical etch, electrochemical etch, and so forth. In FIG. 3(d), cross-sectional and top views of thinner and narrower oxide nanobridge after selective etch of barrier-free, exposed region of nanobridge are schematically shown.

For nanobridge width reduction and thickness reduction process as illustrated in FIG. 3, the nanobridges of ultrathin oxide semiconductor are first positioned on a substrate, either suspended on a pair of metal electrodes or on planarized electrode surface (e.g., using a polymer dielectric, or HSQ negative resist filling, e-beam exposure with a post heat treatment to convert to a SiO₂ type filler material, followed by planarization, e.g., using a RIE etch to make the filler height the same as or comparable to the metal electrode height.)

The desired amount of cross-sectional area reduction in the nanobridge by chemical, electrochemical or plasma ion etch may be at least by 10%, at least 30%, at least 50%. The nanobridge width reduction can be carried out, e.g., from 20-100 nm to 10-50 nm, to 5-20 nm width, and the nanobridge thickness reduction can be performed, e.g., from 10 nm to 1-3 nm, to 0.5-1 nm, unless the semiconductor ribbon thickness is already sufficient small.

Plasma etch (like RIE etch), ion etch, or chemical etch (like HF base acid) from side edges, or electrochemical etch can be used for the width reduction and thickness reduction. The etch barrier can be utilized to prevent (or to minimize) the etching of the end portions of the semiconductor nanobridge, while the exposed middle section gets etched further by plasma etch like reactive ion etch (RIE), ion etch, chemical etch, high temperature etch, ion-implant-enhanced etch, etc. for thinning (and narrowing) of the nanobridge.

For nanobridge structures such as those comprising In₂O₃ oxide semiconductor, which may be positioned underneath the source-drain electrode pairs (such as Pt, Au, Pd, Ir, Ru, Re), the metal electrode strip present above the nanobridge can be utilized as a mask. Part of the top surface and side edges of the In₂O₃ nanobridge may be etched away to make the nanobridge cross-section smaller. For example, a slow and controlled chemical etching of the In₂O₃ nanobridge, e.g., using a diluted acid at low temperature can be used to preferentially etch In₂O₃ over e.g., Pt electrode. Alternatively, a RIE (reactive ion etch) process with a selected ion etch species more active for In₂O₃ material than the electrode material like Pt, the desired cross-section reduction of the In₂O₃ nanobridge can be accomplished.

### Embodiment 3—Further Dimensional Reduction of Semiconductor Nanobridge Using an Erodible Resist Mask

In the case of biosensors using electrical signals as the basis of analyte detection, the SNR of detection is often dependent on the baseline electrical resistance (or impedance). For enhanced SNR of electrical or electronic based biosensors, one way of increasing the sensitivity is to reduce the dimension of the platform such as the bridge connecting two electrodes (source and drain electrodes) on which the tiny bioreceptor sensing molecules such as enzymes (e.g., DNA polymerase molecule), cells, antibodies, aptamers, peptides, or DNAs) are placed, with the sensing events comprising the singular or multiple attachment of analytes onto the bioreceptor molecule(s). As the analyte attachment event induces a quite small change in electrical conductivity of the base bridge platform, which is to be detected, it is important to maintain a reasonably low electrical conductivity in the bridge onto which the sensing bioreceptor biomolecule(s) are attached. The change in electrical conductivity in such sensors are often measured by, e.g., detecting changes in the amount of electrical current, voltage, electrical resistance (or impedance), or capacitance, for example, in the form of field effect transistors.

For the purpose of increasing the SNR in such bioreceptor-based molecular sensors, one of the useful approaches according to the disclosure is to make the bridge dimension small, for example, by (i) using a pre-made, small diameter bridge such as a molecular wire comprising DNA, RNA, peptide, or inorganic nanowire or nanoribbon, or (ii) utilizing a small dimension (thin and narrow) inorganic semiconductor bridge such as oxide semiconductors, graphene, metal dichalcogenide films like MoS₂ and other nanowire-shaped or nano-ribbon shaped materials. If the sensor bridge is made even thinner and/or narrower, the baseline electrical resistance (or impedance) will become higher, and hence the small electrical signal (or the change in electrical conductivity, measured by a change in electrical current, voltage or capacitance) induced by analyte attachment event onto the bioreceptor will not be buried in the low resistance electrical circuit.

Patterned semiconductor nanoribbons (such as ZnO, Al-doped ZnO, SnO, In₂O₃, GaN, ITO (indium tin oxide), IGZO (indium gallium zinc oxide,), doped Si or Ge etc.)) are fabricated by various methods. Some example techniques for thin film formation includes sputter deposition, evaporation, laser ablation, electrochemical deposition, chemical vapor deposition, atomic layer deposition (ALD), deposition of metallic layer followed by oxidation treatment, or spin coating deposition of salt precursor followed by oxidation anneal.

In certain embodiments, the semiconductor films may be directly deposited on a substrate or on device surface, or a precursor metal can be deposited first, followed by oxidation, sulfurization, caburization or nitriding. These semiconductors can be deposited and patterned directly on the nano-sensor electrode pair platform, or alternatively, they are pre-fabricated on a separate substrate, then transfer deposited on device electrode pair (source-drain) surface for nanobridge formation. The substrate for the semiconductor film deposition may include SiO₂/Si, sapphire, glass, ceramics, or for transfer deposit purpose, a metal or polymer substrate can also be utilized.

For reduction of nanobridge ribbon dimension, nano-fab patterning using photolithography a patterned mask array may be prepared using, for example, e-beam lithography, extreme UV (EUV) lithography, nanoimprint lithography, laser nanofabrication lithography, and so forth. For creation of nanopattern bridge to be dimension reduced, a polymer resist line array such as positive resist like PMMA or negative resist like HSQ, or other resist layer can be spin-coated, spray-coated, or dip-coated and patterned into nanobridge configuration first for further dimension reduction. The spacing between adjacent lines is made to be as narrow as possible, e.g., less than 40 nm, less than 20 nm or less than 10 nm, so as to obtain narrow nanoribbons.

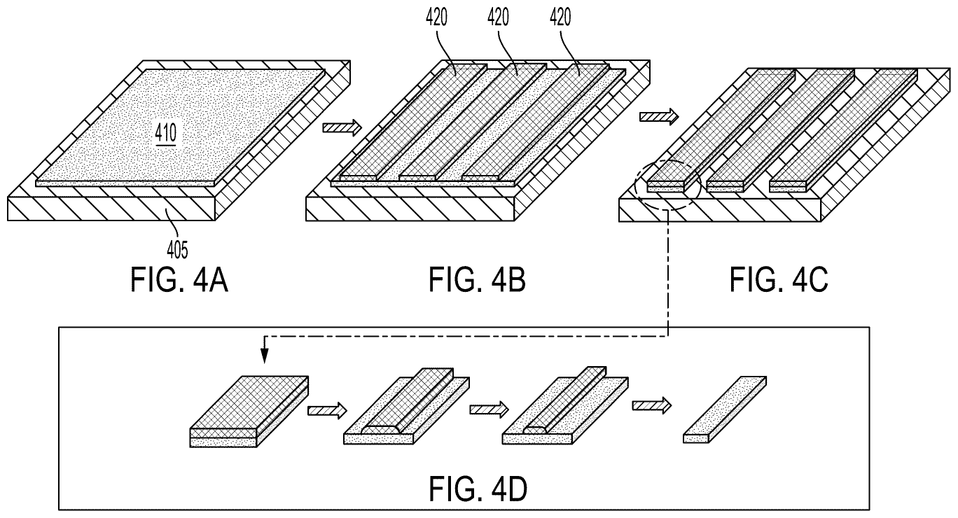

For additional nanoribbon width reduction according to the disclosed principles, an erodible mask line array or grid array is made first. Then the width of the resist line is reduced as illustrated in FIG. 4. In certain embodiments, the resist width reduction may be accomplished, by (i) RIE (reactive ion etch), (ii) chemical or electrochemical etch including acid etching, (iii) solvent etching, (iv) repeated (oxidation+etching) for gradual width reduction, (v) edge preferential oxidation, nitriding, sulfurization followed by edge preferential etching, or (vi) atomic layer etching or intentional e-beam damage of edge for more preferential etching away for width reduction. After the resist line width is reduced, the semiconductor layer underneath is nanoribbon fabricated using the RIE or chemical etching of the exposed semiconductors, thus leaving only the width-reduced semiconductor lines.

Such deposition of thin film, mask line deposition and width reduction can be performed either directly on device electrodes or on another flat substrate for subsequent transfer deposition onto device electrode pair array to form biosensor nanobridges. The desired amount of cross-sectional area reduction in the nanobridge by use of erodible mask line array for selected local etching erosion may be at least by 10%, at least 30% or by at least 50%.

FIG. 4(a)-(d) illustrates the use of erodible mask line array to create width-reduced and aligned semiconductor nanoribbon array on a flat substrate according to the disclosed principles. The semiconductor nanoribbon array may be deposited either directly on the sensor electrode pair or for subsequent transfer deposition onto device electrode pair array to form biosensor nanobridges. In step 4(a), semiconductor film 410 is formed over substrate 405. At FIG. 4(b), mask layers 420 are formed over semiconductor film 410. FIG. 4(c) illustrates the etching step in which the semiconductor layer underneath is also nanopatterned to relatively narrow ribbon arrays. FIG. 4(d) shows the operation for the gradual width erosion of the mask line according to the disclosed embodiments, for example, through RIE etch, ion etch or chemical etch. With the width-reduced mask lines, more desirable width-reduced semiconductor lines can be achieved upon etch patterning.

### Embodiment 4—Transfer Deposition of Ultrathin Oxide Type Semiconductor Film Ribbons, Microbridges or Nanobridges

While the metal-nonmetal semiconductor film can be in-situ deposited directly on the biosensor device surface and nanopatterned, there are occasions where the deposition is better carried out first on a separate substrate (in order to avoid damaging the device materials or circuits), and then either a large-area film or nanopatterned film is transfer deposited onto a device substrate. Examples of needs for separate thin film growth or processing (e.g., to further process the thin film materials for improved semiconductor properties) include; (i) Harsh thin film semiconductor growth conditions, which would damage/degrade the device base structures and properties, (ii) high-temperature treatments to anneal out undesirable defects, (iii) repair process to reduce damages previously induced by e.g., electron beam process, RIE ion etch process, (iv) to perform high temperature oxidation/reduction/nitriding/carburizing/phosphiding/sulfurizing type treatments, (v) to perform nanopatterning process which uses severe ion tech, severe acid or other chemical etch, and (vi) to add dopants by ion implantation.

High-temperature annealing heat treatment, e.g., at 50° C. to 500° C. for 1 min to 48 hours, can be performed optionally using a gas environment containing air, oxygen, nitrogen, nitrogen-containing gas, hydrogen, sulfur, phosphor, carbon-containing gas, and their mixtures, or vacuum environment to optimize the nanobridge semiconductor physical and electrical properties. Such a heat treatment process can be performed either before or after the nanofabrication into a nanoribbon configuration.

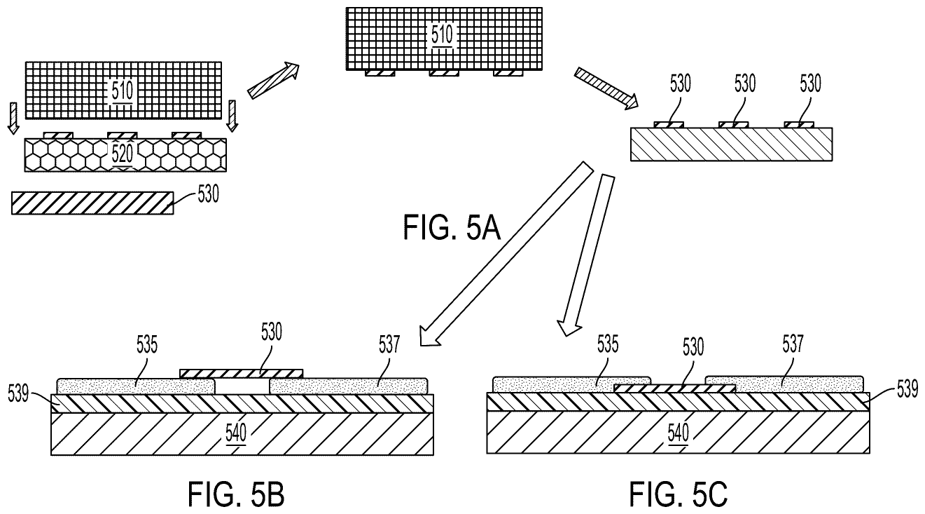

Shown in FIG. 5 is a schematic illustration of an exemplary transfer deposition of metal oxide type nanoribbons for electrode nanobridge formation. Specifically, in FIG. 5(a), a stamp transfer process via stamp 510 onto substrate 520 to form several semiconductor nano-ribbon arrays 530. In FIG. 5(b), nanoribbons 530 are transferred over metal electrodes 535, 537, which are formed over dielectric layer 539 and substrate 540. An alternative operation is illustrated at FIG. 5(c) in which nanobridge 530 array is positioned underneath the metal electrodes. In summary, the processes shown in FIG. 5 exemplify the transfer deposition of metal oxide type nanoribbons for electrode nanobridge formation in which FIG. 5(a) illustrates the stamp transfer process onto biosensor device, FIG. 5(b) illustrates the nanobridge array placed on top of the source-drain metallic electrode pair, and FIG. 5(c) illustrates nanobridge array 530 positioned underneath the metal electrodes.

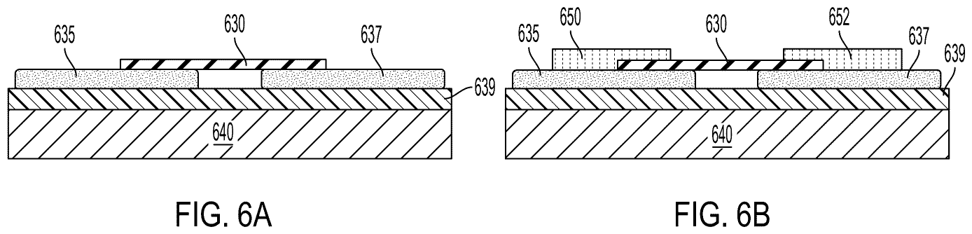

In FIG. 6, an adhesion stability of transfer-deposited metal oxide, metal nitride or metal carbide nanoribbon nanobridges on source and drain electrode surface is illustrated. Specifically, FIG. 6(a) illustrates the use of inherently strong van der Waals force of nanolayered materials for anchoring, optionally utilizing control of stamp pressure, time, temperature, humidity or a local pre-placement of a thin adhesive layer, so as to control and allow a stronger van der Waals bonding between nanobridge 630 and electrodes 635, 637 which are formed over dielectric layer 639 and substrate 640. In FIG. 6(b), an enhanced nanobridge adhesion is achieved by placing an anchoring structure 650, 652 on the outer edges of transferred nanobridge surface 630, e.g., by a transfer deposit of the anchor nanostructure or by a liquid polymer deposit (e.g., by nanoimprinting) and curing.

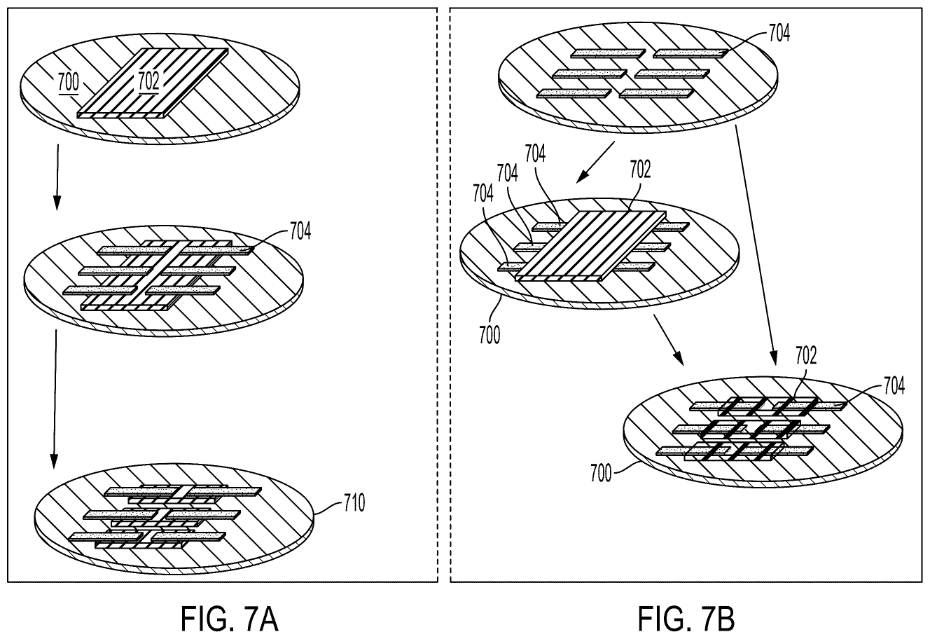

FIG. 7 is an exemplary operation illustrating a sequence of placement of the semiconducting oxide film (such as about 0.5-20 nm thick In₂O₃ film) relative to the metal electrode array placement. In FIG. 7, In₂O₃ film type semiconducting film 702 may be placed over substrate 700 first before the electrode 704 placement, followed by In₂O₃ film 704 patterning into a bridge configuration 710, FIG. 7(a), or the metal electrode array is formed first on the sensor device substrate, followed by In₂O₃ semiconductor film direct deposition or transfer deposit, and In₂O₃ film patterning. FIG. 7(b). The semiconductor film like In₂O₃ film can be directly deposited on the device substrate surface, e.g., by using RF sputtering, pulsed laser deposition (PLD), ALD, CVD, MOCVD, electrodeposition, or sol-gel type approach of spin coating or dip coating of thin layer aqueous salt-containing solution or dilute paste material followed by drying and baking heat treatment, e.g., 100-600° C., and possibly below about 350° C. to minimize any possible damage to the Si base or CMOS based circuit components.

In one embodiment of the disclosure, the In₂O₃ type semiconducting film can be grown or processed on another substrate (non-device substrate), then released (as a large-area film or patterned into macro, micro or nano ribbon array) from the substrate, and transfer deposited onto the sensor device substrate.

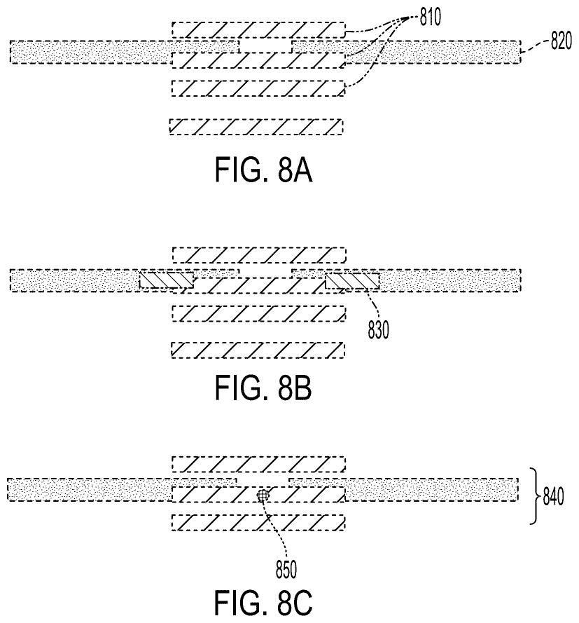

FIG. 8 is a top view of an array of metal electrode pairs with pairs with stamp-transferred inorganic nanoribbon bridges such as oxide, nitride or carbide type, with redundant ribbons to ensure at least one bridge formation occurs. In one embodiment, the array of array of metal electrode pairs (e.g., made of nanopatterned Au, Pt, Pd, Ru, Rh, or alloys) with PDMS stamp-transferred inorganic nanoribbon bridges such as oxide, nitride, carbide or other metal-nonmetal semiconductor compounds, with redundant ribbons (which might fall at the in-between locations next to the electrode locations) to ensure at least one bridge formation occurs.

In one application, the semiconductor nanoribbon array (oxide, nitride or carbide type) 810 is pre-aligned on another substrate in a parallel manner (width reduced as needed) and transfer deposited onto the electrode pair surface. The number and the density of parallel nanoribbons can be adjusted so as to provide a sufficient number of nanobridges on transfer deposition. In some embodiments, it may be acceptable to have redundant nanoribbons in the process of ensuring to have at least (or about) one nanobridge formed for each of the electrode pairs. For example, the spacing between adjacent nanoribbons is selected to be less than the width of the electrodes so that there is always at least one nanoribbon falling on electrode pair surface during the transfer deposit.

A further refined method of transfer deposit of semiconductor nanoribbons is to make the electrodes to vertically protrude, e.g., at least 10 nm, at least 20 nm, at least 50 nm, and at least 100 nm. With such a height difference, the elastomeric or adhesive stamp will contact only the electrode surface and not the empty recessed space between adjacent electrode pairs, thus transfer depositing the nanoribbons only on the protruding electrode surfaces.

The semiconductor nanobridge is in a thin ribbon configuration with a nanoscale dimension of less than 200 nm in width, less than 50 nm, less than 20 nm, less than 10 nm. The desired dimension of the oxide, nitride, carbide, or phosphide type semiconductor nanoribbons to be transferred has a desirable dimension of less than 200 nm in width, less than 50 nm, less than 20 nm, less than 10 nm. The desired thickness of the semiconductor nanobridge is less than 50 nm, less than 20 nm, less than 10 nm, less than 2 nm.

These semiconductor layers can be produced by thin film deposition such as sputtering, evaporation, pulsed laser deposition (PLD), ion deposition, atomic layer deposition (ALD), chemical vapor deposition, electrochemical deposition, precursor spin coating and baking, and so forth.

Optional deposition of anchoring metal, dielectric or polymer onto the outside ends of the transferred nanobridge nanoribbon can be performed using e.g., nanoimprinting, stamp transfer, or global deposition and patterning, for enhanced physical and electrical connection to the metal electrode surface.

Optionally, a masking coating with a molecular-adhesion-blocking agent may be added to prevent a linker molecule or enzyme molecule adhesion (e.g., biotin, streptavidin or polymerase), except for the local about 5 -25 nm circle, square or rectangle type space, or a line cavity on the nano-ribbon surface, so as to ensure only a single enzyme molecule (or a few if desired) is attached.

Array of protruding electrode or lead wires (Au, Pt, Pd, Ru, Rh or alloys thereof) for signal detection are depicted as 820 in FIG. 8(a)-8(c). The oxide, nitride or carbide type nanoribbons can be van der Waals force attached. Optionally, metallization deposit can be made to more firmly attach or anchor the nanoribbon bridge on the electrode. FIG. 8(b) shows the optional deposit of anchoring metal 830 which may be dielectric or polymer deposits. The anchoring metal 830 is placed over the outside ends of the transferred nanobridge nanoribbon to enhance the physical and electrical connection to the electrode surface. FIG. 8(c) shows one unit of FET sensor 840 comprising an inorganic (semiconducting) nanobridge with polymerase assembly. Optionally, two or three polymerase molecules can be used for each nanobridge. Enzyme polymerase (with DNA template) sensor 850 is attached onto nanobridge surface using molecular links.

### Embodiment 5. Amorphous Vs Crystalline Oxide Semiconductors for Biomolecular Sensors

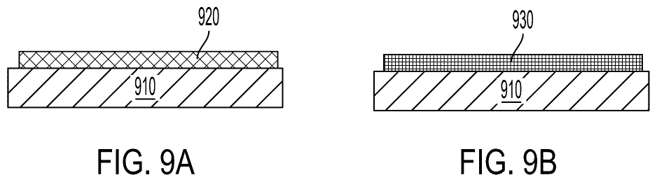

While crystalline semiconductor films have an advantage of being well-structured semiconductor materials, amorphous semiconductors (e.g., schematically shown in FIG. 9) have an advantage of avoiding grain boundary type defects which can cause undesirable noises and semiconductor properties. In order to produce amorphous (or almost amorphous) semiconductor films, the following exemplary and non-limiting techniques can be utilized according to the invention. generally lower temperature processing is preferred: (A) Use of low temp sputtering (substrate temperature Tₛ below −20° C., preferably below −100° C., more preferably at or below −196° C.); (B) Use of more complex oxide composition to make it more difficult to be crystalline film; (C) Use amorphous substrates or amorphous-material-coated substrates, or use a substrate with a large difference in lattice parameters as compared with that of the oxide semiconductor material; and (D) Use of ion implantation or neutron irradiation, etc. to disrupt the crystallinity and make it more toward amorphous structure.

In FIG. 9(A), the crystalline oxide semiconductor layer is deposited over the substrate, for example, by using an ambient temperature, raised substrate temperature or by post-annealing heat treatment. In FIG. 9(B) an amorphous oxide semiconductor layer is deposited over substrate, for example, by low temperature sputtering or by using ALD process.

### Embodiment 6—Insulating Gate FET Molecular Biosensor for Corrosion Protection of Ultrathin Oxide Semiconductor Nanoribbon and Unique Performances

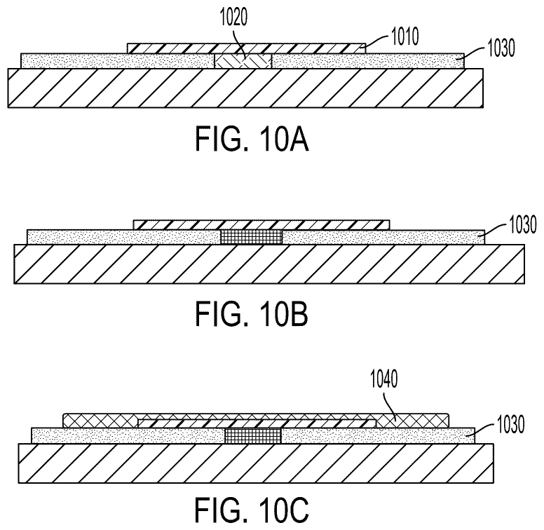

A variation of FET device is the use of a very thin insulating gate deposited on the semiconductor surface, above which a gate electrode structure is added as shown in the exemplary embodiment of FIG. 10. The biosensing molecules may be considered as another gate electrode. An important aspect for the biosensors used in a liquid environment (e.g., inside a microfluidic chamber, for genome sequencing or DNA memory, or a biosensor to be used by immersing inside a biofluid, such as a blood drop, sweat, urine, or saliva samples) is the need for corrosion protection for longevity of the semiconductor bridge. The corrosion protection material may include indium oxide based (including In₂O₃, ITO, IGZO, ZnO based, SnO₂ based), CuO based, NiO based semiconductor and may be used on the bridges which are placed inside an aqueous solution. A dual purpose anti-corrosion and insulator-gating, thin dielectric cover (e.g., SiO₂, Si₃N₄, or ALD-deposited Al₂O₃, ZrO₂, HfO₂) can either directly be deposited to cover the nanobridge or stamp transfer deposited.

FIG. 10(a) shows a dry ultrathin oxide semiconductor nanobridge 1010 which may comprise In₂O₃, In₂O₃, ITO (indium-tin-oxide), IGZO, ZnO, SnO₂, etc. The nanobridge 1010 may be suspended or it may be placed on a planarized surfaces 1020 (e.g., with the gap between the source and the drain electrodes filled by silica) and electrodes 1030. FIG. 10(b) shows the embodiment of FIG. 10(a) in a biosensing liquid solution. The biosensor may be susceptible to corrosion in a wet environment especially when the liquid environment contains salts ions and an applied electric filed. FIG. 10(c) schematically illustrates a dual purpose, anti-corrosion and insulator, thin dielectric cover 1040. Insulator film 1040 may comprise SiO₂, Si₃N₄, or ALD deposited Al₂O3, HfO₂, either directly deposited or stamp transfer deposited on nanobridge 1010.

The liquid environment frequent encountered in biosensor assays or disease molecule sensing is generally supplied into a microfluidic chamber, which is often made of aqueous solutions containing, e.g., phosphate buffered solution (PBS) or salt solution for supply of analyte molecules or proteins (virus, bacteria, A, C, G, T type nucleotides for genome sequencing, or other chemical analytes). Thin oxide semiconductor nanobridge materials such as indium oxide are sometimes susceptible to corrosion in aqueous environment especially with commonly encountered some salt ions and applied electric field.

Dual purpose anti-corrosion and insulator-gating dielectric cover 1040 (e.g., sputtered or CVD deposited SiO₂, Si₃N₄, Al₂O₃, refractory metal oxide, rare-earth oxide, or ALD-deposited tight Al₂O₃), may be either directly deposited to cover the nanobridge or stamp transfer deposited. The thin oxide semiconductors are protected from the biological aqueous solution by the dielectric layer on top. Other materials such as mixed oxides, nitrides, fluorides, oxynitride, oxyfluoride, etc. can also be utilized. Instead of ceramic dielectric, a polymeric dielectric layer can also be utilized for the insulating gate arrangement.

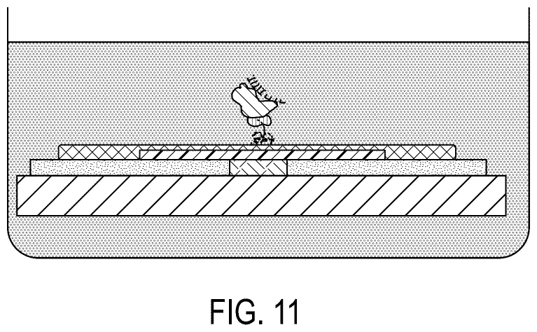

FIG. 11 schematically illustrates an insulator-gated FET molecular biosensor, with a very thin insulating gate arrangement. Different configurations such as a two electrode, no-added-gate FET molecular biosensor, FIG. 10(b), or an insulator-gated FET molecular biosensor with a thin insulating layer, FIG. 10(c) may be utilized in the embodiment of FIG. 11. A single enzyme molecule with a DNA template can serves as a modulating gate on nucleotide attachment. The insulator gate material can be selected from, e.g., Si₃N₄, SiO₂, Al₂O₃, refractory metal oxide (e.g., HfO₂ or ZrO₂), rare-earth oxide, or their combinations.

In certain embodiments, the desired thickness of the insulator gating layer which also protects the oxide semiconductor nanoribbon from corrosion, may be in the range of 0.2-50 nm, 0.5-10 nm or 0.5-5 nm. In addition to the FET structure comprising oxide semiconductor ribbons, the device can also optionally comprise a bottom gate, e.g., using the SiO₂/Si substrate.

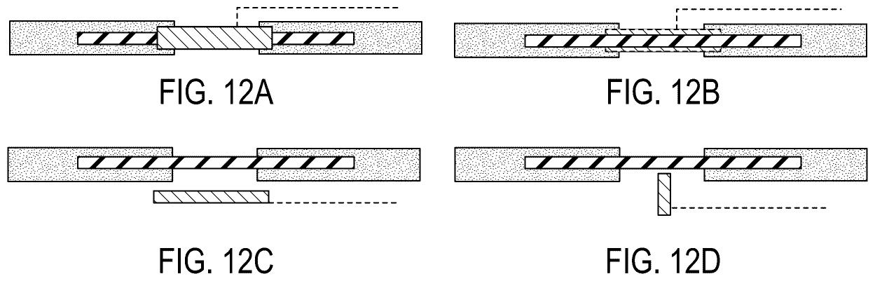

FIG. 12 shows top view schematics of various conductor gated structures of oxide-semiconductor-nanobridge based molecular biosensors. Specifically, FIG. 12 shows various conductor gated structures of nanobridge-based molecular biosensors. The top gate structure comprises the polymerase molecular sensor molecule directly attached on top of the In₂O₃ nanobridge, with the conductive gate placed above the nanobridge with a gap spacing of e.g., 50 nm-5 um. The conductive gate (e.g., Si or other metallic back gate) can also be positioned on the bottom region of the electrode pair, side in a parallel orientation or perpendicular orientation, or on the side of the electrode pair in a parallel orientation or perpendicular orientation.

The gate arrangement and configurations such as the gate location and gate orientation angle can be altered for convenience and for various purposes, as illustrated in FIG. 12(a) to 12(c). Top view of various conductor gated structures of e.g., oxide-semiconductor-nanobridge based molecular biosensors are shown, with the top gate configuration comprises the biosensor molecule (e.g., DNA polymerase enzyme molecule sensors, aptamer sensors, peptide sensors, and so forth) attached on top of the nanobridge (FIG. 12(A)). One or more gates can also be positioned on the bottom region of the electrode pair (FIG. 12(B)), side in a parallel orientation (FIG. 12(C)) or perpendicular orientation (FIG. 12(D)), on the side of the electrode pair in a parallel, perpendicular or angled orientation. The back gate or side gate may be made of Si or metallic layer material.

### Embodiment 7—Semiconductor Thin Film Release from Growth Substrate and Transfer Deposit with the Film Separation Optionally Aided by Sacrificial Interface Layer

While the semiconductor film and be directly deposited on the device surface and nanopatterned, this often imposes some restrictions in materials processing, e.g., in terms of exposure temperature, time, gas atmosphere, chemical or ion etch steps, and so forth, as might be required to safeguard the device structure. Therefore, it is sometimes convenient to perform the film preparation and nanobridge patterning process on a separate substrate, without processing restrictions, and then transfer deposit the pre-patterned nanobridge, or preferably a nanobridge array, to the device surface. For transfer deposit of semiconductor thin film (or patterned micro ribbons or nanoribbons), the film needs to be separated from the growth substrate. For such transfer deposit, elastomeric stamps, adhesive stamps, or thermally releasable polymeric adhesive layer material can be utilized to pick up the film and release it onto the final device substrate (for biosensor construction), or to another substrate for additional required processing such as high temperature annealing, oxidation, nitriding, carburizing, or phosphating treatment, ion implantation treatment, etc. These approaches are summarized in the figure below.

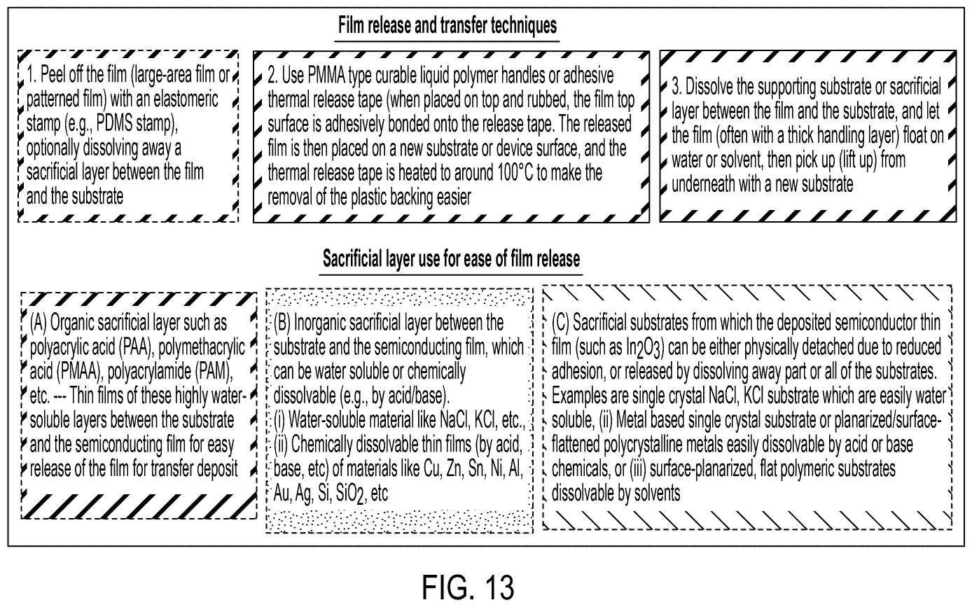

Semiconductor thin film releasing/detaching for biosensor applications (e.g., In₂O₃ film of e.g., 0.5-20 nm thick) from a growth substrate is useful and is sometimes essential for unique sensor assembly. Shown in FIG. 13 are some process descriptions for semiconductor thin film releasing/detaching for biosensor applications (for example, In₂O₃ film of e.g., 0.5-20 nm thick) from a growth substrate. The transfer of thin film, either as a large-area film or as a prepatterned into nanobridge array, is useful for FET type biosensor construction. The semiconductor film, either as (i) as-grown film on a substrate, (ii) a processed film (e.g., annealed, oxidized, nitrided, carburized, sulfurized, phosphorized, ion-implanted, etc), (iii) previously transferred film, or (iv) lifted-up film (from floating status in a liquid), needs to be released first from the substrate in order to allow a transfer onto a device or onto another substrate.

Organic sacrificial layers such as polyacrylic acid (PAA), polymethacrylic acid (PMAA), polyacrylamide (PAM), etc. are highly water-soluble. A thin layer can easily be deposited on the substrate by spin coating, dip coating, spray coating, sputtering, etc., in a desirable thickness regime of e.g., about 5 nm to 10 um. A desired thin film semiconductor can then be deposited on top of these sacrificial films, after which the sacrificial film can be dissolved away to release the film in water or other solvents (or burned away by heating). The floating film can then be collected by lifting up with a carrier substrate or device substrate from underneath. A thicker handling support such as polymer or metal can optionally be prepared for ease of handling a very thin and fragile film.

Inorganic sacrificial layer, which can be water soluble or chemically dissolvable (e.g., by acid/base) include: (i) Water-soluble materials like NaCl, KCI, etc. which can be deposited on the substrate surface before deposition of semiconductor thin films. Thin films of these highly water-soluble inorganic sacrificial layers can be deposited on the substrate by sputtering, sublimation, pulsed laser deposition or aqueous solution spin coating (optionally with a removable binder material); (ii) Chemically dissolvable thin films of materials like Cu, Zn, Sn, Ni, Al, Au, Ag, Si, SiO₂ and so forth, in a desirable thickness regime of e.g., 1-200 nm, preferably 2-20 nm can be utilized. A desired thin film semiconductor can then be deposited on top of these sacrificial films, after which the sacrificial film can be dissolved away to release the film. The floating film can then be collected by lifting up with a carrier substrate or device substrate from underneath. As the released film is very thin and fragile film, a thicker handling support such as polymer or metal can optionally deposited on top of the semiconductor film for ease of handling.

Sacrificial substrates can be utilized for semiconductor film deposition, from which the deposited thin film semiconductor (such as In₂O₃) can be either physically detached due to reduced adhesion or released by dissolving away the sacrificial substrate. Examples of dissolvable substrates include, (i) Single crystal NaCl, KCI sheets/plates which are easily water soluble, (ii) Metal based single crystal substrate or planarized/surface-flattened polycrystalline metals such as Cu, Zn, Sn, Ni, Al, Au, Ag, Si, SiO₂, and so forth, which can be easily dissolved away by acid or base chemical solutions, or (iii) Surface-planarized, flat polymeric substrates such as PMMA, SU-8, HSQ type resist materials which can be dissolved away by solvents or burned away by heating.

### Embodiment 8—Nanobridge Width Adjustments to Control the Number of Attached Biosensor Molecules

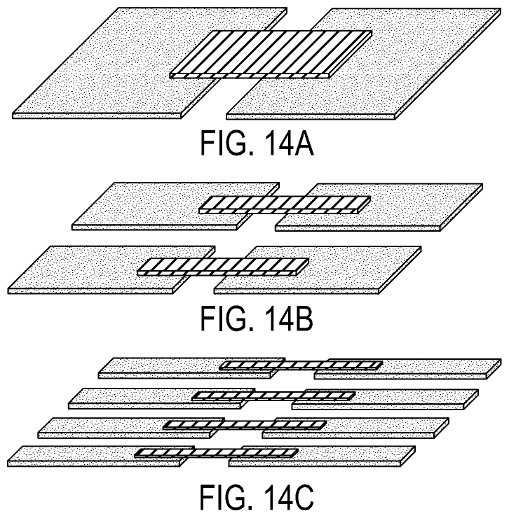

A substantially narrower-bridge structured biosensor, for example illustrated at FIG. 14, enables (i) a higher SNR of the biosensor molecule on the nanobridged field-effect-transistor (FET) devices (e.g., from the sensor molecules of DNA polymerase enzyme sensor, aptamer sensor, DNA sensor, antibody sensor, glucose sensor, stress hormone sensor, or other protein sensors, and so forth), (ii) a narrower ribbon bridge restricts the number of sensor biomolecules to be attached on the bridge surface, which limits interference from neighboring sensor biomolecules, especially when genome or DNA sequencing is performed, and (iii) the narrower bridges allow a higher density of individual sensor parts on a given-area biosensor, which enables more parallel analysis of analytes.

In one embodiment, the width of the oxide semiconductor nanoribbon (e.g., In₂O₃ type) is in the range of about 2-500 nm, 5-100 nm or 5-20 nm. The desired thickness of the oxide semiconductor nanoribbon may be in the range of about 0.5-20 nm, 1-10 nm or 1-5 nm. The desired length of the oxide semiconductor nanoribbon may be in in the range of 20 nm-50 um, 100 nm-10 um or 200 nm-1 um.

However, a wider bridge of for example, 500 nm to 100 um width, is not excluded in the disclosed principles as such a macro width bridge can contain many biosensor molecules to provide overall large sensor signals for binding detection type applications for sensing of disease-causing antigens/molecules or detection of physiological cues for health monitoring type applications. On such exemplary embodiment is represented and discussed in relation with FIG. 14.

FIG. 14(a) to 14(c) illustrate nano or micro bridge width control of In₂O₃ type semiconducting film. FIG. 14(a) schematically illustrates an exemplary wider bridge array. FIG. 14(b) illustrates an intermediate width bridge array. FIG. 14(c) illustrates nanoscale narrower bridge array. An array of narrower bridge such as nano-dimension-width wide bridges of less than 50 nm can provide various advantageous properties for the biosensor as discussed above.

### Embodiment 9—Restriction of Biomolecule Binding Sites Via a Pre-Patterned Mask to Control the Location and Number of Attached Biosensor Molecules

A masking layer that blocks the attachment of biosensor molecules onto the semiconducting bridge surface can be prepared with a proper nanopatterning with only a specific, limited area to be open, for example as a hole array or a slot array mask pattern. These biomolecule-blocking dielectric mask can be made of a polymeric material or a ceramic material and can be prepared in-situ directly on the finished biosensor device, followed by sensor biomolecule attachment through the exposed openings. Alternatively, such masks can be deposited and patterned on a separate substrate, released, and then transfer deposited on the device nanobridge architecture to enable the desired restriction of sensor molecule biding sites.

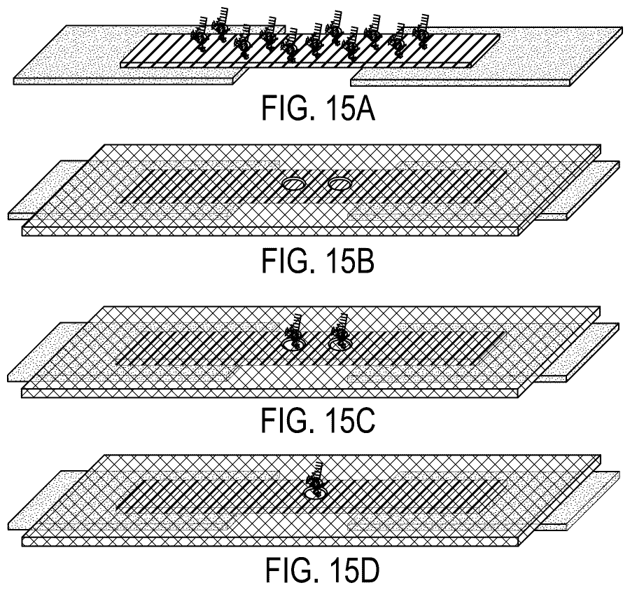

FIG. 15 schematically illustrates an exemplary embodiment for controlling the number of bioreceptor sites for attaching the sensor molecule (e.g., aptamers or polymerase enzymes) to the semiconductor microbridge or nanobridge surface. The exemplary embodiment of FIG. 15 is useful for, among others, DNA sequencing, detecting disease-causing antigen or detecting the antibody levels.

As illustrated in FIG. 15, the number of bioreceptors (sensor molecules such as aptamers, polymerase enzyme molecules, DNAs, peptides) attached onto the semiconductor microbridge (e.g., In₂O₃) or nanobridge surface can be controlled by using a mask structure. In FIG. 15(a), many bioreceptor sensor molecules, for example, at least 3, at least 50, at least 200 are allowed to couple to the bridge. Such a bulk type biosensor is useful for a binding assay type detection of disease-causing antigens, metabolic status and nutrient analysis from a biofluid, e.g., in a clinic type environment in which microfluidic chamber can be utilized for analysis of patients' blood, saliva, urine, and other body fluids.

Such a binding assay type detection can also be utilized as a miniature wearable biosensor for sweat analysis to provide key biofunction parameter information such as electrolytes, nutrients, metabolic parameters, pH level, and stress hormone cortisol analysis or other physiological parameter analysis. Other biofluids such as blood, urine, saliva, nose fluid, etc. can also be analyzed for health care purposes like a desired analysis of nutrients, glucose, hormone, antibody, antigen, or metabolic status.

In order to control the attachment of bioreceptor molecules, the nanobridge in this invention can be covered by a dielectric mask that blocks the adhesion of bioreceptor biomolecules, but with one or more openings which allow the attachment of bioreceptor molecules only in the opening area, so as to restrict the number of bioreceptor molecules attached on its surface.

FIG. 15(b) schematically illustrates an exemplary structure of the biomolecule-blocking dielectric mask coating, which can be selected from polymeric materials, ceramic materials or composite materials. Polymeric mask materials that tend to prevent biomolecule attachment can be, for example, polymethyl methacrylate (PMMA), SU-8, polytetrafluoroethylene (PTFE), polyethylene glycol (PEG), polydimethylsiloxane (PDMS), or related elastomers, polystyrene (PS), polyurethane (PU), or other thermoplastic or thermosetting materials. Ceramic type mask materials can be selected from e.g., SiO₂, Al₂O₃, MgO, TiO₂, ZrO₂, Ta₂O₅ or other refractory oxide layer. The polymeric or ceramic mask coatings can be applied by spin coating and nanopatterning or physical or chemical vapor deposition such as sputtering, evaporation, atomic layer deposition (ALD), followed by lithographic patterning such as e-beam lithography, photolithography, laser lithography, or nanoimprint lithography.

For ease of nanopatterning as well as to minimize interference of bioreceptor molecule attachment within the small opening, the wall thickness of the mask layer is desirably thin. In some embodiments, the thickness of the mask layer may be less than 200 nm, less than 100 nm, less than 20 nm or less than 5 nm.

In order to have a controlled and quantifiable bioreceptor electrical responses, the nanobridge may comprise a restricted number of openings bioreceptor molecules attached on its surface, for example, less than 20, less than 5 or only one, with the latter desirable for accurate electronic biosensing, especially for genome sequencing type applications. The nanoscale opening in the mask layer can be circular, square, rectangular or irregular shape on each nanobridge, or a slit-type opening spanning multiple nanobridges, for selective bioreceptor molecule attachment. The opening in the mask for bioreceptor molecule attachment desirably has an average diameter (or equivalent diameter covering the same area) of less than 500 nm, less than 50 nm, less than 10 nm or less than 6 nm.

Instead of circular or square openings, a long slot opening shape can also be utilized for an array of semiconductor nanobridge, so that each long slot opening provides an opening for all the bridges in that row or column. The slit-type opening for bioreceptor molecule attachment is desirably made to span many nanobridges, e.g., more than 10 nanobridges, preferably more than 100 nanobridges for ease of fabrication. The desired width of opening for the slit is less than 500 nm, preferably less than 50 nm, more preferably less than 10 nm, even more preferably less than 6 nm.

The hole-array-patterned or slot-array-containing dielectric mask layer can be in-situ deposited and patterned, or liquid precursor dispensed by nanoimprinting or nano ink-jet-type dispensing and cured during the device construction, or alternatively it can be prepared separately on another substrate, released/separated from the substrate, and then transfer deposited on top of the nano/micro bridge array.

In FIG. 15(c), two biosensor molecules (i.e., two identical or two different probe biomolecules of e.g., a DNA polymerase and an aptamer, or two different types of aptamer molecules) are attached on the nanobridge because only two small-area mask openings are made available. In order to construct a micro/nano bridge comprising a multiple but different types of probe molecules (e.g., a combination of different types of DNA polymerase, different DNA probes, different aptamers), each bridge surface in a bridge array sensor assembly can be pre-treated or pre-coated with one or more functionalities to enhance or to selectively allow attachment of specific probe molecule types on the desire locations.

Such pre-coating includes either a component or a combined attachment-functionality-complexes of, e.g., silane type layer or complexes, or a pre-coating with thiol complexes, biotin-streptavidin complex, antigen-antibody complex, pyrene-maleimide complexes, other connecting moiety, e.g., azide- or amine-related click chemistry, tag-binding related links, and so forth. These pre-coatings to enhance probe-molecule-attachment can optionally be applied before the hole containing probe-inhibiting mask is placed on top. This can be accomplished, e.g., by stamp transfer placement of the mask, or by using a scanning nano-pipette array that releases various types of precursor mask layer like curable liquid dielectric material such as PMMA, SU-8, etc. at desired locations with a desired nanopattern. Alternatively, the functional complexes could be applied after the probe-inhibiting mask is placed first.

In FIG. 15(d), only a single opening is provided in the mask to allow a single-molecule biosensor on each nanobridge, which is useful for DNA or genome sequencing, in which unambiguous electrical signal is desired for each nucleotide attachment event. In the biomolecule probe sensor array comprising an array of microbridges or nanobridges, the biosensor device can advantageously be a single molecule biosensor per semiconductor bridge. For redundancy and other quality control purposes, as well as for consideration of probe molecule attachment efficiency or for multi probe array, a two-molecule biosensor per bridge, a three-molecule biosensor per bridge, or up to ten-molecule biosensor per bridge can be utilized as desired for binding assay type sensing.

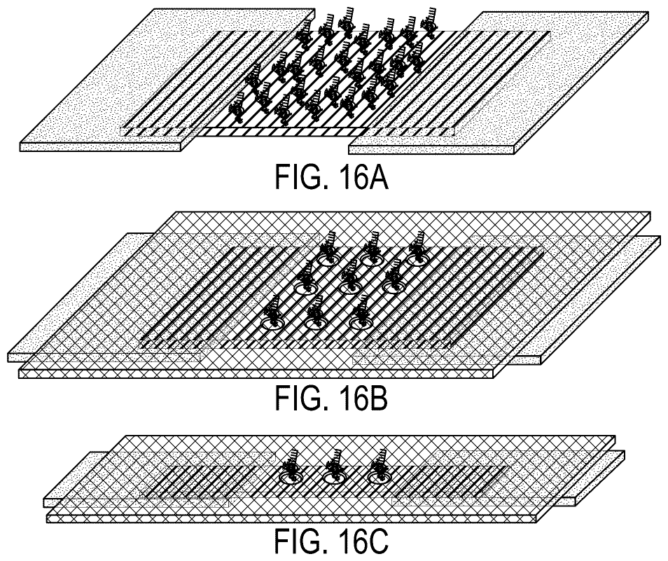

Schematically FIG. 16 schematically illustrates a semiconductor bridge array physically secured by metal electrode array on top. Specifically, FIG. 16(a) shows multiple bioreceptors coupled to the nanobridge surface. FIG. 16(b) schematically illustrates several circular open holes patterned on the dielectric ceramic or polymer mask. Only the exposed areas allow the bioreceptor (sensor) molecules to attach to the semiconductor. FIG. 16(c) shows only three sensor molecules attached per each bridge onto the unmasked areas of the nanobridge.

The sensor molecule position- and number-restricting open holes (or squares, rectangles, and so forth) can have an equivalent diameter of less than about 500 nm, less than 50 nm, less than 10 nm or less than 6 nm. The thickness of the mask layer can be less than 100 nm, less than 50 nm, less than 20 nm or less than 10 nm. The patterning can be done by lithography such as using e-beam lithography, EUV lithography, interference laser lithography, nanoimprinting, and so forth). The dielectric ceramic mask can be selected from the group consisting of SiO₂, Al₂O₃, TiO₂, ZrO₂, HfO₂, or polymer mask such as PMMA, SU-8, or other thermoplastic or thermosetting material. Only the exposed areas allow the bioreceptor (sensor) molecules to attach, couple or connect to the semiconductor. In FIG. 16(c) only three sensor molecules (e.g., aptamer of polymerase enzyme) are allowed to attach to the bridge at the unmasked areas of the semiconductor bridge (channel) surface. The mask may be either directly processed on top of the device or it may be prepared on a separate substrate and transfer deposited.

The number of bioreceptor attachable sites is precisely controlled on the In₂O₃ type or other semiconductor microbridge or nanobridge surface by a pre-determined number of open holes in the superimposed mask structure. Thus, in the sensor array, the sensor-to-sensor uniformity in signal generation is more uniform as compared to the case of random, uncontrolled, and varying number of bioreceptor molecules getting attached on different sensor bridges. Agglomerated sensor molecule deposition, sometimes occurring in a random attachment route, is minimized with the precise position-and number-restricted attachment.

Such biosensors with many sensor molecules are useful for medical applications, for example, in a binding assay type analysis for disease-causing antigen, antibody levels, nutrient levels, stress hormone cortisol detection, electrolytes, pH levels, and various other physiological parameters, e.g., from wearable biosensors or clinic type analysis. Such a biosensor with many sensing molecules is useful for analysis of various types of biofluids such as blood, urine, saliva, nose fluid, etc., which can also be utilized or health care purposes.

In the case of multi-biomolecule probe sensor, the biomolecule attachment sites can be a random or a periodic arrangement on the nanobridge. The spacing between neighboring sites is controlled and kept small to achieve a high density of probe molecules. In one embodiment, the spacing between neighboring bioreceptor biomolecule sites may be less than about 10 um, less than 1 um, less than 100 nm or less than 50 nm. The density of biomolecule probes may be at least 10⁶/cm², at least 10⁸/cm², at least 10¹⁰/cm² or at least 4×10¹¹/cm².

The exact position and number of the attached biosensor molecules may be empirically obtained to verify the presence of intended number of biomolecules and to perform quality-control of the biomolecule position, the number and the distributions, e.g., by using wet AFM analysis or dry SEM analysis using pre-dried samples.

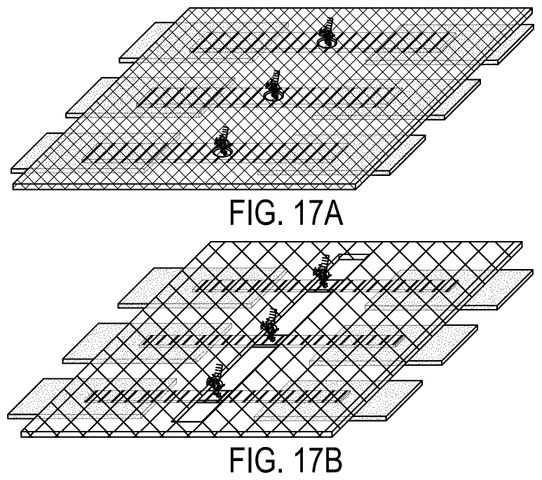

Shown in FIG. 17 are some example shapes of the biomolecule-blocking dielectric mask which can contain discrete circular/square/rectangular/oval shaped hole openings for selective attachment of biosensor molecules (and hence selected number of the sensor molecules) on the semiconductor bridge. The semiconductor nanoribbons are placed below the metal electrodes which physically and electrically secures the nanoribbons. The supporting substrate, for example, 300 nm SiO₂/Si is not shown in FIG. 17. In FIG. 17(a), circular openings in the mask are shown. In FIG. 17(b), alternative to the discrete hole openings in FIG. 17(a), an elongated slot shape opening in the mask is illustrated which more conveniently allows specific sites in all bridges in a nanobridge array to be available for biosensor molecule attachments. The number of available attachment sites in a multi bridge configuration is also specified.

### Embodiment 10—Biosensor Assembly for Various Biotech Applications

The biosensor structures described in this invention can be useful for various analysis of analytes or disease-causing micro-organisms, as well as detection of proteins and chemicals for nutrient analysis, hormone analysis, metabolic parameters, and so forth. Some basic structures and assembled biosensors are schematically illustrated in the following figures. (The microfluidic chamber and supporting frames, electronics and computing devices are not shown).

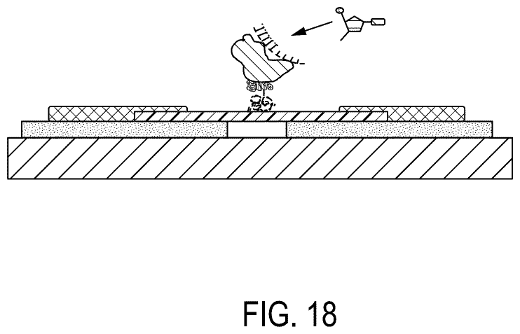

FIG. 18 schematically illustrate a DNA or genome sequencing application of ultrathin oxide FET molecular biosensor in a microfluidic chamber (not shown), detecting the nucleotide attachment by electronic signals according to one embodiment of the disclosure. The location-dictating-mask for selected-site attachment of polymerase type sensor molecule is not shown. Specifically, the drawing schematically illustrates a biomolecular biosensor based on oxide type semiconductor FET for DNA analysis such as a detection of nucleotides getting attached onto DNA template associated with one or more bioreceptor sensor molecule, e.g., for genome sequencing via electronic signals.

The oxide nanobridge may be suspended over a pair of electrodes (source and drain electrodes) or placed on planarized surface of electrode assembly. The nanobridge may also be organic type like DNA or peptide. The electrodes can be selected from conductive metals or alloys, preferably relatively inert metals or their alloys including Au, Pt, Pd, Ru, Rh or alloys. The placed nanobridge(s) tends to attach onto the metal surface by strong van der Waals force. However, to ensure good electrical and physical connections, an optional anchoring dielectric (or metal) may be added near the far ends of the nanobridge.

Bioreceptor sensor molecule (such as one or more enzymes, DNA Taq polymerase type single enzyme molecule, cells, aptamers, peptides, DNAs) may attach to the nanobridge surface according to certain disclosed embodiments. In some embodiments, the polymerase sensor molecule may include a DNA template with one or more primer components. The bioreceptor may be connected to the nanobridge platform, for example, by using biotin-streptavidin complex antigen-antibody, pyrene-maleimide complexes (with other connecting moiety, e.g., azide- or amine-related click chemistry, tag-binding related links, silane-related links, and so forth. When the analytes such as nucleotide monomer (e.g., A, T, C, G) are attached to the DNA template, the change in electrical sensor signal is detected so as to allow genome or DNA sequencing analysis. The analytes can also be disease-inducing virus or bacteria such as COVID virus.

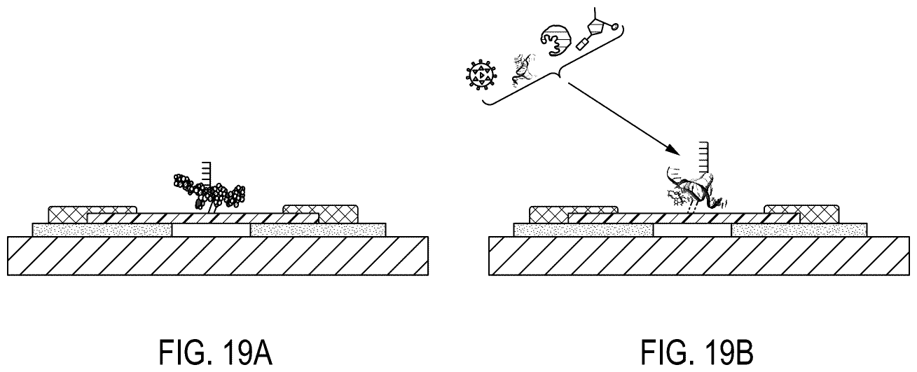

FIG. 19 schematically illustrates FET molecular biosensors in a microfluidic chamber environment (not shown). The FET sensor may comprise oxide semiconductor nanobridges for detecting various analytes including COVID-19 virus, other coronavirus types, disease-inducing or epidemic-causing virus or other microorganisms, proteins or DNAs in forensic analysis, sensing of environmental pollutants (organic or inorganic) or DNA analysis. FIG. 19(a) shows peptide based sensor molecules with primers and FIG. 19(b) shows aptamer based primer-added sensor molecules. The sensor molecule location-dictating-mask is not shown in FIG. 19.

As illustrated in FIG. 19 one or more bioreceptor sensor molecules, such as peptides with primers added or aptamers with a primer attached) may be coupled to a high SNR nanobridge surface. The nanobridge may be width-reduced and thickness-reduced. Bioreceptor connection to the nanobridge platform can be accomplished using connective functionalization (conjugating moieties), e.g., using biotin-streptavidin complex antigen-antibody, pyrene-maleimide complexes (with other connecting moiety, e.g., azide-or amine-related click chemistry, tag-binding related links, silane-related linkers, thiol-related linkers, PEG (polyethylene glycol) related linkers.)

In certain embodiments, the analytes to be detected may include one or more disease-inducing or epidemic-causing virus (coronavirus including COVID-19 virus, SARS or influenza virus, hepatitis, HIV, hepatovirus), or other micro bacteria and organisms, nucleotides in DNA analysis, proteins or DNAs in forensic analysis, enzymes, hormones, albumin, peptides for health examinations, sensing of environmental pollutants (organic or inorganic) including volatile organic compounds. Electrical signals may be used to detect a change in voltage, current, impedance or capacitance upon the analyte molecule attachment event.

### Embodiment 11—Nanoscale Biosensors for Wearable Sweat Analysis for Personal Healthcare

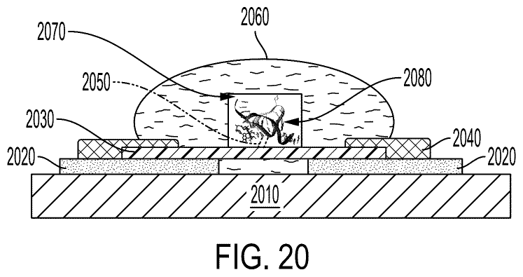

In one embodiment, the disclosure relates to a wearable biosensor which may be used to detect body analytes for personal healthcare. The nanoscale biosensors disclosed in this invention may be miniaturized and used to detect various biological and electronic applications by providing substantially higher density while being more compact and lighter than the conventional detectors. A wearable sweat sensor may be produced using such miniaturized device comprising a nano sensor array, as illustrated in FIG. 20. The nanoscale biosensors may also be prepared on flexible substrates for advantageous wearable configurations. In certain applications, the nanoscale biosensors may be significantly miniaturized such that a detection chip and circuitry may be mounted on an overall flexible support structure like plastic and/or polymer layers. In this manner, the device can provide flexibility.

Wearable sensor technologies can help to enable personalized healthcare, an important trend toward precise medicine approach, which presents significant potential in the level of billion dollar market. The nanoscale biosensors can provide more powerful and more wearable devices compared to the current state-of-the-art microscale or macroscale biosensors.

Therefore, in addition to genome sequencing, drug delivery, disease detection such as COVID, and DNA memory type applications described in the specification, such nanoscale biosensors devices comprising sensor-molecule-site-dictated bioreceptor molecules and dimension-controlled semiconductor nanobridges are also useful for wearable bio sensors such as sweat sensor for detection/collection of physiological information from human sweat and other human biofluids, including quantitative analysis of electrolyte and pH levels, nutrient levels, metabolic status, stress hormones (such as cortisol), and so forth. Wearable sensor technologies can help to enable personalized healthcare, an important trend toward precise medicine approach, which presents a huge potential in the level of multibillion dollar market. The nanoscale biosensors can provide more powerful and more wearable devices compared to the current state-of-the-art microscale or macroscale biosensors.

As illustrated in FIG. 20, the wearable (or instant-analysis) FET molecular biosensor can be utilized for biofluid 2060 analysis in a non-microfluidic chamber environment (e.g., from sweat or blood droplet, or various other human biofluids like saliva, nasal mucus, urine, and so forth. Such oxide semiconductor nanobridge sensors can also be used for detection of various other analytes including metabolic status, hormone levels, and so forth.

In FIG. 20, one or more bioreceptor sensor molecules 2080, such as peptides with primers added or aptamers with a primer attached, can be placed onto a high SNR nanobridge surface 2030, optionally width-reduced and thickness-reduced. The bioreceptor connection 2050 to the nanobridge platform may comprise a biotin-streptavidin complex antigen-antibody, pyrene-maleimide complexes (with other connecting moiety, e.g., azide- or amine-related click chemistry, tag-binding related links, silane, and so forth.) Primers 2070 for binding attachment to the analytes may also be used. FIG. 20 schematically represents aptamer type, one or more bioreceptor sensor molecules 2080 (for example, with primers added) and placed onto the high SNR environment 2060. Nanobridge 2030 may be anchored by 2040 end-anchoring coating over electrodes 2020 and substrate 2010 (e.g., Si substrate with a 100-500 nm thick thermal oxide SiO₂ dielectric surface). Thus, analytes such as virus, COVID variants, bacteria, related organisms, proteins, enzymes, genes, albumin, inorganic/organic environmental pollutant, and so forth in the biofluid 2060 can be detected.

Metabolic analysis (electrolytes, pH levels, nutrient levels), hormones (such as stress hormone cortisol, insulin, and so forth) can also be detected in the wearable sensor format or as an instant analysis device. Electrical signals for biosensing utilizes measurements of changes in voltage, current, impedance or capacitance upon analyte molecule attachment event.

### Embodiment 12—Biosensor Assembly Comprising Thin Metal-Nonmetal Compound Semiconductor Bridge Array for DNA Memory Applications

DNA data storage is a process of digital encoding and decoding binary data, to and from synthesized or duplicated DNA strands. For example, the binary code information storage of (00), (01), (10) and (11) can be replaced by various arrangements of oligonucleotides (A, C, G, T). As is well known, DNA molecules are genetic blueprints for living organisms, and the information stored in DNA is known to last more than 10,000 years under certain environment. With its huge capacity (many orders of magnitude larger than what is possible with current technology) to store enormous amount of information in very small space, DNA storage could be the answer to a modern era problem of too much information that needs to be stored, e.g., on the order of hundreds of zettabytes every year in the near future. Currently available information storage capability including magnetic disk, tape, optical or other related technologies can cover only a fraction of such a needed capacity.

While substantial progress has been made in DNA information storage in recent years, cost effective data storage techniques for practical applications are yet to be achieved. For efficient retrieval of stored information, the encoded DNA nucleotide arrangements need to be decoded by, e.g., reading (or sequence analysis). A fast, economic method of reading the encoded DNA information is essential for the success of DNA based data storage. This invention also provides new methods and device structure to enable such progress.

The embodiments described in this invention, including the metal oxide, metal nitride, metal carbide or metal phosphide type semiconductors, with the width-reduced and/or thickness-reduced nanobridge structures, with the location or the number of the sensor molecule precisely controlled by biomolecule-blocking mask structures, can provide more reliable devices with enhanced signal-to-noise electrical signals. Such enhanced structures are also useful for DNA memory devices which require reading of stored DNA sequence information.

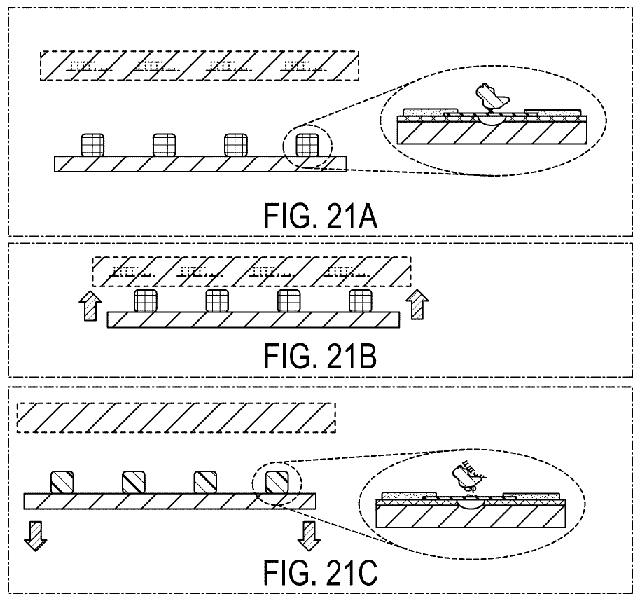

FIG. 21 is an exemplary embodiment illustrating memory applications of the disclosed technologies. A tethered array of encoded (memory written) DNA fragments is periodically positioned on a substrate as illustrated in FIG. 21(a), and when a polymerase single-DNA (or single-peptide) nanobridge array approaches DNA array being released as illustrated in FIG. 21(b), the DNA templates are taken up by polymerase array and the completed sensor array is moved array for DNA sequencing for memory information to be read the recorded as illustrated in FIG. 21(c).

### Embodiment 13—Optimization of Oxide Semiconductor Electrical Resistivity Toward Higher SNR

As stated, thinner and narrower oxide semiconductor nanobridges are generally more desirable to improve the semiconductor or FET properties and to increase the signal-to-noise-ratio SNR. This is because the electrical characteristics of the reduced volume nanobridge tends to accentuate the electronic pulse signal from the biosensing event, instead of burying the small signal.

In the case of biosensors using electrical signals as the basis of analyte detection, the SNR of detection is often dependent on the baseline electrical resistance (or impedance). For enhanced SNR of electrical or electronic based biosensors, one way of increasing the sensitivity is to reduce the dimension of the platform such as the bridge connecting two electrodes (source and drain electrodes), on which the tiny bioreceptor sensing molecules such as enzymes (e.g., DNA polymerase molecule), cells, antibodies, aptamers, peptides, or DNAs) are placed, with the sensing events comprising the singular or multiple attachment of analytes onto the bioreceptor molecule(s).

However, there may be limits to reducing the volume of the nanobridge (e.g., width and thickness) using the easily available processing methods. Therefore, an alternative means of increasing the electrical resistance is desirable. One way of accomplishing the increase in electrical resistance is to increase the intrinsic electrical resistivity of the nanobridge material. For oxide semiconductor nanobridge materials such as In₂O₃ (or more accurately, In₂O₃₋ₓ), the oxygen stoichiometry (or oxygen partial vacancy) plays an important role.

According to the invention, the oxygen vacancy is controlled in oxide semiconductors such as In₂O₃₋ₓ, with the value of x being less than 0.5, preferably less than 0.3, even more preferably less than 0.1 in order to provide higher SNR during biosensing. A similar approach of stoichiometry control to alter the electrical resistivity may be applied to other metal-nonmetal semiconductor nanobridges, such as made of indium tin oxide, indium zinc oxide, indium gallium zinc oxide, copper oxide, or other oxides, metal nitride semiconductors, metal phosphide semiconductors, metal sulfide semiconductors, or metal carbide semiconductors.

Such reduced oxygen vacancy may be obtained by, e.g., increasing the oxygen partial pressure during thin film sputter deposition or during post-deposition annealing of the semiconductor films or nanobridges. Therefore, the desired flow rate of oxygen gas during sputter deposition (or during evaporation deposition) may be at least 20% of the Ar gas flow rate, at least 60% of the Ar gas flow rate or at least 200% of the Ar gas flow rate. In the case of a fixed atmosphere with the flow of gases stopped and the sputter chamber sealed, the desired content of oxygen gas retained in the chamber during sputtering may be at least 20% of the Ar gas content, at least 60% of the Ar gas content or at least 200% of the Ar gas content. For a post-deposition annealing of sputtered or evaporated oxide semiconductor films, e.g., sputtered In₂O₃ films, a similarly high oxygen content atmosphere (i.e., at least 20%, preferably at least 60%, more preferably at least 200% more oxygen than Ar gas content) is employed for desirably higher electrical resistivity. Other parameters such as the bias voltage applied during sputtering may also be adjusted to increase the film resistivity. An ion implantation, e.g., oxygen atom implantation (followed by stress-relief anneal), may also be employed to force the oxygen stoichiometry to be increased. The following example describes the experimentally observed effect of oxygen partial pressure increase to desirably raise the electrical resistivity of In₂O₃ sputtered thin films.

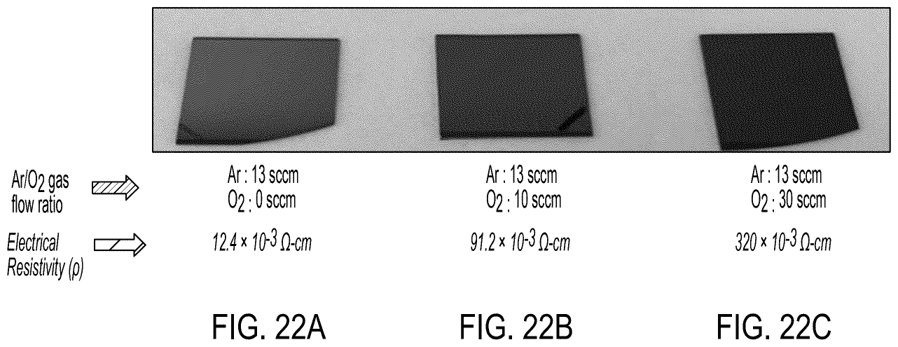

Example 1. Experimentally increased electrical resistance of In₂O₃ thin film. As an example of controlling the process to intentionally increase the electrical resistivity of oxide semiconductor films, an experiment was conducted using In₂O₃ thin films, as shown in FIG. 22. During the RF sputtering (RF power of about 200 Watt, the total sputtering time of about 10 minutes, the substrate of about 500 nm thick SiO₂ coated Si, substrate temperature of about 20° C.). By increasing the oxygen partial pressure in the sputtering chamber (i.e., by increasing the O₂ gas flow rate from zero standard cubic centimeter per minute (sccm) to 10 sccm, then to 30 sccm while maintaining the Ar gas flow rate constant at 13 sccm, it was possible to alter the electrical resistivity of the deposited In₂O₃ thin film (about 100 nm±10 nm thick) from about 12.4×10⁻³ Ω-cm (for zero sccm O₂) to about 91.2×10⁻³ Ω-cm (for about 10 sccm O₂), then to about 320×10⁻³ Ω-cm (for about 30 sccm O₂). For the high O₂ gas flow rate, the electrical resistivity increase was as much as by a factor of ×26.

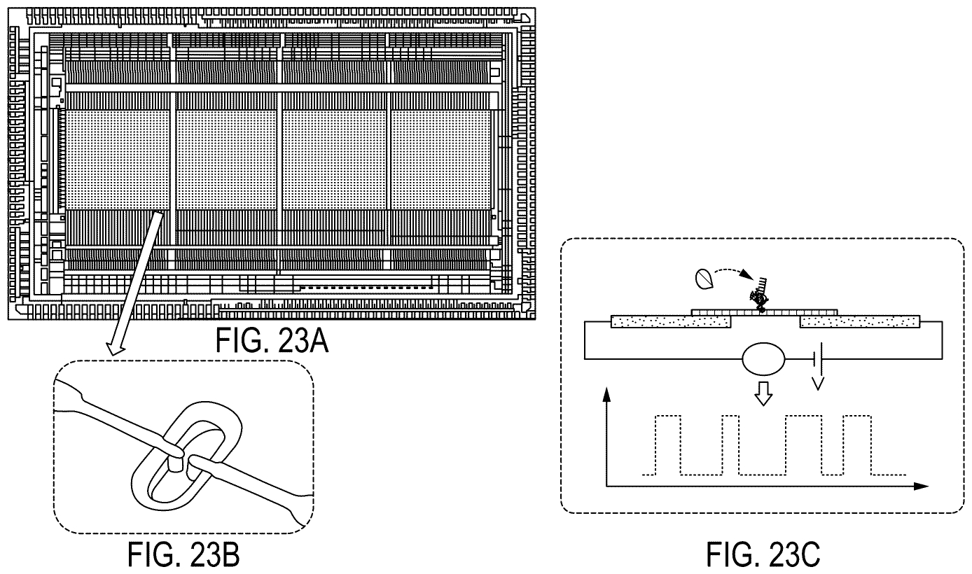

Example 2. Construction of CMOS platform nano-bio sensor array based on In₂O₃ nanobridge. Shown in FIG. 23 is an example CMOS platform nano-bio sensor array comprising an array of In₂O₃ nanobridge structure array onto which biosensor molecules are conjugate attached. Specifically, in FIG. 23(a), a CMOS chip array containing about 16,000 biosensors, each on In₂O₃ nanobridge is described. Nano-bio FET sensor structure on In₂O₃ nanobridge is illustrated in FIG. 23(b). In FIG. 23(c), the mechanism of electrical signal generation on binding and the disappearance of the signal upon unbinding of target analyte molecules onto the biosensor is described.

The indium oxide (In₂O₃) thin films may be deposited by various means such as sputtering, evaporation, atomic layer deposition (ALD), or chemical spin coating and baking of a precursor solution such as containing indium nitrate, indium chloride, or other soluble chemical compounds.

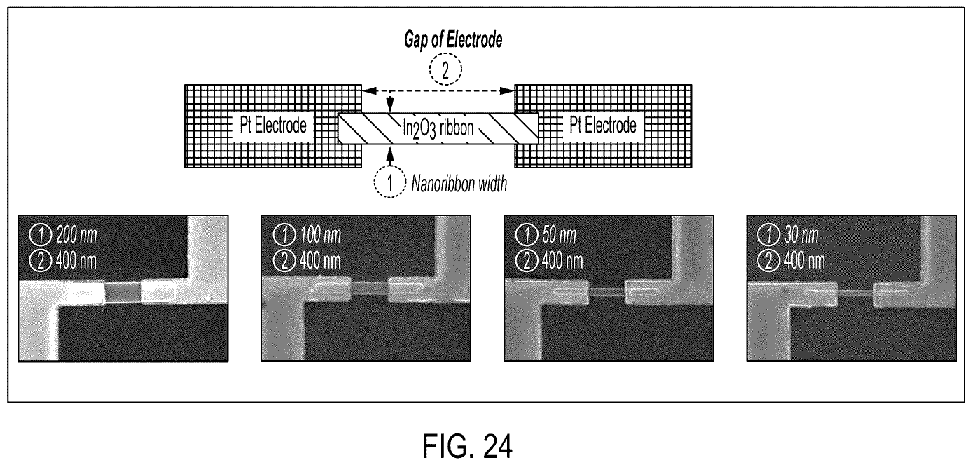

FIG. 24 shows fabrication of Indium Oxide (In₂O₃) nanoribbons of different dimensions on CMOS sensor chips. Specifically, FIG. 24 represents the scanning electron microscopy (SEM) micrographs showing In₂O₃ nanobridges e-beam lithography patterned into different bridge dimensions (with the width and length altered), which were fabricated from a sputter deposited indium oxide (In₂O₃) thin film with a thickness of about 10 nm. Such a different dimension of the nanobridge dictates the electrical resistance value and the area of biomolecule sensor attachments.

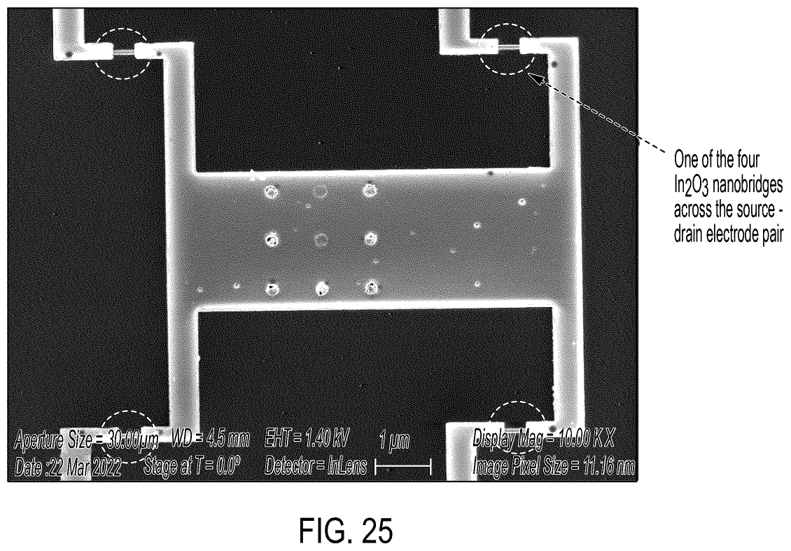

FIG. 25 shows an electron microscopy of In₂O₃ nanoribbons ribbon FET sensors arranged on a CMOS chip with 4-pixel quad electrode array.

Example 3—Functionalization of In₂O₃ nanobridge ribbons and attachment of Anti-His Aptamers.

Indium Oxide (In₂O₃) ribbons such as shown in FIG. 24 FIG. 25 were incorporated into CMOS-processed semiconductor chips having 16,000 nanosensor array in each about 0.5 cm ×1 cm sized chip by sputter deposition and patterned. The source and drain electrodes were made of thin film patterned Pt covering and anchoring the both ends of the In₂O₃ nanobridge ribbons. Such an architecture of In₂O₃ nanobridge placed below the electrode pair also has an additional beneficial function of restricting the exposed area of the In₂O₃ nanobridge so as to control the number of bioreceptor molecules attached on each sensor.

In order to attach the desired bioreceptor molecules on In₂O₃ nanobridge surface, the following functionalization steps were followed. The main functionalization chemistry incorporated a combination of (i) DNA APTES bioreceptor molecules, (ii) CBTF (sodium 4-((4-(cyanoethynyl)benzoyl)oxy)-2,3,5,6-tetrafluorobenzenesulfonate) as an amine-to-thiol coupling reagent for preparation of conjugates, and (iii) Thiols having the structure R—SH in which an alkyl group (R) is attached to a sulfhydryl group (SH). Instead of thiol, (APTES+NHS-DBCO+Azide combination) may also be utilized. APTES (3-Aminopropyl) triethoxysilane) is an aminosilane often used for silanization functionalization of surfaces with alkoxysilane molecules. It may also be used for covalent attaching of organic matter to metal oxides. NHS-DBCO Ester serves as an amine-reactive building block for click chemistry modification of amine-containing molecule for covalent bonds.

Methodology: 1. Clean the CMOS chips with twice IPA-Sonication processed. The chips were incubated with an APTMS:PTMS mixed solution with a ratio of 1:19, at 45° C. for 1 hr. APTMS ((3-Aminopropyl) trimethoxysilane) is an aminosilane used in silanization processes as a silane coupling agent. Propyltrimethoxysilane (PTMS) is utilized as a coupling agent and adhesion/bonding promoter. 2. Rinse the processed chips in ethanol twice (30 seconds each). 3. After drying with a stream of N₂ gas blow, the samples were cured at 70° C. in a vacuum oven for 1.5 h to accelerate cross linking of the APTES molecules on the surface. 4. Add 1 mM solution of CBTF (molecular weight of 421.26, with 0.7 mg CBTF dissolved in 170 ul of carbonate@PH 8.0 buffer to make 10 mM solution) and incubate for 30 min. 6. Add 28 ul of 0.6 uM solution of (thiolated)-B1.10-AptaHis1 and leave incubated at 10° C. for overnight. 7. Rinse chips twice with water next day before use.

Example 4—Construction of biosensor array on In₂O₃ nanobridge, and experimental measurements of binding assay electronic signals.

The need exists for simple and easy-to-manufacture molecular sensors that may yield molecular information with greater SNRs such that signals truly indicative of molecular binding interactions are distinguishable from non-informative noise. The example measurements described in this invention using In₂O₃ nanobridge configurations with biomolecular sensor attached on the nanobridge meet these unsolved challenges and needs. Advantages include ease of fabrication, high sensor yield, design supporting both single molecule and multi-molecule sensors.

In certain embodiments, the disclosure relates to sensors, systems including the sensors, and to methods of using the sensors and systems. Exemplary sensors may be used to, for example, detect the binding of a molecule of interest (herein encompassed by the term ‘target’) with the probe or a binding partner or ligand of the probe of the sensor. A variety of binding assays, including DNA-DNA hybridization, DNA-protein interaction, and protein-protein interaction are detectable using such nanobridge-based biosensor probes. A wide range of targets of interest (analyte molecules) including viruses such as COVID19, Hepatitis, HIV, Hepatovirus, and others may be detected.

When a target interacts with the probe, electrical changes in the circuit are sensed. These electrical changes, or informative electrical signals, may include current, voltage, impedance, conductivity, resistance, capacitance, or the like. In some examples, a voltage is initiated in the circuit and then changes in the electric current through the circuit are measured as substrates interact with the binding probe.

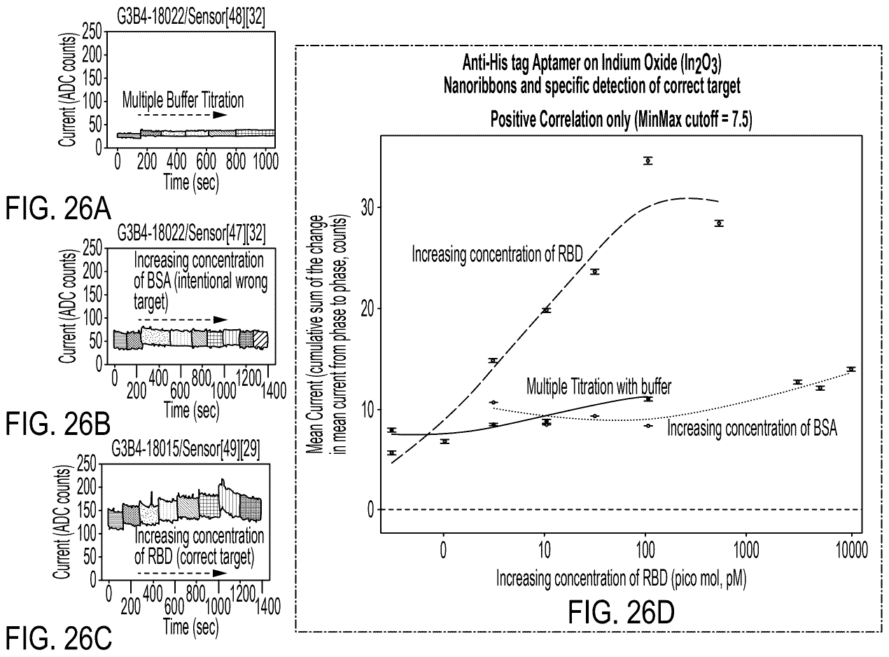

In the bioassay example of FIG. 26, an aptamer is attached to the indium oxide bridge surface functionalized by silane chemistry, to detect a nucleic acid-to-protein binding. The probe in this case is a DNA aptamer (a single-stranded DNA, of about 60 nt (nucleotide) in length) having affinity for Histidine-tag on a protein. In the presence of the target Receptor Binding Domain (RBD) of COVID19 that contained a His-tag at its termini, the sensor current exhibits aptamer-protein binding events. The average current level increases with increasing target molecule concentration. Plotting the mean current (cumulative sum of the change in mean current from phase to phase against the target concentration) produces binding response curves. For comparison, the negative controls in the form of either assay buffer only or a non-His tag protein Bovine Serum Albumin (BSA) was titrated on separate chips. The mean current response curve for the two negative controls is at the basal level in comparison to the specific target His-RBD.

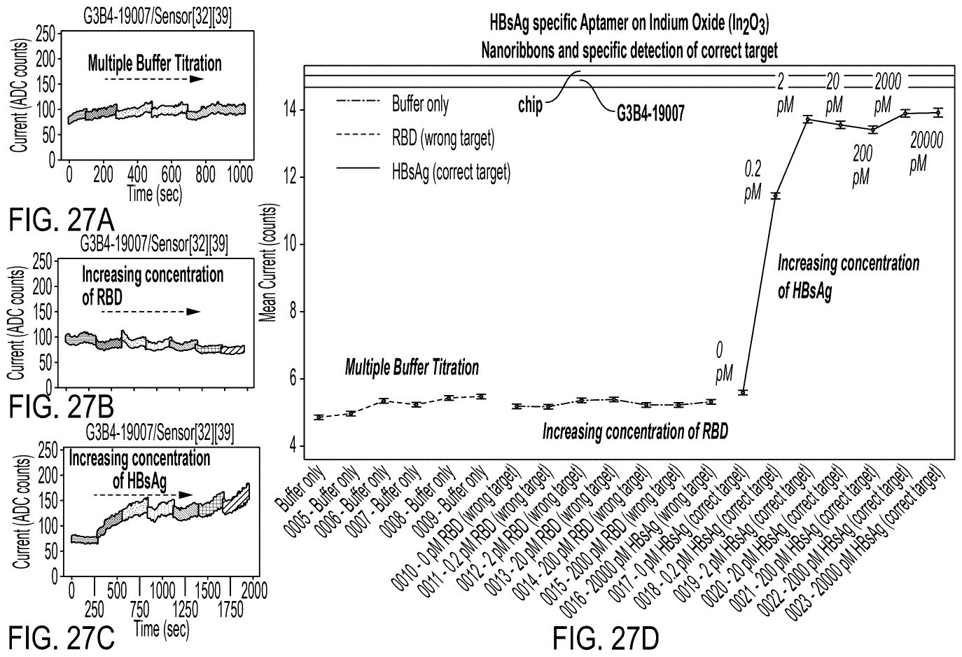

In FIG. 27 demonstrates model binding assays on a different aptamer-protein interaction. In this case, an aptamer probe is attached to the Indium oxide bridge functionalized by silane chemistry. The probe is a DNA aptamer (a single-stranded DNA, of about 51 nt in length) having affinity for Hepatitis B surface Antigen (HBsAg). The negative controls in the form of either just assay buffer titration or a non-specific protein RBD were tested on the same chip. The mean current response curve for the two negative controls is at the basal level. However, in the presence of the right target HBsAg, the sensor current exhibited a significant increase in the average current level with increasing target concentration. Plotting the mean current (cumulative sum of the change in mean current from one phase to the next phase) against the target concentration produced binding response curve.

### Embodiment 14. Increase of Oxide Semiconductor Electrical Resistivity by Reduction of the Semiconductor Volume in the Nanobridge Using Intentional Porosity

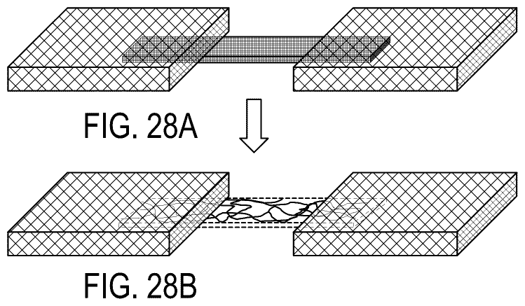

FIG. 28 schematically illustrates resistivity increase in nanobridge of In₂O₃ and related metal-nonmetal semiconductors by porous structure. FIG. 28(a) shows In₂O₃ rectangular nanobridge with near full density, positioned under the source-drain metallic electrodes and FIG. 28(b) similarly positioned but porous nanobridge with higher electrical resistivity.

The metal-nonmetal semiconductor layer (such as the In₂O₃ semiconductor thin film) in FIG. 28(a) may be deposited by physical vapor deposition like sputtering or evaporation, or by chemical vapor deposition, electrodeposition, or sol-gel type precursor spin coating and baking, followed by nanopatterning. The nanobridge may be positioned below the metal electrodes, which may be achieved by depositing the In₂O₃ film first, followed by patterning into a nanobridge shape, then metal electrode deposition and patterning. The nanobridge may alternatively be positioned above the electrode pair, either with planarizing dielectric underneath, or by stamping pattern transfer of the pre-patterned nanobridge onto the electrode gap region.

Desired thickness of the In₂O₃ rectangular nanobridge is in the range of e.g., about 1-20 nm thick, 3-10 nm thick or 3-5 nm thick. Desired width of the nanobridge is in the range of 5-40 nm wide, 5-20 nm wide or 5-10 nm wide. When the thickness and width of the nanobridge are not made small enough, the electrical conductivity might be too high (with the electrical resistivity being too low). In such a case, it may be desirable to intentionally increase the resistivity. One way of achieving a higher electrical resistivity for the same nanobridge overall dimension is to intentionally make the nanobridge film porous, as illustrated in FIG. 28(b).

Such porous film structure may be obtained by oblique incident angle sputtering or evaporation. Alternatively, such a porous structure may be achieved by co-deposition of In₂O₃ with other dissolvable metal or dissolvable oxide using co-sputtering, co-evaporation, or co-electrodeposition. Examples of easily dissolvable metals include Cu, Zn, Sn, Sb, Fe, Ni, Co, Mn, Si, Mo, Ta, Hf or their alloys. Examples of easily dissolvable oxides include CuO, ZnO, SnO₂, Sb₂O₃, Fe₂O₃, NiO, CoO MnO₂, MoO, MoO₃, Ta₂O₃, HfO₂, SiO₂, and so forth, followed by selective chemical etch removal (or reactive ion etch removal) of metallic or oxide component material. Instead of metallic or ceramic inclusions, a carbon-containing material such as graphite may also be incorporated, e.g., by co-sputtering, which may easily be burned away by heating, e.g., at about 300-500° C., optionally with occasional vacuum pumping to remove the burn product such as CO or CO₂ gas.

The desired degree of porosity may be at least 20%, at least 50% or at least 70%. porosity may be as high as 85% if the percolation paths are semi-aligned along the fiber length direction. The electrical resistivity of the metal-nonmetal semiconductor is consequently increased by the porous structure by at least 20%, at least 50%, or at least 70%.

### Embodiment 15. Increase of Oxide Semiconductor Electrical Resistivity by Introduction of Insulating Ceramic Particles in the Nanobridge Semiconductor Volume

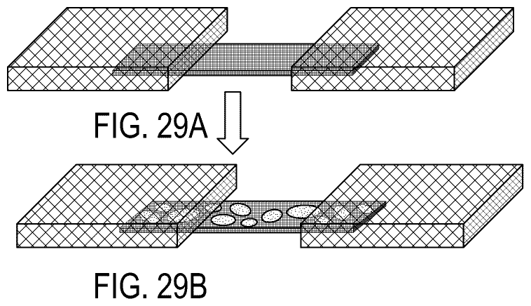

FIG. 29 shows resistivity increase in nanobridge of metal-nonmetal compound (e.g., In₂O₃ nanobridge) by intentional inclusion of insulating ceramic for volume dilution according to one embodiment of the disclosure. It may be desirable to have the dimension of the FIG. 29(a) type nanobridge made small so as to avoid a situation of too conductive nanobridge. When the thickness and width of the nanobridge are not made small enough, the electrical conductivity might be too high (with the electrical resistivity being too low). In such a case, it is desirable to intentionally increase the resistivity. One approach of intentionally increasing electrical resistivity for the same dimension of nanobridge is to embed the bridge material with insulating ceramic particles (such as oxide, nitride or fluoride) so that the total In₂O₃ type semiconductor material in the nanobridge is diluted, as illustrated in FIG. 29(b). The desired thickness of the insulator particle embedded In₂O₃ rectangular nanobridge may be in the range of about 1-20 nm thick, 3-10 nm thick or about 3-5 nm thick. The desired width of the insulator particle embedded In₂O₃ nanobridge is in the range of about 5-40 nm wide, 5-20 nm wide or about 5-10 nm wide.

For this purpose, the In₂O₃ rectangular bridge material may be filled with insulating second phase oxide islands (such as Al₂O₃, SiO₂, MgO, Ta₂O₅, Cr₂O₃, rare earth oxide), or nitride islands (such as by AlN, Si₃N₄, rare earth nitride), or fluoride islands (such as MgF₂, CaF₂, AlF, rare-earth fluoride), and so forth. If there is a sufficient amount of the embedded insulating ceramic island material, there will be e.g., only 20-30% volume of In₂O₃ matrix material left by dilution, for much reduced conductivity. The insulating particle is selected from elements that have little or no solubility or alloying behavior with In₂O₃ type semiconductor compound.

Such semiconductor-insulator composite structured In₂O₃ film structure may be obtained by e.g., two-target (e.g., two metal reactive sputtering, or two oxide target co-sputtering) or co-evaporation process. The desired volume fraction of the insulating ceramic may be at least 20%, greater than 50%, greater than 70% or greater than 85%.

### Embodiment 16—Use of Molecule Anchoring Island Array for Single Molecule Bioreceptor Structure on Metal-Nonmetal Semiconductor Nanobridge

It is desirable to ensure that the bioreceptor sensor molecules (such as polymerase molecules, DNAs, aptamers, peptides, and so forth) are separated from each other without agglomeration of two or more molecules nearby. Such agglomeration will cause complications in polymerase-DNA interactions for proper signal discrimination and accurate sequencing. For single polymerase molecule attachment, the use of anchoring islands is highly desirable, according to the invention. Noble metal islands (such as Pt, Pd, Au, Ag, Ru, Rh and their alloys) tend to strongly and covalently bind with some linker or conjugation molecules (e.g., thiol related binding) onto which the polymerase molecule or other bioreceptor sensor molecules may easily be attached using a number of binding or conjugation moieties). If the anchoring island size is desirably made very small, with the anchoring island area on the order of the polymerase molecule size (e.g., about 5 nm, or more generally about 3-10 nm equivalent diameter), the formation of multiple-polymerase agglomeration onto the same anchoring island may be minimized. The anchoring islands have to be discontinuous from each other in order to avoid electrical shorting effect of noble metals.

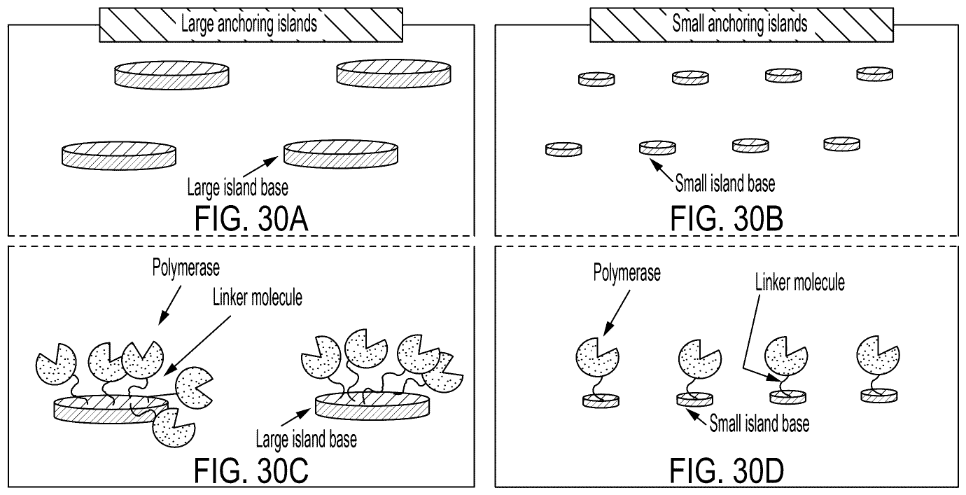

Schematically illustrated in FIG. 30 is a comparison of large size anchoring islands (e.g., >30 nm equivalent diameter) vs. small, size-limited nanoscale anchoring islands (e.g., 5 nm). Large diameter anchoring islands, FIG. 30(a) may cause an undesirable, multiple polymerase molecule binding (or other multiple bioreceptor sensor molecule binding), like in FIG. 30(c). Size-limited, nano-island array, FIG. 30(b), with the equivalent diameter of each anchoring island comparable to the average diameter of polymerase (about 5 nm), aptamer, DNA or peptide of similar regime of sizes, essentially ensures the attachment of a single molecule bioreceptor sensor molecule, FIG. 30(d).

In one embodiment of the disclosure, the linker molecules (or conjugations) that connect the polymerase molecule to the base anchoring islands may be selected from various binding moieties, e.g., thiol binding, biotin-streptavidin, antigen-antibody, pyrene-maleimide, maleimide-azide, amine-related, silane related complexes, PEG (polyethylene glycol) related complexes, and so forth. The length of the linker molecules is desirably kept to a small dimension (e.g., less than about 200 nm, less than about 50 nm, less than about 20 nm or less than about 10 nm) so as to minimize any inadvertent effect of linker length on electrical sensing signals. With such a thiol type covalent and strong binding of bioreceptor single molecule, some functionalizing (like silane based) could be omitted for easier, more reliable sensor structure assembly.

The comparison of a large diameter anchoring island base vs small diameter anchoring island base in FIG. 30 describes the critical need to have the anchoring island area to be limited to the regime of polymerase diameter. Sometimes if the concentration of the polymerase supplied in the sequencing cocktail solution is excessive, even a small 10 nm metal island may harbor multiple thiol-end linkers. Therefore, a two-linker design is optionally adopted to further ensure a single molecule attachment. In a two-linker system, both linkers may use PEG (polyethylene glycol) backbones, the first linker has a thiol group (facing metal island) and a biotin at the other (facing streptavidin) while the second linker has a biotin group (to streptavidin) and a maleimide group at the other (to cysteine at the N- or C-terminal of polymerase). The size of streptavidin tetramer is about 10 nm.

To minimize the close presence of multiple polymerase molecules, according to one embodiment of the disclosure, the concentration of polymerase in the cocktail solution may also be adjusted. The desirable number of polymerases for each anchoring island is about 0.5-2, about 0.5-1 or about 0.7-1.

The desirable range of the anchoring nano-island diameter in FIG. 30(b) is about 5-30 nm, less than about 15 nm, less than about 10 nm or less than about 6 nm. Such a size limited islands will prevent or minimize undesirable multiple molecule binding. If multiple bioreceptor sensor molecules are desired (e.g., for biding assays for detection of disease-causing virus), the anchoring island array may be periodic or random even though a periodic arrangement may be preferred. The spacing between neighboring anchoring islands is desirably about 20 nm, at least 100 nm or at least 500 nm.

The anchoring island base is preferably made of small size and thin metallic nanodisk, for example, with a thickness of about 1-5 nm thick Pt, Au, Pd, Ag, Ru, Rh type noble metal or their alloy film, patterned to be sufficiently small to allow only a single bioreceptor sensor molecule (such as polymerase molecules, aptamers, DNAs, peptides) to be attached on each island. Such noble metal surfaces may be desirable for strong bonding (e.g., using covalent thiol-related bonding). An optional adhesion layer such as 1-5 nm thick Ti, Cr, Hf, Zr may also be used to minimize detachment of the island from the metal-nonmetal semiconductor substrate surface.

The linker molecules (or conjugations) that connect the polymerase molecule to the base islands may be selected from various binding moieties, for example, thiol binding, biotin-streptavidin, antigen-antibody, pyrene-maleimide, maleimide-azide, amine-related or silane related complexes. The length of the linker molecules is desirably kept to a small dimension, e.g., less than about 200 nm so as to allow evanescent field microscopy detection and fluorescent signal capture of nucleotide incorporation event. In certain embodiment, the length of the linker molecule may be less than 50 nm, less than about 20 nm or less than about 10 nm.

If neighboring polymerase molecules are too closely positioned, as illustrated in FIG. 30(c), there may be a possibility of two or more neighboring polymerases interacting with the same DNA molecule at different sites, which may interfere with the polymerase enzyme sequencing operation, producing undesirable complications and inaccurate sequencing analysis. Therefore, in one application, it is preferred to have a spaced-apart polymerase array. However, in order to have a high speed output, a greater number of single molecule polymerase locations within a given sample analysis field is highly desirable. For these purposes, having periodic or nearly periodic, yet individually well-separated islands of polymerase is highly desirable.

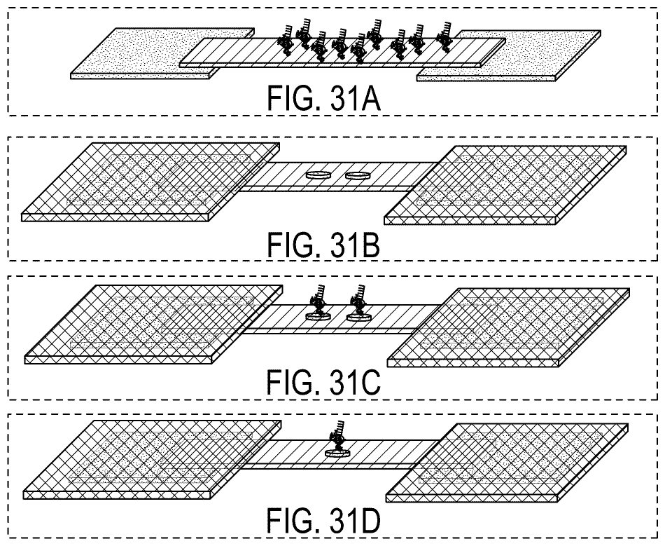

FIG. 31 schematically illustrates the use of noble metal anchoring islands for precise control of number and location of bioreceptor molecule attachment according to one embodiment of the disclosure. FIG. 31 illustrates an advantageous use of noble metal anchoring islands for precise control of number and location of bioreceptor molecule attachment (e.g., sensor molecules such as aptamers, polymerase enzyme molecules, DNAs, peptides) on In₂O₃ bridge surface according to one embodiment of the disclosure. Without anchoring islands, uncontrolled, multiple bioreceptor sensor molecules are attached onto each In₂O₃ bridge surface, FIG. 31(a). While such a multiple sensor attachment may be useful for binding assay type detection of disease-causing antigens, metabolic status, or physiological parameters, it is not a preferred structure for DNA or genome sequencing. In FIG. 31(b), two noble metal anchoring islands are intentionally placed to obtain only two bioreceptor sensor molecules as illustrated in FIG. 31(c). The anchoring island may be shaped as a circle, an oval, a square, or a rectangle. With only one anchor islands placed on a single bioreceptor molecule a single polymerase enzyme is allowed to attach to the bridge surface as shown in FIG. 31(d). This structure may present an ideal detector for accurate DNA or genome sequencing. In FIG. 31(d), only a single anchoring island is provided to allow a single-molecule biosensor to attach for DNA or genome sequencing, in which unambiguous electrical signal is desired for each nucleotide attachment event. For redundancy and other quality control purposes, as well as for consideration of probe molecule attachment efficiency or for multi-type probe array, a two-molecule biosensor per bridge, a three-molecule biosensor per bridge, or up to ten-molecule biosensor per bridge may be utilized for various biosensing applications.

### Embodiment 17—Fabrication Techniques for Sub-10Nm Anchoring Islands

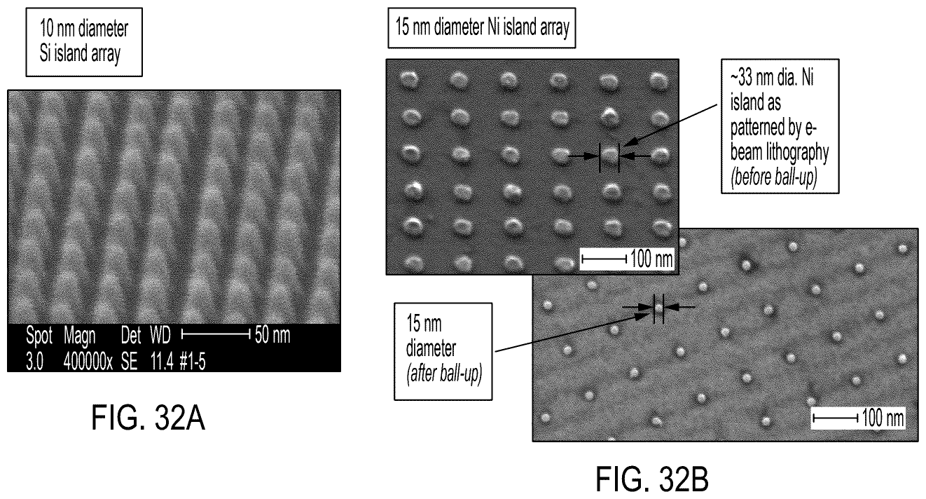

For single molecule such as single polymerase bioreceptor sensor array, sub-10 nm anchoring islands must be fabricated through nanofabrication. Formation of such desirably small islands is not easy. Special techniques, such as electron-beam lithography, EUV (extreme UV) lithography, laser lithography, nano-imprint lithography, or other nanofabrication methods may be required. In certain embodiment, the disclosure relates to new techniques for preparing sub-10 nm island structures, including a sub-10 nm nanoimprinting stamp creation. An example technique to form such small diameter, periodic (or near periodic) array of islands is schematically illustrated in FIGS. 32-34.

FIG. 32 shows examples of nanofabricated nano-island array according to the disclosed embodiments. Specifically, in FIG. 32(a), a periodic 10 nm diameter Si nano-island array obtained by electron-beam lithography is shown. HSQ (hydrogen silsesquioxane) negative resist was utilized to obtain the Si nano-island array. In FIG. 32(b), a 15 nm (approximately) diameter Ni metal island array was formed by e-beam lithography to about 33 nm diameter, a few nm thick Ni film disk array, followed by ball-up annealing at about 400° C. to intentionally reduce the island diameter to about 15 nm. Such a ball-up process occurs by natural thermodynamic phenomenon of surface-energy-reducing microstructural change. By such a ball-up process (e.g., starting with a 15-20 nm diameter e-beam patterned flat metal film followed by annealing), a 10 nm or sub-10 nm diameter Au, Pt, or Pd anchoring island array (at any desired spacing in the range of e.g., 50 nm-20 um) may also be fabricated for binding of single polymerase molecule (about 3-10 nm diameter) per each metal island, as desired for single molecule enzyme based sequencing.

### Embodiment 18—Use of Small Dimension Nanoimprint Lithography to Rapidly Manufacture Many Devices with Sub-10 Nm Anchoring Island Array for Single Polymerase Binding

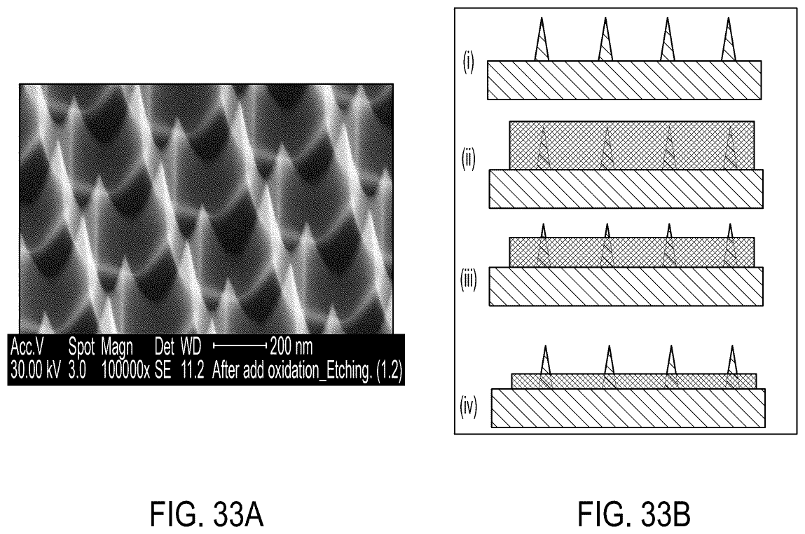

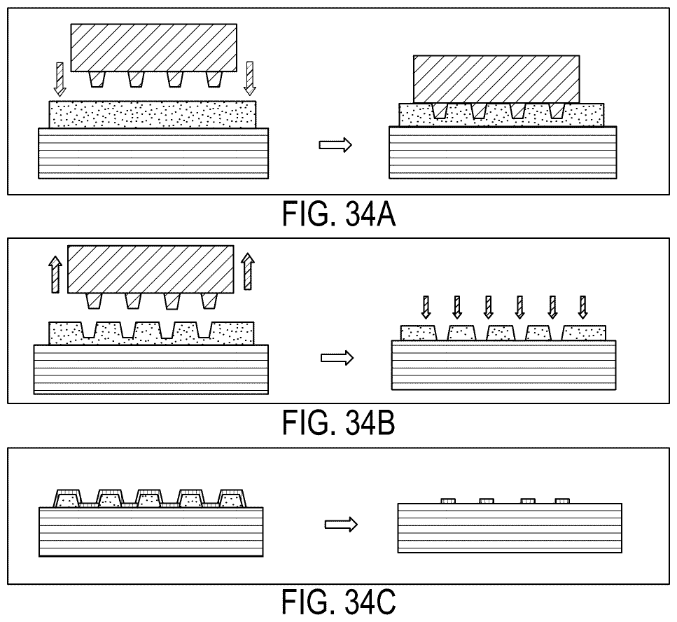

While e-beam type lithographic processing may be utilized to obtain near or sub-10 nm diameter anchoring island array, such e-beam type lithography is slow and expensive. For massive applications of genome or DNA sequencing, sometimes thousand sequencing systems need to be distributed to various users such as medical centers, hospitals, or industrial biomedical institutes. Therefore, an inexpensive nanofabrication processes for sub-10 nm, periodic anchoring islands are highly desirable. In certain disclosed embodiments, a novel method for fabricating desirable sub-10 nm nanoimprint mold is disclosed which may be used to form sub-10 nm diameter anchoring island array (made of e.g., Au, Pt, Pd, Ag, Ru. Rh or their alloys) for binding of a single polymerase molecule per each metal island, as desired for single molecule enzyme-based sequencing. This method is illustrated in FIGS. 33-34.

An example nanoimprint process to obtain sub-10 nm island is described in FIG. 33. First an extremely sharp Si nanocone array (e.g., about 2 nm or less tip radius) is fabricated by chemical etching and oxidation repeat process of Si nanopillar array (e.g., obtained by standard lithography steps) as shown in FIG. 33(a). In one embodiment, the nanocone structure is first planarized by resist coating of HSQ (hydrogen silsesquioxane) followed by conversion into SiO₂ via slow low-temperature annealing 0 Next, chemical-mechanical-polishing (CMP) is applied to planarize the surface and SiO₂ is then selectively partially etched to obtain a protruding nanoimprinting pillar array mold as shown in FIG. 33(b). The Si nanoimprint mold is then utilized to produce a sub-10 nm (e.g., 5-8 nm diameter) metal island array by imprinting, RIE etch, metal film deposition and lift-off process as illustrated in FIG. 34.

FIG. 34 schematically illustrates an exemplary nanoimprinting operation to form a sub-10 nm metal anchoring island array using a 5-8 nm diameter tip arrayed imprinting mold. FIG. 34(a) schematically shows nano imprinting onto a resist layer. FIG. 34(b) shows an array pattern formed by reactive ion etch (RIE). FIG. 34(c) shows the process of metal film deposition and lift-off to obtain a metal island array.

### Embodiment 19—Multiplexing Biosensor Array Utilizing Droplet Array Sensing Structure

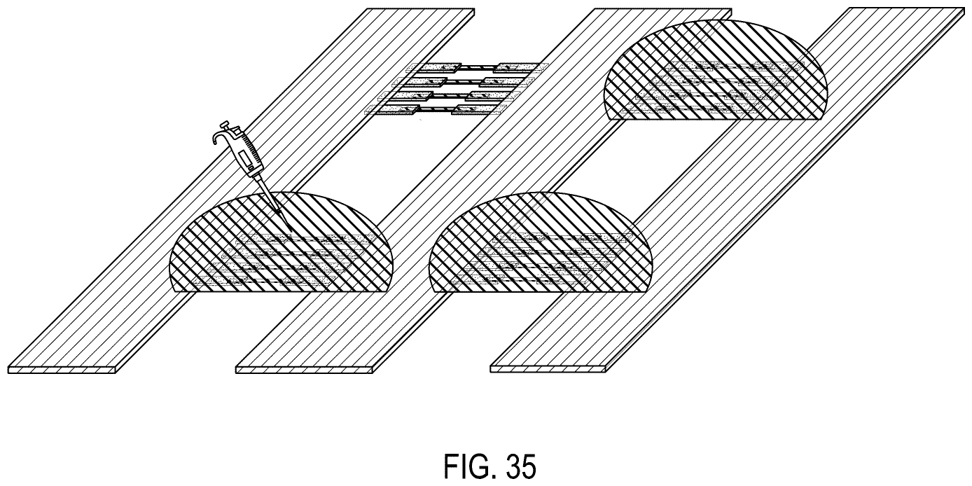

For some biosensing applications, multiplexing sensing, (i.e., simultaneous biosensing of multiple samples for different patients), different types of disease assays, or from different body fluids. Shown in FIG. 35 is a device structure that allows such a multiplexing biosensing using multiple regions of analyte droplet solutions (e.g., for separate assay or sequencing with different patient's samples, or the same patient's samples for different types of analysis).

The metal-nonmetal semiconductor sensors (e.g., In₂O₃ type nanobridge sensors) are positioned between the source-drain electrodes. Each In₂O₃ nanobridge contains a single bioreceptor sensor molecule for sequencing (or multiple sensor molecules if desired for disease assay type analysis). The nanobridges are grouped into different sections for multiplexing bioanalysis, e.g., for simultaneous analysis of different patients' samples or the patient's samples for different types of analysis.

In one application of the disclosed principles, separate droplets of solutions for bioreceptor molecule binding are placed at different sections to attach different bioreceptor sensor molecules as desired (e.g., different aptamers, polymerase, DNAs, peptides), and the solution may be washed away after the bioreceptor molecules are attached. The droplet placement may be carried out by using micropipette or inkjet release head, e.g., comprising single or multiple release micro nozzles or nano nozzles. Next another set of droplets comprising analyte solutions (or sequencing analysis solutions) may be placed at different sections for assays or sequencing analysis. The droplet cocktail solutions may contain water, salts, patient's samples like blood, saliva, perspiration, urine, or other types of body fluid samples, or animal samples. For sequencing analysis, the cocktail solution may comprise nucleotides, salts, and other chemicals, catalysts, and so forth.

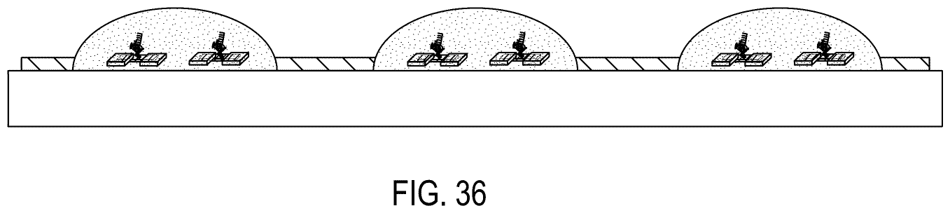

Optionally, hydrophobic coating may be added and patterned except the active sensor sections so that the water droplet formation is made easier with less lateral solution spread, as illustrated in FIG. 36. The separation of neighboring droplets may be obtained either by physical separation or by use of a patterned hydrophobic mask coating with hydrophilic openings. Such droplet array configurations allow sensor signal data to be obtained, analyzed and sequenced from different analyte samples (droplets) of the same type, from different analyte samples of the different type, or from different patient's analyte samples. The hydrophobic coating material is patternable, and may be selected from e.g., PTFE (Teflon), fluorine-containing polymers, rare-earth oxide layers (e.g., CeO₂, Yb₂O₃), or other polymeric or oxide materials with high water-contact angles, e.g., greater than 60 degrees, greater than 90 degrees or greater than 120 degrees.

A micropipette (or nanopipette) array or inkjet printing head type release nozzle array may be used to release a spaced-apart biological solution small droplets (e.g., 0.1 to 1 uL volume, about 500 um to 1 mm size) to produce a high-density, sequencing or biosensing array platform. Within each droplet of analyte solution, an array of attached bioreceptor molecules (e.g., DNA, RNA, peptide, aptamer, polymerase) may be present.

### Embodiment 19—Multiplexing Biosensor Array Structure Via Sub-Division of Sensor Array Using Compartments

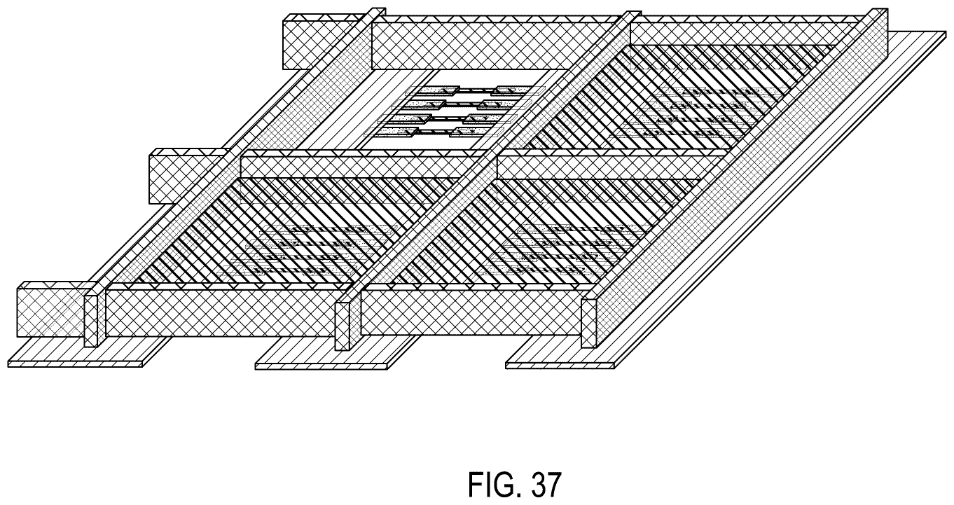

Yet another way of introducing the multiplexing capability is to subdivide the device platform to allow separate biosensor analysis from each compartment, as illustrated in FIG. 37. In FIG. 37, the metal-nonmetal semiconductor sensors (e.g., In₂O₃ type nanobridge sensors) are positioned between the source-drain electrodes. Each In₂O₃ nanobridge contains a single bioreceptor sensor molecule for sequencing (or multiple sensor molecules if desired for disease assay type analysis). The nanobridges are grouped into different compartments, which provides sub-divided nano sensor array for multiplexing bioanalysis, e.g., for simultaneous analysis of different patient's samples or the same patients samples for different types of analysis. The desired size of each compartment is in the range of 100 nm to 2 mm size, preferably 200 nm to 500 um size, more preferably 500 nm to 10 um size), made of nano/micro fabricated walls of ceramic or polymer material, e.g., silica, alumina, PMMA (polymethyl methacrylate) or other micro-patternable polymers.

Separate droplets of solutions for bioreceptor molecule binding may be placed into different compartments to attach different bioreceptor sensor molecules as desired (e.g., different aptamers, polymerase, DNAs, peptides), and the solution may be washed away after the bioreceptor molecules are attached. The solution placement into each compartment may be performed by using micropipette or inkjet release head, e.g., comprising single or multiple release micro nozzles or nano nozzles.

Then another set of solutions comprising analyte solutions (or sequencing analysis solutions) may be placed into different compartments for assays or sequencing analysis. The cocktail solutions in separate compartments contain water, salts, patient's samples like blood, saliva, perspiration, urine, or other types of body fluid samples or animal samples. For sequencing analysis, the cocktail solution comprises nucleotides, salts, and other chemicals, catalysts, etc.

### Additional Exemplary Embodiments

Article/Device type Examples—Example 1 is directed to an article comprising one or more biosensor structures, each biosensor structure comprising: A nanoelectrode pair of source electrode and drain electrode made of a conductive metal, with the electrodes separated by a nanogap; At least one nanobridge made of a metal-nonmetal compound semiconductor connecting the two electrodes across the nanogap; One or more bioreceptor biomolecules attached onto the semiconductor nanobridge surface to attract and bind with one or more target molecules or target biomolecules; One type or more analyte solutions comprising water, salts, the target molecules to be detected, placed in a biosensing reaction chamber or a microfluidic reaction chamber; Electronic circuit system and computer data analysis system for measurements and analysis of electronic sense signals; Biosensor function performed based on an attachment or detachment of one or more target biomolecules onto or from the biosensor, which alters the electrical characteristics of the semiconductor nanobridge to produce electronic pulse signals to enable a detection of the target molecule.

Example 2 is directed to the article of Example 1 wherein the nanobridge made of metal-nonmetal compound semiconductor is selected from an oxide, carbide, nitride, phosphide or sulfide semiconductor.

Example 3 is directed to the article of Example 1 wherein the nanobridge is made of semiconducting oxide, with the nanobridge oxide material selected from n-type semiconducting oxides (such as ZnO, In₂O₃, SnO₂, (In,Sn)₂O₃ (ITO type), NbO₂, In—Ga—O (IGO), In—Zn—O (IZO), In—Ga—Zn—O (IGZO), In—Al—Zn—O (IAZO), TiO₂, WO₃, and various doped versions of these oxides.

Example 4 is directed to the article of Example 1 wherein the nanobridge is made of semiconducting oxide, with the nanobridge oxide material selected from p-type semiconductors (such as CuO, Cu₂O, NiO, spinel oxides, CuMO₂ (M=Al, Ga, In, such as CuInO₂, CuAlO₂, CuGaO₂, CuSrO₂, SrCu₂O₂), LaCuOS, LaCuOSe, with various types and concentrations of dopants added as needed.

Example 5 is directed to the article of Example 1 wherein the conducting electrode is made of highly conductive metallic materials selected from Au, Pt, Pd, Ag, Ru, Rh, Ir, Cu, Ni or their alloys.

Example 6 is directed to the article of Example 1 wherein the bioreceptor biomolecule is bound to the nanobridge surface by one or more inserted conjugating moiety molecules selected from thiol binding, biotin-streptavidin complex, antigen-antibody complex, pyrene-maleimide complex, connecting moiety comprising maleimide-azide, azide-related click chemistry, amine-related click chemistry, tag-binding related links, silane-related, or PEG (polyethylene glycol) related complexes.

Example 7 is directed to the article of Example 1 wherein the bioreceptor biomolecule is selected from DNAs, peptides, aptamers, enzyme molecules, DNA Taq polymerase single enzyme molecule, cells, or antibodies.

Example 8 is directed to the article of Example 1 wherein the target analyte molecules comprise one or more disease-inducing or epidemic-causing virus (such as coronavirus including COVID-19 virus, SARS or influenza virus, hepatitis, HIV, hepatovirus), or other micro bacteria and organisms, nucleotides in DNA analysis, proteins or DNAs in forensic analysis, enzymes, hormones, albumin, peptides for health examinations, sensing of environmental pollutants (organic or inorganic) including volatile organic compounds.

Example 9 is directed to the article of Example 8 wherein the target molecules are part of human or animal biofluids samples, selected from blood, sweat, saliva, nasal mucus, endocrine glands, or urine.

Example 10 is directed to the article of Example 8 wherein the target molecules are DNAs, proteins, bacteria, or virus present in forensic samples prepared into a liquid, gas or vapor form for more accurate analysis.

Example 11 is directed to the article of Example 8 wherein the target molecules are inorganic or organic environmental pollutants present in environmental water, air, or soil samples prepared in the liquid, gas or vapor form test samples.

Example 12 is directed to the article of Example 1 wherein the semiconductor nanobridge is in a thin ribbon configuration with a nanoscale dimension of less than 200 nm in width, preferably less than 50 nm, more preferably less than 20 nm, even more preferably less than 10 nm.

Example 13 is directed to the article of Example 1 wherein the thickness of the nanobridge is less than 50 nm, preferably less than 20 nm, more preferably less than 10 nm, even more preferably less than 2 nm.

Example 14 is directed to the article of Example 1 wherein the biosensor additionally has one or more gate electrodes present near the nanoelectrode pair as a top-gated, bottom-gated, parallel-gated, perpendicular-gated or at-angle gated configuration.

Example 15 is directed to the article of Example 1 wherein the biosensor is an insulator-gated field effect transistor biosensor comprising an ultrathin, anticorrosion dielectric layer cover on which the sensor biomolecule is attached.

Example 16 is directed to the article of Example 15 wherein the ultrathin, anticorrosion dielectric layer cover is selected from SiO₂, Si₃N₄, Al₂O₃, ZrO₂, or HfO₂.

Example 17 is directed to the article of Example 15 wherein the thickness of the ultrathin anticorrosion dielectric layer cover is in the range of 0.2-50 nm, preferably 0.5-10 nm, more preferably 0.5-5 nm.

Example 18 is directed to the article of Example 1 wherein the nanobridge has a multitude of bioreceptor molecules attached on its surface.

Example 19 is directed to the article of Example 1 wherein the nanobridge has a restricted number of bioreceptor molecules attached on its surface at pre-selected open locations in the biomolecule-attachment-preventing mask layer, preferably less than 20 bioreceptor molecules per nanobridge, more preferably less than 5, even more preferably less than 3.

Example 20 is directed to the article of Example 1 wherein the nanobridge has a single pre-selected open location in the biomolecule-attachment-preventing mask layer, allowing only a single bioreceptor sensor molecule attached on the nanobridge.

Example 21 is directed to the article of Example 1 wherein the nanobridge is covered by a dielectric mask that blocks the attachment of bioreceptor molecules, with one or more openings which allow the attachment of bioreceptor molecules only in the opening area, so as to restrict the number of bioreceptor molecules attached on its surface.

Example 22 is directed to the article of Example 21 wherein the dielectric mask material is selected from a polymeric material, a ceramic material or a composite material nanopatterned into nanoscale opening of circular, square, rectangular or irregular shape on each nanobridge, or a slit-type opening spanning multiple nanobridges, for selective bioreceptor molecule attachment.

Example 23 is directed to the article of Example 21 wherein the dielectric mask material is selected from of polymethylmethacrylate (PMMA), SU-8, polytetrafluoroethylene (PTFE), polyethylene glycol (PEG), polydimethylsiloxane (PDMS), related elastomers, polystyrene (PS), and polyurethane, or SiO₂, Al₂O₃, MgO, TiO₂, ZrO₂, or Ta₂O₅ layer, with a thickness of less than 200 nm, preferably less than 100 nm, more preferably less than 20 nm, even more preferably less than 5 nm.

Example 24 is directed to the article of Example 21 wherein the opening in the mask for bioreceptor molecule attachment has an average diameter of less than 500 nm, preferably less than 50 nm, more preferably less than 10 nm, even more preferably less than 6 nm.

Example 25 is directed to the article of Example 21 wherein the slit-type opening for bioreceptor molecule attachment has a width of opening less than 500 nm, preferably less than 50 nm, more preferably less than 10 nm, even more preferably less than 6 nm.

Example 26 is directed to the article of Example 19 wherein the pre-selected open locations on the nanobridge selectively allows different types of bioreceptors by virtue of their surface pre-treated or pre-coated with one or more functionalities to enhance attachment of specific probe molecule types on the specific desired open hole locations.

Example 27 is directed to the article of Example 1 wherein the source electrode and drain electrode positioned above the nanobridge serves as a mask to restrict the number of bioreceptor molecules attached on the nanobridge surface.

Example 28 is directed to the article of Example 27 wherein the source electrode and drain electrode are positioned close to each other with a reduced gap spacing so as to limit the available space for bioreceptor molecule attachment on the nanobridge surface, with the total number of bioreceptor molecules per nanobridge is limited to less than 20, preferably less than 5, even more preferably less than 3.

Example 29 is directed to the article of Example 27 wherein the gap between the source electrode and drain electrode is further reduced so as to allow only a single bioreceptor molecule attached on the nanobridge.

Example 30 is directed to the article of Example 1 wherein the biosensor structure is combined with a tethered array of memory encoded DNA fragments periodically positioned on a substrate, DNA template and enzyme polymerase array in a microfluidic chamber to enable reading and retrieval of DNA memory.

Example 31 is directed to the article of Example 1 wherein the biosensors are formed on a CMOS based integrated circuit chip that provides the pixel measurement circuits and sensor array readout circuitry to enable an integrated CMOS biosensor chip device.

Example 32 is directed to the article of Example 31 wherein the density of the CMOS biosensors is at least 1,000 sensors/cm², preferably at least 10,000 sensors/cm², more preferably at least 100,000 sensors/cm², even more preferably at least 1 million sensors/cm².

Example 33 is directed to the article of Example 31 wherein the CMOS based biosensors are placed in a microfluidic type detector system, also with support hardware and software control components.

Example 34 is directed to the article of Example 31 wherein the biosensor is CMOS platform nano-bio sensor array based on In₂O₃ nanobridge array onto which biosensor molecules are conjugate attached, with the sputter deposited and patterned In₂O₃ based nanobridges are placed above the source-drain electrodes.

Example 35 is directed to the article of Example 31 wherein the biosensor is CMOS platform nano-bio sensor array based on In₂O₃ nanobridge array onto which biosensor molecules are conjugate attached, with the sputter deposited and patterned In₂O₃ based nanobridges are placed below the source-drain electrodes, with the electrodes confining the length dimension of the In₂O₃ nanobridge opening for restriction of the number of bioreceptor molecules per each sensor.

Example 36 is directed to the article of Example 31 wherein the biosensor comprises: (a) An aptamer molecule is used as the bioreceptor molecule, which has an affinity for histidine-tag on a protein attached onto the In₂O₃ nanobridge surface; (b) With the aptamer functionalized by silane chemistry to detect a nucleic acid-to-protein binding; (c) With the average electric pulse current signal level increasing with increasing target molecule concentration, and increasing frequency of target molecule binding events.

Example 37 is directed to the article of Example 36 wherein the biosensor pulse current signal exhibiting aptamer-protein binding events in the presence of the target receptor binding domain of COVID19 that contained a His-tag at its termini.

Example 38 is directed to the article of Example 31 wherein the biosensor further comprises: (a) A DNA aptamer molecule attached onto the In₂O₃ nanobridge surface is used as the bioreceptor molecule, which has an affinity for hepatitis B surface antigen (HBsAg); (b) With the aptamer functionalized by silane chemistry to detect an antigen-antibody binding; (c) With the average electric pulse current signal level increasing with increasing target molecule concentration, and increasing frequency of target molecule binding events.

Example 39 is directed to the article of Example 34 and Example 35 wherein the In₂0₃ oxide semiconductors nanobridge has controlled oxygen vacancy, with the value of x in a composition of In₂O₃₋ₓ being less than 0.5, preferably less than 0.3, even more preferably less than 0.1 in order to provide higher signal-to-noise ratio during biosensing.

Example 40 is directed to the article of Example 34 and Example 35 wherein the In₂O₃ nanobridge exhibits an electrical resistivity value greater than 50 milliohm-cm, preferably greater than 200 milliohm-cm, more preferably greater than 500 milliohm-cm, even more preferably greater than 1,000 milliohm-cm.

Example 41 is directed to the article of Example 1 wherein the nanobridge has a structure containing a porosity of at least 20%, preferably at least 50%, more preferably at least 70%, with corresponding electrical resistivity increase of at least 20%, preferably at least 50%, more preferably at least 70%.

Example 42 is directed to the article of Example 1 wherein the nanobridge has a structure containing a second phase inclusion of electrically insulating islands with an amount of at least 20%, preferably at least 50%, more preferably at least 70%, with corresponding electrical resistivity increase of at least 20%, preferably at least 50%, more preferably at least 70%.

Example 43 is directed to the article of Example 42 wherein the electrically insulating island material inclusion in the nanobridge is selected from oxide islands of Al₂O₃, SiO₂, MgO, Ta₂O₅, Cr₂O₃, rare earth oxide, nitride islands of AlN, Si₃N₄, rare earth nitride, or fluoride islands of MgF₂,CaF₂, AlF, rare-earth fluoride.

Example 44 is directed to the article of Example 1 wherein the bioreceptor biomolecule is attached onto the semiconductor nanobridge surface only on the anchoring islands placed on the nanobridge surface.

Example 45 is directed to the article of Example 44 wherein the bioreceptor biomolecule is selected from DNA polymerase, peptide, DNA, aptamer.

Example 46 is directed to the article of Example 44 wherein the bioreceptor biomolecule is attached to the anchoring islands by binding moieties and complexes selected from thiol binding, biotin-streptavidin, antigen-antibody, pyrene-maleimide, maleimide-azide, amine-related, silane related complexes, PEG (polyethylene glycol) related complexes,

Example 47 is directed to the article of Example 44 wherein the anchoring islands are made of noble metal material selected from Pt, Pd, Au, Ag, Ru, Rh and their alloys.

Example 48 is directed to the article of Example 44 wherein there is an array of multiple anchoring islands on each nanobridge with associated multiple bioreceptor molecules attached to the anchoring islands.

Example 49 is directed to the article of Example 44 wherein there is only one anchoring island on each nanobridge with a single bioreceptor molecule attached to the anchoring island.

Example 50 is directed to the article of Example 44 wherein the anchoring island dimension is in the range of 5-30 nm, preferably less than 15 nm, more preferably less than 10 nm, even more preferably less than 6 nm.

Example 51 is directed to the article of Example 44 wherein the number of bioreceptor molecule for each anchoring island is 0.5-2, preferably 0.5-1, more preferably 0.7-1.

Example 52 is directed to the article of Example 1 wherein the biosensor comprises a multiplexing structure with multiple regions of sub-divided droplet analyte-containing solutions, each droplet solution in contact with a different nanobridge sensor or an array of sensors for separate but simultaneous assay or sequencing analysis with different patient's samples, or the same patient's samples for different types of analysis.

Example 53 is directed to the article of Example 52 wherein the multiplexing structure comprises a nanopatterned hydrophobic mask coating with an array of hydrophilic openings for easier maintenance of shape and spacing of analyte-containing droplet solutions.

Example 54 is directed to the article of Example 1 wherein the biosensor comprises a multiplexing structure with multiple compartments, with each compartment containing one or more nanobridge sensors in contact with a specified analyte-containing solution, for separate but simultaneous assay or sequencing analysis with different patient's samples, or the same patient's samples for different types of analysis.

Example 55 is directed to a method of preparing an array of biosensor structures for biosensing operations, each biosensor comprising: (a). A source-drain nanoelectrode pair made of a conductive metal, with the electrodes separated by a nanogap; (b). With the conductive metal electrode material selected from Au, Pt, Pd, Ag, Ru, Rh, Ir, Cu, Ni or their alloys; (c). At least one nanobridge in an array made of a metal-nonmetal compound semiconductor connecting each of the two electrode pairs across the nanogap; (d). With the metal-nonmetal compound semiconductor selected from an oxide, carbide, nitride, phosphide or sulfide semiconductor; (e). One or more bioreceptor biomolecules attached onto the semiconductor nanobridge surface to attract and bind with one or more target molecules; (f). With the bioreceptor biomolecule bound to the nanobridge surface by one or more inserted conjugating moiety molecules selected from biotin-streptavidin complex, antigen-antibody complex, pyrene-maleimide complex, connecting moiety comprising azide-related-related click chemistry, amine-related click chemistry, tag-binding related links, or silane-related complexes; (g). With the bioreceptor molecule selected from DNAs, peptides, aptamers, enzyme molecules, DNA Taq polymerase single enzyme molecule, cells, or antibodies; (h). With the sample solution containing target analyte molecules provided, with the target molecule comprising one or more disease-inducing or epidemic-causing virus (coronavirus including COVID-19 virus, SARS or influenza virus, hepatitis, HIV, hepatovirus), or other micro bacteria and organisms, nucleotides in DNA analysis, proteins or DNAs in forensic analysis, enzymes, hormones, albumin, peptides for metabolic and health examinations, sensing of environmental pollutants made of organic or inorganic nature including volatile organic compounds; (i). With the biosensor function based on an attachment or detachment of one or more target molecules onto or from the biosensor, which alters the electrical characteristics of the semiconductor nanobridge to produce electronic pulse signals to enable a detection of the target biomolecule; Wherein the nanobridge array is fabricated by a thin film deposition method selected from sputtering, evaporation, chemical spin coating or atomic layer deposition; Wherein the thin film is nanopatterned into a ribbon geometry nanobridge by a method selected from electron beam lithography, extreme UV lithography, laser lithography, photolithography, or nanoimprint lithography.

Example 56 is directed to the method of Example 55 wherein the semiconductor nanobridge is made of semiconducting oxide, with the nanobridge oxide material selected from; (a). Either n-type semiconducting oxides of ZnO, In₂O₃, SnO₂, (In,Sn)₂O₃ (ITO type), NbO₂, In—Ga—O (IGO), In—Zn—O (IZO), In—Ga—Zn—O (IGZO), In—Al—Zn—O (IAZO), TiO₂, WO₃, and various doped versions of these oxides; or (b). Or p-type semiconductors (such as CuO, Cu₂O, NiO, spinel oxides, CuMO₂ (M=Al, Ga, In, such as CuInO₂, CuAlO₂, CuGaO₂, CuSrO₂, SrCu₂O₂), LaCuOS, LaCuOSe, with various types and concentrations of dopants added as needed.

Example 57 is directed to the method of Example 55 wherein the semiconductor nanobridge is in a thin ribbon configuration with a nanoscale dimension of less than 200 nm in width, preferably less than 50 nm, more preferably less than 20 nm, even more preferably less than 10 nm, and the thickness of the semiconductor nanobridge is less than 50 nm, preferably less than 20 nm, more preferably less than 10 nm, even more preferably less than 2 nm.

Example 58 is directed to the method of Example 55 wherein the biosensor additionally has one or more control gate electrodes present near the nanoelectrode pair as a top-gated, bottom-gated, parallel-gated, perpendicular-gated, or at-angle gated configuration.

Example 59 is directed to the method of Example 55 wherein the biosensor comprises an insulator-gated field effect transistor biosensor comprising an ultrathin, anticorrosion dielectric layer cover on which the sensor biomolecule is attached, with the ultrathin, anticorrosion dielectric layer cover is selected from SiO₂, Si₃N₄, Al₂O₃, ZrO₂, or HfO₂, with the thickness of the ultrathin anticorrosion dielectric layer cover is in the range of 0.2-50 nm, preferably 0.5-10 nm, more preferably 0.5-5 nm.

Example 60 is directed to the method of Example 55 wherein the fabrication of a nanobridge is carried out by deposition and patterning before the two electrodes are deposited so that the gap control between the two electrodes serves as a mask to restrict the number of bioreceptor molecules attached on the nanobridge.

Example 61 is directed to the method of Example 55 wherein the fabrication of a nanobridge is carried out by deposition and patterning on top of the two electrodes after they are fabricated prior to the nanobridge deposition.

Example 62 is directed to the method of Example 55 wherein the number of bioreceptor molecules attached on the nanobridge is controlled by a deposition of a dielectric mask that blocks the attachment of bioreceptor molecules, with one or more selected openings created, which allow the attachment of bioreceptor molecules only in the opening area, so as to restrict the total number of bioreceptor molecules attached on its surface.

Example 63 is directed to the method of Example 62 wherein the dielectric mask material is processed to contain a selected number of openings as follows; A thin film dielectric material is deposited by sputtering, evaporation, atomic layer deposition, chemical vapor deposition, electrochemical deposition, spin coating of a precursor liquid into a thin film followed by curing, dip-coating and curing, spray-coating and curing, with the dielectric mask film is nanopatterned by a method selected from e-beam lithography, EUV-lithography, photolithography, laser lithography, and nanoimprint lithography.

Example 64 is directed to the method of Example 62 wherein the dielectric mask material is selected from a polymeric material, a ceramic material or a composite material nanopatterned into nanoscale opening of circular, square, rectangular or irregular shape on each nanobridge, or a slit-type opening spanning multiple nanobridges, for selective bioreceptor molecule attachment.

Example 65 is directed to the method of Example 62 wherein the dielectric mask material is selected from of polymethylmethacrylate (PMMA), SU-8, polytetrafluoroethylene (PTFE), polyethylene glycol (PEG), polydimethylsiloxane (PDMS), related elastomers, polystyrene (PS), and polyurethane (PU), or SiO₂, Al₂O₃, MgO, TiO₂, ZrO₂, Ta₂O₅ layer, with a thickness of less than 200 nm, preferably less than 100 nm, more preferably less than 20 nm, even more preferably less than 5 nm.

Example 66 is directed to the method of Example 62 wherein the opening in the mask for bioreceptor molecule attachment has an average equivalent diameter of less than 1 um, preferably less than 500 nm, more preferably less than 100 nm, even more preferably less than 50 nm.

Example 67 is directed to the method of Example 64 wherein the slit-type opening for bioreceptor molecule attachment has a width of opening less than 1 um, preferably less than 500 nm, more preferably less than 100 nm, even more preferably less than 50 nm.

Example 68 is directed to the method of Example 62 wherein the pre-selected open locations on the nanobridge selectively allows different types of bioreceptor molecules, with the surface of each opening area pre-treated or pre-coated with one or more selected functionalities to enhance attachment of specific probe molecule types on the specific desired open hole locations.

Example 69 is directed to the methods of Example 55 to Example 68 wherein the nanobridge material is In₂O₃ based thin film nanobridge.

Example 70 is directed to the method of Example 69 wherein the electrical resistivity value of the In₂O₃ nanobridge material is controlled to be greater than 50 milliOhm-cm, preferably greater than 200 milliOhm-cm, more preferably greater than 500 milliOhm-cm, even more preferably greater than 1,000 milliOhm-cm, by using one of the processing methods listed below; (a). Oxygen partial pressure in the In₂O₃ thin film deposition chamber; (b). By changing the mix ratio of oxygen gas and argon gas flow rate into thin film deposition chamber; (c). Adjusting the post-deposition annealing temperature and time, preferably in an oxygen-rich environment; (d). Altering the bias voltage applied during sputter deposition.

Example 71 is directed to the method of Example 55 wherein a thickness reduction and a width reduction of the semiconductor nanobridge is achieved by a chemical etch erosion, electro-chemical etch erosion or plasma etch erosion of the exposed region of the nanobridge top surface and side edges not covered by the source-drain electrode pair above the nanobridge, or not covered by an etch barrier mask layer intentionally added and nanopatterned over the nanobridge.

Example 72 is directed to the method of Example 71 wherein the resultant reduction of cross-sectional area of the nanobridge is at least by 10%, preferably by at least 30%, more preferably by at least 50%.

Example 73 is directed to the method of Example 55 wherein a thickness reduction and a width reduction of the semiconductor nanobridge is achieved by use of erodible mask line array following the steps of: (a). Deposition of erodible mask lines by thin film deposition or spin-coating deposition of a layer selected from a nano patternable resist layer or metallic layer; (b). Nanopatterning of the erodible layer into line mask array using e-beam lithography, photolithography, extreme UV (EUV) lithography, nanoimprint lithography, or laser nanofabrication lithography; (c). Apply an etching erosion on the erodible mask line material itself in order to reduce the width of the mask line, using one of the processes listed below, (i) RIE (reactive ion etch), (ii) chemical or electrochemical etch including acid etching, (iii) solvent etching, (iv) repeated (oxidation+etching) for gradual width reduction, (v) edge preferential oxidation, nitriding, sulfurization followed by edge preferential etching, (vi) atomic layer etching or intentional e-beam damage of edge for more preferential etching away for width reduction; (d). Perform etching of the nanobridge material by lithography processing using the width-reduced mask line array, so that a narrower nanobridge is obtained.

Example 74 is directed to the method of Example 73 wherein the resultant reduction of cross-sectional area of the nanobridge by using the erodible mask line is at least by 10%, preferably by at least 30%, more preferably by at least 50%.

Example 75 is directed to the method of Example 55 wherein the nanobridge is oblique incident sputtered so as to introduce a porosity of at least 20%, preferably at least 50%, more preferably at least 70%, with corresponding electrical resistivity increase of at least 20%, preferably at least 50%, more preferably at least 70% within the nanobridge within the nanobridge material.

Example 76 is directed to the method of Example 55 wherein the nanobridge is made porous by incorporating dissolvable metal, ceramic or carbon-containing material which is selectively etched away or burned away to introduce a porosity of at least 20%, preferably at least 50%, more preferably at least 70%, with corresponding electrical resistivity increase of at least 20%, preferably at least 50%, more preferably at least 70% within the nanobridge within the nanobridge material.

Example 77 is directed to the method of Example 55 wherein the nanobridge is made to contain insulating islands by co-deposition selected from co-sputtering, co-evaporation, or co-electrodeposition so that the amount of insulating islands is at least 20%, preferably at least 50%, more preferably at least 70%.

Example 78 is directed to the method of Example 55 wherein the nanobridge contains surface anchoring islands for preferential attachment of bioreceptor senor molecules with the anchoring islands are prepared by depositing noble metal film followed by a lithographic process, selected from electron beam lithography, laser lithography, extreme UV lithography, or nanoimprint lithography.

Example 79 is directed to the method of Example 55 wherein a multiplexing biosensing analysis is performed by placing sub-divided droplet analyte-containing solutions at multiple regions, with each region containing a section of nanobridge sensor array to allow separate but simultaneous assay or sequencing analysis with different patient's samples, or the same patient's samples for different types of analysis.

Example 80 is directed to the method of Example 55 wherein a multiplexing biosensing analysis is performed by placing sub-divided droplet analyte-containing solutions in each of the multiple compartments, with each compartment containing a section of nanobridge sensor array to allow separate but simultaneous assay or sequencing analysis with different patient's samples, or the same patient's samples for different types of analysis.

Examples on Nanobridge Transfer Deposit methods-Example 81 is directed to a method of preparing an array of biosensor structures, each biosensor comprising: (a). A nanoelectrode pair made of a conductive metal, with the electrodes separated by a nanogap; (b). With the conductive metal electrode material selected from Au, Pt, Pd, Ag, Ru, Rh, Ir, Cu, Ni or their alloys; (c). At least one nanobridge in an array made of a metal-nonmetal compound semiconductor connecting each of the two electrode pairs across the nanogap; (d). With the metal-nonmetal compound semiconductor selected from an oxide, carbide, nitride, phosphide or sulfide semiconductor; (e). One or more bioreceptor biomolecules attached onto the semiconductor nanobridge surface to attract and bind with one or more target molecules; (f). With the bioreceptor biomolecule bound to the nanobridge surface by one or more inserted conjugating moiety molecules selected from biotin-streptavidin complex, antigen-antibody complex, pyrene-maleimide complex, connecting moiety comprising azide-related-related click chemistry, amine-related click chemistry, tag-binding related links, or silane-related complexes; (g). With the bioreceptor molecule selected from DNAs, peptides, aptamers, enzyme molecules, DNA Taq polymerase single enzyme molecule, cells, or antibodies; (h). With the sample solution containing target analyte molecules provided, with the target molecule comprising one or more disease-inducing or epidemic-causing virus (coronavirus including COVID-19 virus, SARS or influenza virus, hepatitis, HIV, hepatovirus), or other micro bacteria and organisms, nucleotides in DNA analysis, proteins or DNAs in forensic analysis, enzymes, hormones, albumin, peptides for metabolic and health examinations, sensing of environmental pollutants made of organic or inorganic nature including volatile organic compounds; (i). With the biosensor function based on an attachment or detachment of one or more target molecules onto the biosensor, which alters the electrical characteristics of the semiconductor nanobridge to produce electronic pulse signals to enable a detection of the target biomolecules; Wherein the semiconductor nanobridge array is fabricated on a separate, temporary substrate which is not the final device substrate using a thin film deposition method selected from sputtering, evaporation, chemical spin coating or atomic layer deposition; Wherein the thin film is nanopatterned into an array of nanoribbon nanobridges by a method selected from e-beam lithography, EUV-lithography, laser lithography, photolithography, or nanoimprint lithography; Wherein the array of fabricated nanoribbon nanobridges is optionally subjected to annealing heat treatment in a gas environment containing air, oxygen, nitrogen, nitrogen-containing gas, hydrogen, sulfur, phosphor, carbon-containing gas, and their mixtures, or vacuum environment to optimize the nanobridge semiconductor physical and electrical properties; Wherein the array of fabricated nanoribbon nanobridges is optionally subjected to doping treatment, ion implantation treatment, electron-beam or ion beam treatment, acid or plasma ion etch treatment, or other chemical solution surface modifying treatment; Then the fabricated nanobridge array is released from the temporary substrate, optionally utilizing a sacrificial, dissolvable layer underneath, picked up by elastomeric stamp or adhesive stamp, and transfer deposited onto the final device substrate comprising the source-drain electrode array, and released.

Example 82 is directed to the method of Example 81 wherein the semiconductor nanoribbon array is fabricated on the separate substrate in a pre-aligned parallel manner, and transfer deposited onto the parallel electrode pair surface, with the spacing between adjacent nanoribbons selected to be less than the width of the electrodes so that there is always at least one nanoribbon falling on electrode pair surface during the transfer deposit.

Example 83 is directed to the method of Example 81 wherein the elastomeric or adhesive stamp contacts the top of the nanoelectrode pairs during compressive transfer deposit, with the height of the nanoelectrode pair made to be sufficient to prevent the stamp to contact the empty space between the adjacent electrode pairs thus preventing a transfer deposit of the nanoribbon in the empty space, which results in the transfer deposit of the nanoribbons only on the protruding electrode surface.

Example 84 is directed to the method of Example 81 wherein the nanoelectrodes is made to vertically protrude by at least 10 nm, preferably at least 20 nm, more preferably at least 50 nm, and even more preferably at least 100 nm.

Example 85 is directed to the method of Example 81 wherein the semiconductor nanobridge is in a thin ribbon configuration with a nanoscale dimension of less than 200 nm in width, preferably less than 50 nm, more preferably less than 20 nm, even more preferably less than 10 nm.

Example 86 is directed to the method of Example 81 wherein the thickness of the semiconductor nanobridge is less than 50 nm, preferably less than 20 nm, more preferably less than 10 nm, even more preferably less than 2 nm.

Example 87 is directed to the method of Example 81 wherein the number of bioreceptor molecules attached on the transfer deposited nanobridge is controlled by a deposition of a dielectric mask that blocks the attachment of bioreceptor molecules, with one or more openings created, which allow the attachment of bioreceptor molecules only in the opening area, so as to restrict the total number of bioreceptor molecules attached on its surface.

Example 88 is directed to the method of Example 87 wherein the dielectric mask material is processed to contain a selected number of openings as follows; A thin film dielectric material is deposited by sputtering, evaporation, atomic layer deposition, chemical vapor deposition, electrochemical deposition, spin coating of a precursor liquid into a thin film followed by curing, dip-coating and curing, spray-coating and curing, With the dielectric mask film is nanopatterned by a method selected from e-beam lithography, EUV-lithography, photolithography, laser lithography, and nanoimprint lithography.

Example 89 is directed to the method of Example 87 wherein the dielectric mask material is selected from a polymeric material, a ceramic material or a composite material nanopatterned into nanoscale opening of circular, square, rectangular or irregular shape on each nanobridge, or a slit-type opening spanning multiple nanobridges, for selective bioreceptor molecule attachment.

Example 90 is directed to the method of Example 87 wherein the dielectric mask material is selected from of polymethylmethacrylate (PMMA), SU-8, polytetrafluoroethylene (PTFE), polyethylene glycol (PEG), polydimethylsiloxane (PDMS), related elastomers, polystyrene (PS), and polyurethane (PU), or SiO₂, Al₂O₃, MgO, TiO₂, ZrO₂, Ta₂O₅ layer, with a thickness of less than 200 nm, preferably less than 100 nm, more preferably less than 20 nm, even more preferably less than 5 nm.

Example 81 is directed to the method of Example 87 wherein the opening in the mask for bioreceptor molecule attachment has an average equivalent diameter of less than 1 um, preferably less than 500 nm, more preferably less than 100 nm, even more preferably less than 50 nm.

Example 92 is directed to the method of Example 89 wherein the slit-type opening for bioreceptor molecule attachment has a width of opening less than 1 um, preferably less than 500 nm, more preferably less than 100 nm, even more preferably less than 50 nm.

Example 93 is directed to the method of Example 87 wherein the open locations on the nanobridge through the dielectric mask selectively allows different types of bioreceptor molecules, with the surface of each opening area pre-treated or pre-coated with one or more selected functionalities to enhance attachment of specific probe molecule types on the specific desired open hole locations.

Example 94 is directed to the methods of Example 81 to Example 93 wherein the nanobridge material is In₂O₃ based semiconductors thin film nanobridge having a controlled oxygen vacancy, with the value of x in a composition of In₂O₃₋ₓ being less than 0.5, preferably less than 0.3, even more preferably less than 0.1 in order to provide higher signal-to-noise ratio during biosensing.

Example 95 is directed to the method of Example 94 wherein the electrical resistivity value of the In₂O₃ nanobridge material is controlled to be greater than 50 milliohm-cm, preferably greater than 200 milliohm-cm, more preferably greater than 500 milliohm-cm, even more preferably greater than 1,000 milliohm-cm, by using one of the processing methods listed below; (a). Oxygen partial pressure in the In₂O₃ thin film deposition chamber; (b). By changing the mix ratio of oxygen gas and argon gas flow rate into thin film deposition chamber; (c). Adjusting the post-deposition annealing temperature and time, preferably in an oxygen-rich environment; (d). Altering the bias voltage applied during sputter deposition.

Example 96 is directed to the article of Example 1 wherein the biosensor structure is combined with a tethered array of memory encoded DNA fragments periodically positioned on a substrate, DNA template and enzyme polymerase array in a microfluidic chamber to enable reading and retrieval of DNA memory.

Example 97 is directed to the article of Example 1 wherein the biosensors are formed on a CMOS based integrated circuit chip that provides the pixel measurement circuits and sensor array readout circuitry to enable an integrated CMOS biosensor chip device to detect pulse current signals for target molecule binding.

Example 98 is directed to the article of Example 97 wherein the density of the CMOS biosensors is at least 1,000 sensors/cm² , preferably at least 10,000 sensors/cm² , more preferably at least 100,000 sensors/cm² , even more preferably at least 1 million sensors/cm².

Example 99 is directed to the article of Example 97 wherein the average spacing between adjacent CMOS based biosensors is less than 100 micrometers, preferably less than 10 micrometers, more preferably less than 5 micrometers, even more preferably less than 1 um.

Example 100 is directed to the article of Example 97 wherein the CMOS based biosensors are placed in a microfluidic type detector system, also with support hardware and software control components.

Example 101 is directed to the article of Example 97 wherein the biosensor is CMOS platform nano-bio sensor array based on In₂O₃ nanobridge array onto which biosensor molecules are conjugate attached, with the sputter deposited and patterned In₂O₃ nanobridges placed above the source-drain electrodes.

Example 102 is directed to the article of Example 97 wherein the biosensor is CMOS platform nano-bio sensor array based on In₂O₃ nanobridge array onto which biosensor molecules are conjugate attached, with the sputter deposited and patterned In₂O₃ nanobridges are placed below the source-drain electrodes, with the electrodes confining the length dimension of the In₂O₃ nanobridge opening for restriction of the number of bioreceptor molecules per each sensor.

Example 103 is directed to the article of Example 97 wherein the biosensor comprises: (a) An aptamer molecule is used as the bioreceptor molecule, which has an affinity for histidine-tag on a protein attached onto the In₂O₃ nanobridge surface; (b) With the aptamer functionalized by silane chemistry to detect a nucleic acid-to-protein binding; (c) With the average electric pulse current signal level increasing with increasing target molecule concentration, and increasing frequency of target molecule binding events.

Example 104 is directed to the article of Example 97 wherein the biosensor pulse current signal exhibiting aptamer-protein binding events in the presence of the target receptor binding domain of COVID19 that contained a His-tag at its termini.

Example 105 is directed to the article of Example 97 wherein the biosensor comprises; (a) A DNA aptamer molecule attached onto the In₂O₃ nanobridge surface is used as the bioreceptor molecule, which has an affinity for hepatitis B surface antigen (HBsAg); (b) With the aptamer functionalized by silane chemistry to detect an antigen-antibody binding; (c) With the average electric pulse current signal level increasing with increasing target molecule concentration, and increasing frequency of target molecule binding events.

Example 106 is directed to the article of Examples 101 and 102 wherein the In₂O₃nanobridge exhibits an electrical resistivity value greater than 50 milliOhm-cm, preferably greater than 200 milliOhm-cm, more preferably greater than 500 milliOhm-cm, even more preferably greater than 1,000 milliOhm-cm.

Example 107 is directed to the article of Example 1 wherein the biosensor device is used for detection of disease-causing analyte target biomolecules.

Example 108 is directed to the article of Example 1 wherein the disease-causing target biomolecules are virus, bacteria or micro organisms.

Example 109 is directed to the biosensor device of Example 107 wherein the disease-causing analyte target biomolecules are one of the following; epidemic-causing coronavirus including COVID-19 virus, SARS or influenza virus, hepatitis, HIV, hepatovirus.

Example 110 is directed to the article of Example 1 wherein the biosensor device is used for DNA analysis for genome sequencing.

Example 111 is directed to the article of Example 1 wherein the biosensor device is used for forensic analysis of DNAs and organic molecules.

Example 112 is directed to the article of Example 1 wherein the biosensor device is used for analysis of human or animal enzymes, hormone levels, albumin, peptides, electrolyte levels, pH levels, metabolic status, for health examinations.

Example 113 is directed to the article of Example 1 wherein the biosensor device is used for detection of environmental pollutants including volatile organic compounds.

Example 114 is directed to the article of Example 1 wherein the biosensor device is used for detection of air quality for health maintenance.

Example 115 is directed to the article of Example 1 wherein the biosensor device is used for detection of odor releases for entertainment, food spoil and decay monitoring, perfume discrimination and environmental pollutants including volatile organic compounds.

Example 116 is directed to the article of Example 1 wherein the biosensor device is used for detection of release quality of odors and perfumes for meta verse experience, virtual reality and augmented reality experience, gaming experience, on0line purchase of odor, perfume, flavor product evaluations and purchases.

Example 117 is directed to the article of Example 1 wherein the biosensor device is used for analysis of blood samples.

Example 118 is directed to the article of Example 1 wherein the biosensor device is used for analysis of human sweat samples.

Example 119 is directed to the article of Example 1 wherein the biosensor device is used for analysis of saliva samples.

Example 120 is directed to the article of Example 1 wherein the biosensor device is used for analysis of nasal mucus samples.

Example 121 is directed to the article of Example 1 wherein the biosensor device is used for analysis of urine samples.

Example 122 is directed to the article of Example 1 wherein the biosensor device is wearable.

Example 123 is directed to the article of Example 1 wherein the biosensor device is on a portable platform to be carryable.

It is noted that the disclosed embodiments are exemplary and are not intended to limit the scope of the disclosed principles or the embodiments themselves. Any equivalent modification or variation according to the spirit of any of the embodiments disclosed herein is to be also included within the scope of any of the embodiments disclosed herein. For example, instead of rigid Si based or SiO₂ coated substrates, flexible plastic substrates such as based on PET (polyethylene terephthalate), PMMA (polymethyl methacrylate) or PEN (polyethylene naphthalate) could be utilized to place metal-nonmetal semiconductor nanobridges such as In₂O₃ In₂O₃ nanobridges and bioreceptor sensor molecules in order to produce wearable biosensors.

What is claimed is:

1. An array of biosensor, at least one biosensor in the array comprising:
an electrode pair having a source electrode and a drain electrode separated by a nanogap;
a nanobridge including a metal-nonmetal compound semiconductor connecting the two electrodes across the nanogap;
one or more bioreceptors attached to the nanobridge;
an analyte solution comprising water, salts and one or more target molecules;
a reaction chamber for housing the array and for receiving the analyte solution;
a circuitry configured to detect and measure a change in electrical characteristics of the analyte when a respective one of the target molecules attaches or detaches from a respective one of the bioreceptors;
wherein the biosensor detects a change in the electrical characteristic of the nanobridge during the event.

2. The array of claim 1, further comprising a plurality of bioreceptors wherein the placement of the bioreceptors on the nanobridge is predefined by a mask layer having a plurality of openings to couple each bioreceptor to the nanobridge through one of the respective openings.

3. The array of claim 1, wherein the nanobridge comprises semiconducting oxide, with the nanobridge oxide material selected from the group consisting of n-type semiconducting oxides (such as ZnO, In₂O₃, SnO₂, (In,Sn)₂O₃ (ITO type), NbO₂, In—Ga—O (IGO), In—Zn—O (IZO), In—Ga—Zn—O (IGZO), In—Al—Zn—O (IAZO), TiO₂, WO₃ and various doped versions of these oxides.

4. The array of claim 1, wherein the nanobridge comprises of semiconducting oxide, with the nanobridge oxide material selected from the group consisting of p-type semiconductors (such as CuO, Cu₂O, NiO, spinel oxides, CuMO₂ (M=Al, Ga, In, such as CuInO₂, CuAlO₂, CuGaO₂, CuSrO₂, SrCu₂O₂), LaCuOS, LaCuOSe and dopants.

5. The array of claim 1, wherein the source or the drain electrodes comprise a conductive metallic material selected from the group consisting of Au, Pt, Pd, Ag, Ru, Rh, Ir, Cu, Ni and alloys thereof.

6. The array of claim 1, wherein the said bioreceptor biomolecule is coupled to the nanobridge by one or more conjugating moiety molecules selected from the group consisting of thiol binding, biotin-streptavidin complex, antigen-antibody complex, pyrene-maleimide complex, connecting moiety comprising maleimide-azide, azide-related click chemistry, amine-related click chemistry, tag-binding related links, silane-related, and PEG (polyethylene glycol) related complexes.

7. The array of claim 1, wherein the said bioreceptor biomolecule is selected from the group consisting of DNAs, peptides, aptamers, enzyme molecules, DNA Taq polymerase single enzyme molecule, cells and antibodies.

8. The array of claim 1, wherein the target molecule comprises a disease-inducing virus, micro bacteria, organisms, nucleotides, proteins, DNA, enzymes, hormones, albumin, peptides, pollutants or volatile organic compounds.

9. The array of claim 8, wherein the target molecule is extracted from a mammalian biofluid, blood, sweat, saliva, mucus, endocrine glands or urine.

10. The array of claim 8, wherein the target molecule is selected from the group consisting of DNAs, proteins, bacteria and virus.

11. The array of claim 1, wherein the nanobridge defines a ribbon configuration with a width of less than about 200, 50, 20 or 10 nm.

12. The array of claim 1, wherein the nanobridge defines a ribbon configuration with a thickness of less than about 50, 20, 10 or 2 nm.

13. The array of claim 1, further comprising a gate electrode in communication with the electrode pair, wherein the gate electrode is positioned as a top-gate, bottom-gate, parallel-gate, perpendicular-gate or at-angle gate relative to the electrode pair.

14. The array of claim 1, wherein bioreceptor molecule defines an aptamer with an affinity for histidine-tag on a protein attached onto the nanobridge surface, the aptamer functionalized by silane chemistry to detect a nucleic acid-to-protein binding, and wherein the average electric pulse current signal level increases with an increase in the target molecule concentration in the analyte or an increase in the frequency of the event.

15. The array of claim 1, wherein the nanobridge has a structure containing a porosity of at least about 20%, 50% or 70% and wherein the nanobridge has a structure containing a second phase inclusion of electrically insulating islands with an amount of a least 20%, 50%, 70%.

16. The array of claim 1, wherein the nanobridge has a size and location defined mask structure with openings to allow tethering attachment of bioreceptor numbers selected from a single bioreceptor molecule, or 2, 3, 4, 5, 10, 20 or more bioreceptor molecules in an array only at specific pre-defined locations on the nanobridge surface.

17. A method for preparing an array of biosensor structures for biosensing operations, each biosensor comprising:
a source-drain nanoelectrode pair made of a conductive metal, with the electrodes separated by a nanogap, wherein the conductive metal material is selected from the group consisting of Au, Pt, Pd, Ag, Ru, Rh, Ir, Cu, Ni and alloys thereof;
a nanobridge disposed in the nanogap and connecting the source and the drain electrodes, the nanobridge comprising a metal-nonmetal compound semiconductor connecting each of the two electrode pairs across the nanogap, wherein the metal-nonmetal compound semiconductor selected from an oxide, carbide, nitride, phosphide or sulfide semiconductor;
a bioreceptor biomolecules coupled to the semiconductor nanobridge to attract and bindingly engage with a target molecule, with the number and location of the bioreceptor molecules controlled by a mask layer comprising pre-defined openings to allow bioreceptor tethering attachment;
wherein the bioreceptor binds to the nanobridge by one or more inserted conjugating moiety molecules selected from the group consisting of biotin-streptavidin complex, antigen-antibody complex, pyrene-maleimide complex, connecting moiety comprising azide-related -related click chemistry, amine-related click chemistry, tag-binding related links, and silane-related complexes.

18. The method of claim 17, wherein the bioreceptor molecule is selected from the group consisting of DNAs, peptides, aptamers, enzyme molecules, DNA Taq polymerase single enzyme molecule, cells and antibodies.

19. The method of claim 17, further comprising an analyte, the analyte including the target molecule and wherein the target molecule further comprises one or more a virus, a bacterium, and organism, a nucleotide, a protein, a pollutant, an organic or an inorganic compound.
